SEQUENCE LISTING (290 sequences)

SEQ ID NO: 1 - RNA, 15 nt, synthetic construct
  source 1..15: mol_type = other RNA
aaaaaaaaaaaaaaa

SEQ ID NO: 2 - RNA, 22 nt, synthetic construct
  source 1..22: mol_type = other RNA
gctactgaaaaaaaaaaaaaaa

SEQ ID NO: 3 - RNA, 22 nt, synthetic construct
  source 1..22: mol_type = other RNA
aaaaaaaaaaaaaaagtcatcg

SEQ ID NO: 4 - RNA, 26 nt, synthetic construct
  source 1..26: mol_type = other RNA
aaaaaaagccgttgacggcaaaaaaa

SEQ ID NO: 5 - DNA, 15 bp, synthetic construct
  source 1..15: mol_type = other DNA
aaaaaaaaaaaaaaa

SEQ ID NO: 6 - DNA, 30 bp, synthetic construct
  source 1..30: mol_type = other DNA
aaaaaaaaaaaaaaaaaaaaaaaaaaaaaa

SEQ ID NO: 7 - DNA, 54 bp, synthetic construct
  source 1..54: mol_type = other DNA
aaaaaaaaaaaaaaaaaaaaaaaaaaaaaaccccattctacttgagagagcgac

SEQ ID NO: 8 - DNA, 38 bp, synthetic construct
  source 1..38: mol_type = other DNA
aaaaaaaaaaaaaaaaaaaaaaaaaaaaaacccccccc

SEQ ID NO: 9 - DNA, 34 bp, synthetic construct
  source 1..34: mol_type = other DNA
aaaaaaaaaaaaaaaaaaaaaaaaaaaaaacccc

SEQ ID NO: 10 - DNA, 54 bp, synthetic construct
  source 1..54: mol_type = other DNA
aaaaaaaaaaaaaaaaaaaaaaaaaaaaaaccccgtcgctctctcaagtagaat

SEQ ID NO: 11 - DNA, 47 bp, synthetic construct
  source 1..47: mol_type = other DNA
gatgaggtacccagaagccgaactgggccagacaacccggcgctaac

SEQ ID NO: 12 - DNA, 76 bp, synthetic construct
  source 1..76: mol_type = other DNA
aaaaaaaaaaaaaaagatgaggtacccagaagccgaactgggccagacaacccggcgctaacaaaaaaaaaaaaaa

SEQ ID NO: 13 - DNA, 18 bp, synthetic construct
  source 1..18: mol_type = other DNA
atatccttgtcgtatccc

SEQ ID NO: 14 - DNA, 19 bp, synthetic construct
  source 1..19: mol_type = other DNA
aataattgaattcttcnaa

SEQ ID NO: 15 - DNA, 46 bp, synthetic construct
  source 1..46: mol_type = other DNA
aaaaaaaaaaaaaaaaaaaaaaaaaaaaaattacaagcaggttcct

SEQ ID NO: 16 - DNA, 49 bp, synthetic construct
  source 1..49: mol_type = other DNA
aaaaaaaaaaaaaaaaaaaaaaaaaaaaaattacttgaagaattcaatt

SEQ ID NO: 17 - RNA, 60 nt, synthetic construct
  source 1..60: mol_type = other RNA
aaaaaaaaaaaaaaatcaccacgtaaacatgggtcttagttgactgtaccatctgcaacg

SEQ ID NO: 18 - RNA, 45 nt, synthetic construct
  source 1..45: mol_type = other RNA
aaaaaaaaaaaaaaatcaccacgtaaacatgggtcttagttgact

SEQ ID NO: 19 - RNA, 39 nt, synthetic construct
  source 1..39: mol_type = other RNA
aaaaaaaaaaaaaaatcaccacgtaaacatgggtcttag

SEQ ID NO: 20 - RNA, 30 nt, synthetic construct
  source 1..30: mol_type = other RNA
aaaaaaaaaaaaaaatcaccacgtaaacat

SEQ ID NO: 21 - DNA, 30 bp, synthetic construct
  source 1..30: mol_type = other DNA
aaaaaaaaaaaaaaatcaccacgtaaacat

SEQ ID NO: 22 - DNA, 60 bp, synthetic construct
  source 1..60: mol_type = other DNA
aaaaaaaaaaaaaaatcaccacgtaaacatgggtcttagttgactgtaccatctgcaacg

SEQ ID NO: 23 - DNA, 45 bp, synthetic construct
  source 1..45: mol_type = other DNA
aaaaaaaaaaaaaaatcaccacgtaaacatgggtcttagttgact

SEQ ID NO: 24 - DNA, 39 bp, synthetic construct
  source 1..39: mol_type = other DNA
aaaaaaaaaaaaaaatcaccacgtaaacatgggtcttag

SEQ ID NO: 25 - DNA, 54 bp, synthetic construct; identical to SEQ ID NO: 7
  source 1..54: mol_type = other DNA

SEQ ID NO: 26 - DNA, 34 bp, synthetic construct; identical to SEQ ID NO: 9
  source 1..34: mol_type = other DNA

SEQ ID NO: 27 - DNA, 54 bp, synthetic construct; identical to SEQ ID NO: 10
  source 1..54: mol_type = other DNA

SEQ ID NO: 28 - DNA, 50 bp, synthetic construct
  source 1..50: mol_type = other DNA
aaaaaaaaaaaaaaaaaaaaaaaaaaaaaaaaaaaaaaaaaaaaaaaaaa

SEQ ID NO: 29 - DNA, 34 bp, synthetic construct
  source 1..34: mol_type = other DNA
aaaaaaaaaaaaaaaaaaaaaaaaaaaaaagggg

SEQ ID NO: 30 - DNA, 38 bp, synthetic construct
  source 1..38: mol_type = other DNA
aaaaaaaaaaaaaaaaaaaaaaaaaaaaaagggggggg

SEQ ID NO: 31 - DNA, 47 bp, synthetic construct
  source 1..47: mol_type = other DNA
aaaaaaaaaaaaaaaaaaaaaaaaaaaaaactagaagtgtccagtta

SEQ ID NO: 32 - DNA, 47 bp, synthetic construct
  source 1..47: mol_type = other DNA
aaaaaaaaaaaaaaaaaaaaaaaaaaaaaattactaactggacactt

SEQ ID NO: 33 - DNA, 54 bp, synthetic construct; identical to SEQ ID NO: 7
  source 1..54: mol_type = other DNA

SEQ ID NO: 34 - DNA, 54 bp, synthetic construct; identical to SEQ ID NO: 10
  source 1..54: mol_type = other DNA

SEQ ID NO: 35 - DNA, 113 bp, synthetic construct
  source 1..113: mol_type = other DNA
ctcagcagcgaaaaaccgctttacaacattcgaggcacgttgtacgtccacacttggaacctcatcgcacatccgcctgccacgctcttagcataggacggcggcgttaaata

SEQ ID NO: 36 - DNA, 84 bp, synthetic construct
  source 1..84: mol_type = other DNA
cggtgcatttcgacggtacttcgtacaacgtgcctcgaatgtagagcgtggcaggcggatgtgaagcagttgcagcgtactcgt

SEQ ID NO: 37 - DNA, 63 bp, synthetic construct
  source 1..63: mol_type = other DNA
tcggcagactaatacacctgtcgatgaggttccaagtgtggatagctaggtaacggattgagc

SEQ ID NO: 38 - DNA, 100 bp, synthetic construct
  source 1..100: mol_type = other DNA
aaaaaaaaaaaaaaaaaaaaaaaaaaaaaacgagtacgctgcaactgctaccaggtgtattagtctgccgaaaaaaaaaaaaaaaaaaaaaaaaaaaaaa

SEQ ID NO: 39 - DNA, 100 bp, synthetic construct
  source 1..100: mol_type = other DNA
aaaaaaaaaaaaaaaaaaaaaaaaaaaaaactcaatccgttacctagctccagtaccgtcgaaatgcaccaaaaaaaaaaaaaaaaaaaaaaaaaaaaaa

SEQ ID NO: 40 - DNA, 100 bp, synthetic construct
  source 1..100: mol_type = other DNA
aaaaaaaaaaaaaaaaaaaaaaaaaaaaaaaacgccgccgtcctatgctttgtaaagcggtttttcgctgaaaaaaaaaaaaaaaaaaaaaaaaaaaaaa

SEQ ID NO: 41 - DNA, 40 bp, synthetic construct
  source 1..40: mol_type = other DNA
cgagtacgctgcaactgctaccaggtgtattagtctgccg

SEQ ID NO: 42 - DNA, 40 bp, synthetic construct
  source 1..40: mol_type = other DNA
ctcaatccgttacctagctccagtaccgtcgaaatgcacc

SEQ ID NO: 43 - DNA, 38 bp, synthetic construct
  source 1..38: mol_type = other DNA
cgccgccgtcctatgctttgtaaagcggtttttcgctg

SEQ ID NO: 44 - DNA, 32 bp, synthetic construct
  source 1..32: mol_type = other DNA
caagcccaataggaacccatgtacaaacagtt

SEQ ID NO: 45 - DNA, 32 bp, synthetic construct
  source 1..32: mol_type = other DNA
aatgccccgtaacagtgcccgtatctccctca

SEQ ID NO: 46 - DNA, 32 bp, synthetic construct
  source 1..32: mol_type = other DNA
tgccttgactgcctatttcggaacagggatag

SEQ ID NO: 47 - DNA, 32 bp, synthetic construct
  source 1..32: mol_type = other DNA
gagccgccccaccaccggaaccgcgacggaaa

SEQ ID NO: 48 - DNA, 32 bp, synthetic construct
  source 1..32: mol_type = other DNA
aaccagagaccctcagaaccgccaggggtcag

SEQ ID NO: 49 - DNA, 32 bp, synthetic construct
  source 1..32: mol_type = other DNA
ttattcatagggaaggtaaatattcattcagt

SEQ ID NO: 50 - DNA, 32 bp, synthetic construct
  source 1..32: mol_type = other DNA
cataacccgaggcatagtaagagctttttaag

SEQ ID NO: 51 - DNA, 32 bp, synthetic construct
  source 1..32: mol_type = other DNA
attgagggtaaaggtgaattatcaatcaccgg

SEQ ID NO: 52 - DNA, 32 bp, synthetic construct
  source 1..32: mol_type = other DNA
aaaagtaatatcttaccgaagcccttccagag

SEQ ID NO: 53 - DNA, 32 bp, synthetic construct
  source 1..32: mol_type = other DNA
gcaatagcgcagatagccgaacaattcaaccg

SEQ ID NO: 54 - DNA, 32 bp, synthetic construct
  source 1..32: mol_type = other DNA
cctaatttacgctaacgagcgtctaatcaata

SEQ ID NO: 55 - DNA, 32 bp, synthetic construct
  source 1..32: mol_type = other DNA
tcttaccagccagttacaaaataaatgaaata

SEQ ID NO: 56 - DNA, 32 bp, synthetic construct
  source 1..32: mol_type = other DNA
atcggctgcgagcatgtagaaacctatcatat

SEQ ID NO: 57 - DNA, 32 bp, synthetic construct
  source 1..32: mol_type = other DNA
ctaatttatctttccttatcattcatcctgaa

SEQ ID NO: 58 - DNA, 32 bp, synthetic construct
  source 1..32: mol_type = other DNA
gcgttatagaaaaagcctgtttagaaggccgg

SEQ ID NO: 59 - DNA, 32 bp, synthetic construct
  source 1..32: mol_type = other DNA
gctcattttcgcattaaatttttgagcttaga

SEQ ID NO: 60 - DNA, 32 bp, synthetic construct
  source 1..32: mol_type = other DNA
aattactacaaattcttaccagtaatcccatc

SEQ ID NO: 61 - DNA, 32 bp, synthetic construct
  source 1..32: mol_type = other DNA
ttaagacgttgaaaacatagcgataacagtac

SEQ ID NO: 62 - DNA, 32 bp, synthetic construct
  source 1..32: mol_type = other DNA
tagaatccctgagaagagtcaataggaatcat

SEQ ID NO: 63 - DNA, 32 bp, synthetic construct
  source 1..32: mol_type = other DNA
cttttacacagatgaatatacagtaaacaatt

SEQ ID NO: 64 - DNA, 32 bp, synthetic construct
  source 1..32: mol_type = other DNA
tttaacgttcgggagaaacaataattttccct

SEQ ID NO: 65 - DNA, 32 bp, synthetic construct
  source 1..32: mol_type = other DNA
cgacaactaagtattagactttacaataccga

SEQ ID NO: 66 - DNA, 32 bp, synthetic construct
  source 1..32: mol_type = other DNA
ggatttagcgtattaaatcctttgttttcagg

SEQ ID NO: 67 - DNA, 32 bp, synthetic construct
  source 1..32: mol_type = other DNA
acgaaccaaaacatcgccattaaatggtggtt

SEQ ID NO: 68 - DNA, 32 bp, synthetic construct
  source 1..32: mol_type = other DNA
gaacgtggcgagaaaggaagggaacaaactat

SEQ ID NO: 69 - DNA, 32 bp, synthetic construct
  source 1..32: mol_type = other DNA
tagccctaccagcagaagataaaaacatttga

SEQ ID NO: 70 - DNA, 32 bp, synthetic construct
  source 1..32: mol_type = other DNA
cggccttgctggtaatatccagaacgaactga

SEQ ID NO: 71 - DNA, 32 bp, synthetic construct
  source 1..32: mol_type = other DNA
ctcagagccaccaccctcattttcctattatt

SEQ ID NO: 72 - DNA, 32 bp, synthetic construct
  source 1..32: mol_type = other DNA
ctgaaacaggtaataagttttaacccctcaga

SEQ ID NO: 73 - DNA, 32 bp, synthetic construct
  source 1..32: mol_type = other DNA
agtgtacttgaaagtattaagaggccgccacc

SEQ ID NO: 74 - DNA, 32 bp, synthetic construct
  source 1..32: mol_type = other DNA
gccaccactcttttcataatcaaaccgtcacc

SEQ ID NO: 75 - DNA, 32 bp, synthetic construct
  source 1..32: mol_type = other DNA
gtttgccacctcagagccgccaccgatacagg

SEQ ID NO: 76 - DNA, 32 bp, synthetic construct
  source 1..32: mol_type = other DNA
gacttgagagacaaaagggcgacaagttacca

SEQ ID NO: 77 - DNA, 32 bp, synthetic construct
  source 1..32: mol_type = other DNA
agcgccaaccatttgggaattagattattagc

SEQ ID NO: 78 - DNA, 32 bp, synthetic construct
  source 1..32: mol_type = other DNA
gaaggaaaataagagcaagaaacaacagccat

SEQ ID NO: 79 - DNA, 32 bp, synthetic construct
  source 1..32: mol_type = other DNA
gcccaataccgaggaaacgcaataggtttacc

SEQ ID NO: 80 - DNA, 32 bp, synthetic construct
  source 1..32: mol_type = other DNA
attatttaacccagctacaattttcaagaacg

SEQ ID NO: 81 - DNA, 32 bp, synthetic construct
  source 1..32: mol_type = other DNA
tattttgctcccaatccaaataagtgagttaa

SEQ ID NO: 82 - DNA, 32 bp, synthetic construct
  source 1..32: mol_type = other DNA
ggtattaagaacaagaaaaataattaaagcca

SEQ ID NO: 83 - DNA, 32 bp, synthetic construct
  source 1..32: mol_type = other DNA
taagtcctaccaagtaccgcactcttagttgc

SEQ ID NO: 84 - DNA, 32 bp, synthetic construct
  source 1..32: mol_type = other DNA
acgctcaaaataagaataaacaccgtgaattt

SEQ ID NO: 85 - DNA, 32 bp, synthetic construct
  source 1..32: mol_type = other DNA
aggcgttacagtagggcttaattgacaataga

SEQ ID NO: 86 - DNA, 32 bp, synthetic construct
  source 1..32: mol_type = other DNA
atcaaaatcgtcgctattaattaacggattcg

SEQ ID NO: 87 - DNA, 32 bp, synthetic construct
  source 1..32: mol_type = other DNA
ctgtaaatcataggtctgagagacgataaata

SEQ ID NO: 88 - DNA, 32 bp, synthetic construct
  source 1..32: mol_type = other DNA
cctgattgaaagaaattgcgtagacccgaacg

SEQ ID NO: 89 - DNA, 32 bp, synthetic construct
  source 1..32: mol_type = other DNA
acagaaatctttgaataccaagttccttgctt

SEQ ID NO: 90 - DNA, 32 bp, synthetic construct
  source 1..32: mol_type = other DNA
ttattaatgccgtcaatagataatcagaggtg

SEQ ID NO: 91 - DNA, 32 bp, synthetic construct
  source 1..32: mol_type = other DNA
agattagatttaaaagtttgagtacacgtaaa

SEQ ID NO: 92 - DNA, 32 bp, synthetic construct
  source 1..32: mol_type = other DNA
aggcggtcattagtctttaatgcgcaatatta

SEQ ID NO: 93 - DNA, 32 bp, synthetic construct
  source 1..32: mol_type = other DNA
gaatggctagtattaacaccgcctcaactaat

SEQ ID NO: 94 - DNA, 32 bp, synthetic construct
  source 1..32: mol_type = other DNA
ccgccagccattgcaacaggaaaaatattttt

SEQ ID NO: 95 - DNA, 32 bp, synthetic construct
  source 1..32: mol_type = other DNA
ccctcagaaccgccaccctcagaactgagact

SEQ ID NO: 96 - DNA, 32 bp, synthetic construct
  source 1..32: mol_type = other DNA
cctcaagaatacatggcttttgatagaaccac

SEQ ID NO: 97 - DNA, 32 bp, synthetic construct
  source 1..32: mol_type = other DNA
taagcgtcgaaggattaggattagtaccgcca

SEQ ID NO: 98 - DNA, 32 bp, synthetic construct
  source 1..32: mol_type = other DNA
caccagagttcggtcatagcccccgccagcaa

SEQ ID NO: 99 - DNA, 32 bp, synthetic construct
  source 1..32: mol_type = other DNA
tcggcattccgccgccagcattgacgttccag

SEQ ID NO: 100 - DNA, 32 bp, synthetic construct
  source 1..32: mol_type = other DNA
aatcaccaaatagaaaattcatatataacgga

SEQ ID NO: 101 - DNA, 32 bp, synthetic construct
  source 1..32: mol_type = other DNA
tcacaatcgtagcaccattaccatcgttttca

SEQ ID NO: 102 - DNA, 32 bp, synthetic construct
  source 1..32: mol_type = other DNA
atacccaagataacccacaagaataaacgatt

SEQ ID NO: 103 - DNA, 32 bp, synthetic construct
  source 1..32: mol_type = other DNA
atcagagaaagaactggcatgattttattttg

SEQ ID NO: 104 - DNA, 32 bp, synthetic construct
  source 1..32: mol_type = other DNA
ttttgtttaagccttaaatcaagaatcgagaa

SEQ ID NO: 105 - DNA, 32 bp, synthetic construct
  source 1..32: mol_type = other DNA
aggttttgaacgtcaaaaatgaaagcgctaat

SEQ ID NO: 106 - DNA, 32 bp, synthetic construct
  source 1..32: mol_type = other DNA
caagcaagacgcgcctgtttatcaagaatcgc

SEQ ID NO: 107 - DNA, 32 bp, synthetic construct
  source 1..32: mol_type = other DNA
aatgcagaccgtttttattttcatcttgcggg

SEQ ID NO: 108 - DNA, 32 bp, synthetic construct
  source 1..32: mol_type = other DNA
catatttagaaataccgaccgtgttacctttt

SEQ ID NO: 109 - DNA, 32 bp, synthetic construct
  source 1..32: mol_type = other DNA
aatggtttacaacgccaacatgtagttcagct

SEQ ID NO: 110 - DNA, 32 bp, synthetic construct
  source 1..32: mol_type = other DNA
taacctccatatgtgagtgaataaacaaaatc

SEQ ID NO: 111 - DNA, 32 bp, synthetic construct
  source 1..32: mol_type = other DNA
aaatcaatggcttaggttgggttactaaattt

SEQ ID NO: 112 - DNA, 32 bp, synthetic construct
  source 1..32: mol_type = other DNA
gcgcagagatatcaaaattatttgacattatc

SEQ ID NO: 113 - DNA, 32 bp, synthetic construct
  source 1..32: mol_type = other DNA
aacctaccgcgaattattcatttccagtacat

SEQ ID NO: 114 - DNA, 32 bp, synthetic construct
  source 1..32: mol_type = other DNA
attttgcgtctttaggagcactaagcaacagt

SEQ ID NO: 115 - DNA, 32 bp, synthetic construct
  source 1..32: mol_type = other DNA
ctaaaatagaacaaagaaaccaccagggttag

SEQ ID NO: 116 - DNA, 32 bp, synthetic construct
  source 1..32: mol_type = other DNA
gccacgctatacgtggcacagacaacgctcat

SEQ ID NO: 117 - DNA, 32 bp, synthetic construct
  source 1..32: mol_type = other DNA
gcgtaagagagagccagcagcaaaaaggttat

SEQ ID NO: 118 - DNA, 32 bp, synthetic construct
  source 1..32: mol_type = other DNA
ggaaatacctacattttgacgctcacctgaaa

SEQ ID NO: 119 - DNA, 32 bp, synthetic construct
  source 1..32: mol_type = other DNA
tatcaccgtactcaggaggtttagcggggttt

SEQ ID NO: 120 - DNA, 32 bp, synthetic construct
  source 1..32: mol_type = other DNA
tgctcagtcagtctctgaatttaccaggaggt

SEQ ID NO: 121 - DNA, 32 bp, synthetic construct
  source 1..32: mol_type = other DNA
ggaaagcgaccaggcggataagtgaataggtg

SEQ ID NO: 122 - DNA, 32 bp, synthetic construct
  source 1..32: mol_type = other DNA
tgaggcaggcgtcagactgtagcgtagcaagg

SEQ ID NO: 123 - DNA, 32 bp, synthetic construct
  source 1..32: mol_type = other DNA
tgcctttagtcagacgattggcctgccagaat

SEQ ID NO: 124 - DNA, 32 bp, synthetic construct
  source 1..32: mol_type = other DNA
ccggaaacacaccacggaataagtaagactcc

SEQ ID NO: 125 - DNA, 32 bp, synthetic construct
  source 1..32: mol_type = other DNA
acgcaaaggtcaccaatgaaaccaatcaagtt

SEQ ID NO: 126 - DNA, 32 bp, synthetic construct
  source 1..32: mol_type = other DNA
ttattacggtcagagggtaattgaatagcagc

SEQ ID NO: 127 - DNA, 32 bp, synthetic construct
  source 1..32: mol_type = other DNA
tgaacaaacagtatgttagcaaactaaaagaa

SEQ ID NO: 128 - DNA, 32 bp, synthetic construct
  source 1..32: mol_type = other DNA
ctttacagttagcgaacctcccgacgtaggaa

SEQ ID NO: 129 - DNA, 32 bp, synthetic construct
  source 1..32: mol_type = other DNA
gaggcgttagagaataacataaaagaacaccc

SEQ ID NO: 130 - DNA, 32 bp, synthetic construct
  source 1..32: mol_type = other DNA
tcattacccgacaataaacaacatatttaggc

SEQ ID NO: 131 - DNA, 32 bp, synthetic construct
  source 1..32: mol_type = other DNA
ccagacgagcgcccaatagcaagcaagaacgc

SEQ ID NO: 132 - DNA, 32 bp, synthetic construct
  source 1..32: mol_type = other DNA
agaggcataatttcatcttctgactataacta

SEQ ID NO: 133 - DNA, 32 bp, synthetic construct
  source 1..32: mol_type = other DNA
ttttagtttttcgagccagtaataaattctgt

SEQ ID NO: 134 - DNA, 32 bp, synthetic construct
  source 1..32: mol_type = other DNA
tatgtaaaccttttttaatggaaaaattacct

SEQ ID NO: 135 - DNA, 32 bp, synthetic construct
  source 1..32: mol_type = other DNA
ttgaattatgctgatgcaaatccacaaatata

SEQ ID NO: 136 - DNA, 32 bp, synthetic construct
  source 1..32: mol_type = other DNA
gagcaaaaacttctgaataatggaagaaggag

SEQ ID NO: 137 - DNA, 32 bp, synthetic construct
  source 1..32: mol_type = other DNA
tggattatgaagatgatgaaacaaaatttcat

SEQ ID NO: 138 - DNA, 32 bp, synthetic construct
  source 1..32: mol_type = other DNA
cggaattattgaaaggaattgaggtgaaaaat

SEQ ID NO: 139 - DNA, 32 bp, synthetic construct
  source 1..32: mol_type = other DNA
atcaacagtcatcatattcctgattgattgtt

SEQ ID NO: 140 - DNA, 32 bp, synthetic construct
  source 1..32: mol_type = other DNA
ctaaagcaagatagaacccttctgaatcgtct

SEQ ID NO: 141 - DNA, 32 bp, synthetic construct
  source 1..32: mol_type = other DNA
gccaacagtcaccttgctgaacctgttggcaa

SEQ ID NO: 142 - DNA, 32 bp, synthetic construct
  source 1..32: mol_type = other DNA
gaaatggattatttacattggcagacattctg

SEQ ID NO: 143 - DNA, 28 bp, synthetic construct
  source 1..28: mol_type = other DNA
tttttataagtatagcccggccgtcgag

SEQ ID NO: 144 - DNA, 36 bp, synthetic construct
  source 1..36: mol_type = other DNA
agggttgattttataaatcctcattaaatgatattc

SEQ ID NO: 145 - DNA, 36 bp, synthetic construct
  source 1..36: mol_type = other DNA
acaaacaattttaatcagtagcgacagatcgatagc

SEQ ID NO: 146 - DNA, 36 bp, synthetic construct
  source 1..36: mol_type = other DNA
agcaccgttttttaaaggtggcaacatagtagaaaa

SEQ ID NO: 147 - DNA, 36 bp, synthetic construct
  source 1..36: mol_type = other DNA
tacatacattttgacgggagaattaactacagggaa

SEQ ID NO: 148 - DNA, 36 bp, synthetic construct
  source 1..36: mol_type = other DNA
gcgcattattttgcttatccggtattctaaatcaga

SEQ ID NO: 149 - DNA, 36 bp, synthetic construct
  source 1..36: mol_type = other DNA
tatagaagttttcgacaaaaggtaaagtagagaata

SEQ ID NO: 150 - DNA, 36 bp, synthetic construct
  source 1..36: mol_type = other DNA
taaagtacttttcgcgagaaaactttttatcgcaag

SEQ ID NO: 151 - DNA, 36 bp, synthetic construct
  source 1..36: mol_type = other DNA
acaaagaattttattaattacatttaacacatcaag

SEQ ID NO: 152 - DNA, 36 bp, synthetic construct
  source 1..36: mol_type = other DNA
aaaacaaattttttcatcaatataatcctatcagat

SEQ ID NO: 153 - DNA, 36 bp, synthetic construct
  source 1..36: mol_type = other DNA
gatggcaattttaatcaatatctggtcacaaatatc

SEQ ID NO: 154 - DNA, 48 bp, synthetic construct
  source 1..48: mol_type = other DNA
aaaccctcttttaccagtaataaaagggattcaccagtcacacgtttt

SEQ ID NO: 155 - DNA, 32 bp, synthetic construct
  source 1..32: mol_type = other DNA
ccgaaatccgaaaatcctgtttgaagccggaa

SEQ ID NO: 156 - DNA, 32 bp, synthetic construct
  source 1..32: mol_type = other DNA
ccagcaggggcaaaatcccttataaagccggc

SEQ ID NO: 157 - DNA, 32 bp, synthetic construct
  source 1..32: mol_type = other DNA
gcataaagttccacacaacatacgaagcgcca

SEQ ID NO: 158 - DNA, 32 bp, synthetic construct
  source 1..32: mol_type = other DNA
gctcacaatgtaaagcctggggtgggtttgcc

SEQ ID NO: 159 - DNA, 32 bp, synthetic construct
  source 1..32: mol_type = other DNA
ttcgccattgccggaaaccaggcattaaatca

SEQ ID NO: 160 - DNA, 32 bp, synthetic construct
  source 1..32: mol_type = other DNA
gcttctggtcaggctgcgcaactgtgttatcc

SEQ ID NO: 161 - DNA, 32 bp, synthetic construct
  source 1..32: mol_type = other DNA
gttaaaattttaaccaataggaacccggcacc

SEQ ID NO: 162 - DNA, 32 bp, synthetic construct
  source 1..32: mol_type = other DNA
agacagtcattcaaaagggtgagaagctatat

SEQ ID NO: 163 - DNA, 32 bp, synthetic construct
  source 1..32: mol_type = other DNA
aggtaaagaaatcaccatcaatataatatttt

SEQ ID NO: 164 - DNA, 32 bp, synthetic construct
  source 1..32: mol_type = other DNA
tttcatttggtcaataacctgtttatatcgcg

SEQ ID NO: 165 - DNA, 32 bp, synthetic construct
  source 1..32: mol_type = other DNA
tcgcaaatggggcgcgagctgaaataatgtgt

SEQ ID NO: 166 - DNA, 32 bp, synthetic construct
  source 1..32: mol_type = other DNA
ttttaattgcccgaaagacttcaaaacactat

SEQ ID NO: 167 - DNA, 32 bp, synthetic construct
  source 1..32: mol_type = other DNA
aagaggaacgagcttcaaagcgaagatacatt

SEQ ID NO: 168 - DNA, 32 bp, synthetic construct
  source 1..32: mol_type = other DNA
ggaattactcgtttaccagacgacaaaagatt

SEQ ID NO: 169 - DNA, 32 bp, synthetic construct
  source 1..32: mol_type = other DNA
gaataaggacgtaacaaagctgctctaaaaca

SEQ ID NO: 170 - DNA, 32 bp, synthetic construct
  source 1..32: mol_type = other DNA
ccaaatcacttgccctgacgagaacgccaaaa

SEQ ID NO: 171 - DNA, 32 bp, synthetic construct
  source 1..32: mol_type = other DNA
ctcatcttgaggcaaaagaatacagtgaattt

SEQ ID NO: 172 - DNA, 32 bp, synthetic construct
  source 1..32: mol_type = other DNA
aaacgaaatgacccccagcgattattcattac

SEQ ID NO: 173 - DNA, 32 bp, synthetic construct
  source 1..32: mol_type = other DNA
cttaaacatcagcttgctttcgagcgtaacac

SEQ ID NO: 174 - DNA, 32 bp, synthetic construct
  source 1..32: mol_type = other DNA
tcggtttagcttgataccgatagtccaaccta

SEQ ID NO: 175 - DNA, 32 bp, synthetic construct
  source 1..32: mol_type = other DNA
tgagtttcgtcaccagtacaaacttaattgta

SEQ ID NO: 176 - DNA, 32 bp, synthetic construct
  source 1..32: mol_type = other DNA
ccccgatttagagcttgacggggaaatcaaaa

SEQ ID NO: 177 - DNA, 32 bp, synthetic construct
  source 1..32: mol_type = other DNA
gaatagccgcaagcggtccacgctcctaatga

SEQ ID NO: 178 - DNA, 32 bp, synthetic construct
  source 1..32: mol_type = other DNA
gagttgcacgagatagggttgagtaagggagc

SEQ ID NO: 179 - DNA, 32 bp, synthetic construct
  source 1..32: mol_type = other DNA
gtgagctagtttcctgtgtgaaatttgggaag

SEQ ID NO: 180 - DNA, 32 bp, synthetic construct
  source 1..32: mol_type = other DNA
tcatagctactcacattaattgcgccctgaga

SEQ ID NO: 181 - DNA, 32 bp, synthetic construct
  source 1..32: mol_type = other DNA
ggcgatcgcactccagccagctttgccatcaa

SEQ ID NO: 182 - DNA, 32 bp, synthetic construct
  source 1..32: mol_type = other DNA
gaagatcggtgcgggcctcttcgcaatcatgg

SEQ ID NO: 183 - DNA, 32 bp, synthetic construct
  source 1..32: mol_type = other DNA
aaataattttaaattgtaaacgttgatattca

SEQ ID NO: 184 - DNA, 32 bp, synthetic construct
  source 1..32: mol_type = other DNA
gcaaatatcgcgtctggccttcctggcctcag

SEQ ID NO: 185 - DNA, 32 bp, synthetic construct
  source 1..32: mol_type = other DNA
accgttctaaatgcaatgcctgagaggtggca

SEQ ID NO: 186 - DNA, 32 bp, synthetic construct
  source 1..32: mol_type = other DNA
tatattttagctgataaattaatgttgtataa

SEQ ID NO: 187 - DNA, 32 bp, synthetic construct
  source 1..32: mol_type = other DNA
tcaattcttttagtttgaccattaccagaccg

SEQ ID NO: 188 - DNA, 32 bp, synthetic construct
  source 1..32: mol_type = other DNA
cgagtagaactaatagtagtagcaaaccctca

SEQ ID NO: 189 - DNA, 32 bp, synthetic construct
  source 1..32: mol_type = other DNA
gaagcaaaaaagcggattgcatcagataaaaa

SEQ ID NO: 190 - DNA, 32 bp, synthetic construct
  source 1..32: mol_type = other DNA
tcagaagcctccaacaggtcaggatctgcgaa

SEQ ID NO: 191 - DNA, 32 bp, synthetic construct
  source 1..32: mol_type = other DNA
ccaaaatataatgcagatacataaacaccaga

SEQ ID NO: 192 - DNA, 32 bp, synthetic construct
  source 1..32: mol_type = other DNA
cattcaacgcgagaggcttttgcatattatag

SEQ ID NO: 193 - DNA, 32 bp, synthetic construct
  source 1..32: mol_type = other DNA
acgagtagtgacaagaaccggatataccaagc

SEQ ID NO: 194 - DNA, 32 bp, synthetic construct
  source 1..32: mol_type = other DNA
agtaatcttaaattgggcttgagagaatacca

SEQ ID NO: 195 - DNA, 32 bp, synthetic construct
  source 1..32: mol_type = other DNA
gcgaaacatgccactacgaaggcatgcgccga

SEQ ID NO: 196 - DNA, 32 bp, synthetic construct
  source 1..32: mol_type = other DNA
atacgtaaaagtacaacggagatttcatcaag

SEQ ID NO: 197 - DNA, 32 bp, synthetic construct
  source 1..32: mol_type = other DNA
caatgacactccaaaaggagccttacaacgcc

SEQ ID NO: 198 - DNA, 32 bp, synthetic construct
  source 1..32: mol_type = other DNA
aaaaaaggacaaccatcgcccacgcgggtaaa

SEQ ID NO: 199 - DNA, 32 bp, synthetic construct
  source 1..32: mol_type = other DNA
tgtagcattccacagacagccctcatctccaa

SEQ ID NO: 200 - DNA, 32 bp, synthetic construct
  source 1..32: mol_type = other DNA
gtaaagcactaaatcggaaccctagttgttcc

SEQ ID NO: 201 - DNA, 32 bp, synthetic construct
  source 1..32: mol_type = other DNA
agtttggagcccttcaccgcctggttgcgctc

SEQ ID NO: 202 - DNA, 32 bp, synthetic construct
  source 1..32: mol_type = other DNA
agctgattacaagagtccactattgaggtgcc

SEQ ID NO: 203 - DNA, 32 bp, synthetic construct
  source 1..32: mol_type = other DNA
actgcccgccgagctcgaattcgttattacgc

SEQ ID NO: 204 - DNA, 32 bp, synthetic construct
  source 1..32: mol_type = other DNA
cccgggtactttccagtcgggaaacgggcaac

SEQ ID NO: 205 - DNA, 32 bp, synthetic construct
  source 1..32: mol_type = other DNA
cagctggcggacgacgacagtatcgtagccag

SEQ ID NO: 206 - DNA, 32 bp, synthetic construct
  source 1..32: mol_type = other DNA
gtttgagggaaagggggatgtgctagaggatc

SEQ ID NO: 207 - DNA, 32 bp, synthetic construct
  source 1..32: mol_type = other DNA
ctttcatccccaaaaacaggaagaccggagag

SEQ ID NO: 208 - DNA, 32 bp, synthetic construct
  source 1..32: mol_type = other DNA
agaaaagcaacattaaatgtgagcatctgcca

SEQ ID NO: 209 - DNA, 32 bp, synthetic construct
  source 1..32: mol_type = other DNA
ggtagctaggataaaaatttttagttaacatc

SEQ ID NO: 210 - DNA, 32 bp, synthetic construct
  source 1..32: mol_type = other DNA
caacgcaatttttgagagatctactgataatc

SEQ ID NO: 211 - DNA, 32 bp, synthetic construct
  source 1..32: mol_type = other DNA
caataaatacagttgattcccaatttagagag

SEQ ID NO: 212 - DNA, 32 bp, synthetic construct
  source 1..32: mol_type = other DNA
tccatatacatacaggcaaggcaactttattt

SEQ ID NO: 213 - DNA, 32 bp, synthetic construct
  source 1..32: mol_type = other DNA
tacctttaaggtctttaccctgacaaagaagt

SEQ ID NO: 214 - DNA, 32 bp, synthetic construct
  source 1..32: mol_type = other DNA
caaaaatcattgctccttttgataagtttcat

SEQ ID NO: 215 - DNA, 32 bp, synthetic construct
  source 1..32: mol_type = other DNA
tttgccagatcagttgagatttagtggtttaa

SEQ ID NO: 216 - DNA, 32 bp, synthetic construct
  source 1..32: mol_type = other DNA
aaagattcagggggtaatagtaaaccataaat

SEQ ID NO: 217 - DNA, 32 bp, synthetic construct
  source 1..32: mol_type = other DNA
tttcaactataggctggctgaccttgtatcat

SEQ ID NO: 218 - DNA, 32 bp, synthetic construct
  source 1..32: mol_type = other DNA
ccaggcgcttaatcattgtgaattacaggtag

SEQ ID NO: 219 - DNA, 32 bp, synthetic construct
  source 1..32: mol_type = other DNA
cgcctgatggaagtttccattaaacataaccg

SEQ ID NO: 220 - DNA, 32 bp, synthetic construct
  source 1..32: mol_type = other DNA
tttcatgaaaattgtgtcgaaatctgtacaga

SEQ ID NO: 221 - DNA, 32 bp, synthetic construct
  source 1..32: mol_type = other DNA
atatattcttttttcacgttgaaaatagttag

SEQ ID NO: 222 - DNA, 32 bp, synthetic construct
  source 1..32: mol_type = other DNA
aataataaggtcgctgaggcttgcaaagactt

SEQ ID NO: 223 - DNA, 32 bp, synthetic construct
  source 1..32: mol_type = other DNA
cgtaacgatctaaagttttgtcgtgaattgcg

SEQ ID NO: 224 - DNA, 32 bp, synthetic construct
  source 1..32: mol_type = other DNA
acccaaatcaagttttttggggtcaaagaacg

SEQ ID NO: 225 - DNA, 32 bp, synthetic construct
  source 1..32: mol_type = other DNA
tggactcccttttcaccagtgagacctgtcgt

SEQ ID NO: 226 - DNA, 32 bp, synthetic construct
  source 1..32: mol_type = other DNA
tggtttttaacgtcaaagggcgaagaaccatc

SEQ ID NO: 227 - DNA, 32 bp, synthetic construct
  source 1..32: mol_type = other DNA
gccagctgcctgcaggtcgactctgcaaggcg

SEQ ID NO: 228 - DNA, 32 bp, synthetic construct
  source 1..32: mol_type = other DNA
cttgcatgcattaatgaatcggcccgccaggg

SEQ ID NO: 229 - DNA, 32 bp, synthetic construct
  source 1..32: mol_type = other DNA
attaagttcgcatcgtaaccgtgcgagtaaca

SEQ ID NO: 230 - DNA, 32 bp, synthetic construct
  source 1..32: mol_type = other DNA
tagatggggggtaacgccagggttgtgccaag

SEQ ID NO: 231 - DNA, 32 bp, synthetic construct
  source 1..32: mol_type = other DNA
acccgtcgtcatatgtaccccggtaaaggcta

SEQ ID NO: 232 - DNA, 32 bp, synthetic construct
  source 1..32: mol_type = other DNA
catgtcaagattctccgtgggaaccgttggtg

SEQ ID NO: 233 - DNA, 32 bp, synthetic construct
  source 1..32: mol_type = other DNA
tcaggtcacttttgcgggagaagcagaattag

SEQ ID NO: 234 - DNA, 32 bp, synthetic construct
  source 1..32: mol_type = other DNA
ctgtaatattgcctgagagtctggaaaactag

SEQ ID NO: 235 - DNA, 32 bp, synthetic construct
  source 1..32: mol_type = other DNA
caaaattaaagtacggtgtctggaagaggtca

SEQ ID NO: 236 - DNA, 32 bp, synthetic construct
  source 1..32: mol_type = other DNA
tgcaactaagcaataaagcctcagttatgacc

SEQ ID NO: 237 - DNA, 32 bp, synthetic construct
  source 1..32: mol_type = other DNA
tttttgcgcagaaaacgagaatgaatgtttag

SEQ ID NO: 238 - DNA, 32 bp, synthetic construct
  source 1..32: mol_type = other DNA
aaacagttgatggcttagagcttatttaaata

SEQ ID NO: 239 - DNA, 32 bp, synthetic construct
  source 1..32: mol_type = other DNA
actggataacggaacaacattattaccttatg

SEQ ID NO: 240 - DNA, 32 bp, synthetic construct
  source 1..32: mol_type = other DNA
acgaactagcgtccaatactgcggaatgcttt

SEQ ID NO: 241 - DNA, 32 bp, synthetic construct
  source 1..32: mol_type = other DNA
cgattttagaggacagatgaacggcgcgacct

SEQ ID NO: 242 - DNA, 32 bp, synthetic construct
  source 1..32: mol_type = other DNA
ctttgaaaagaactggctcattatttaataaa

SEQ ID NO: 243 - DNA, 32 bp, synthetic construct
  source 1..32: mol_type = other DNA
gctccatgagaggctttgaggactagggagtt

SEQ ID NO: 244 - DNA, 32 bp, synthetic construct
  source 1..32: mol_type = other DNA
acggctacttacttagccggaacgctgaccaa

SEQ ID NO: 245 - DNA, 32 bp, synthetic construct
  source 1..32: mol_type = other DNA
aaaggccgaaaggaacaactaaagctttccag

SEQ ID NO: 246 - DNA, 32 bp, synthetic construct
  source 1..32: mol_type = other DNA
gagaatagcttttgcgggatcgtcgggtagca

SEQ ID NO: 247 - DNA, 32 bp, synthetic construct
  source 1..32: mol_type = other DNA
acgttagtaaatgaattttctgtaagcggagt

SEQ ID NO: 248 - DNA, 28 bp, synthetic construct
  source 1..28: mol_type = other DNA
ttttcgatggcccactacgtaaaccgtc

SEQ ID NO: 249 - DNA, 36 bp, synthetic construct
  source 1..36: mol_type = other DNA
tatcagggttttcggtttgcgtattgggaacgcgcg

SEQ ID NO: 250 - DNA, 36 bp, synthetic construct
  source 1..36: mol_type = other DNA
gggagaggtttttgtaaaacgacggccattcccagt

SEQ ID NO: 251 - DNA, 36 bp, synthetic construct
  source 1..36: mol_type = other DNA
cacgacgtttttgtaatgggataggtcaaaacggcg

SEQ ID NO: 252 - DNA, 36 bp, synthetic construct
  source 1..36: mol_type = other DNA
gattgaccttttgatgaacggtaatcgtagcaaaca

SEQ ID NO: 253 - DNA, 36 bp, synthetic construct
  source 1..36: mol_type = other DNA
agagaatcttttggttgtaccaaaaacaagcataaa

SEQ ID NO: 254 - DNA, 36 bp, synthetic construct
  source 1..36: mol_type = other DNA
gctaaatcttttctgtagctcaacatgtattgctga

SEQ ID NO: 255 - DNA, 36 bp, synthetic construct
  source 1..36: mol_type = other DNA
atataatgttttcattgaatccccctcaaatcgtca

SEQ ID NO: 256 - DNA, 36 bp, synthetic construct
  source 1..36: mol_type = other DNA
taaatattttttggaagaaaaatctacgaccagtca

SEQ ID NO: 257 - DNA, 36 bp, synthetic construct
  source 1..36: mol_type = other DNA
ggacgttgtttttcataagggaaccgaaaggcgcag

SEQ ID NO: 258 - DNA, 36 bp, synthetic construct
  source 1..36: mol_type = other DNA
acggtcaattttgacagcatcggaacgaaccctcag

SEQ ID NO: 259 - DNA, 48 bp, synthetic construct
  source 1..48: mol_type = other DNA
cagcgaaattttaactttcaacagtttctgggattttgctaaactttt

SEQ ID NO: 260 - DNA, 62 bp, synthetic construct
  source 1..62: mol_type = other DNA
tataagtatagcccggccgtcgagagggttgaaaaaaaaaaaaaaaaaaaaaaaaaaaaaaa

SEQ ID NO: 261 - DNA, 62 bp, synthetic construct
  source 1..62: mol_type = other DNA
ataaatcctcattaaatgatattcacaaacaaaaaaaaaaaaaaaaaaaaaaaaaaaaaaaa

SEQ ID NO: 262 - DNA, 62 bp, synthetic construct
  source 1..62: mol_type = other DNA
aatcagtagcgacagatcgatagcagcaccgtaaaaaaaaaaaaaaaaaaaaaaaaaaaaaa

SEQ ID NO: 263 - DNA, 62 bp, synthetic construct
  source 1..62: mol_type = other DNA
taaaggtggcaacatagtagaaaatacatacaaaaaaaaaaaaaaaaaaaaaaaaaaaaaaa

SEQ ID NO: 264 - DNA, 62 bp, synthetic construct
  source 1..62: mol_type = other DNA
gacgggagaattaactacagggaagcgcattaaaaaaaaaaaaaaaaaaaaaaaaaaaaaaa

SEQ ID NO: 265 - DNA, 62 bp, synthetic construct
  source 1..62: mol_type = other DNA
gcttatccggtattctaaatcagatatagaagaaaaaaaaaaaaaaaaaaaaaaaaaaaaaa

SEQ ID NO: 266 - DNA, 62 bp, synthetic construct
  source 1..62: mol_type = other DNA
cgacaaaaggtaaagtagagaatataaagtacaaaaaaaaaaaaaaaaaaaaaaaaaaaaaa

SEQ ID NO: 267 - DNA, 62 bp, synthetic construct
  source 1..62: mol_type = other DNA
cgcgagaaaactttttatcgcaagacaaagaaaaaaaaaaaaaaaaaaaaaaaaaaaaaaaa

SEQ ID NO: 268 - DNA, 62 bp, synthetic construct
  source 1..62: mol_type = other DNA
attaattacatttaacacatcaagaaaacaaaaaaaaaaaaaaaaaaaaaaaaaaaaaaaaa

SEQ ID NO: 269 - DNA, 62 bp, synthetic construct
  source 1..62: mol_type = other DNA
ttcatcaatataatcctatcagatgatggcaaaaaaaaaaaaaaaaaaaaaaaaaaaaaaaa

SEQ ID NO: 270 - DNA, 62 bp, synthetic construct
  source 1..62: mol_type = other DNA
aatcaatatctggtcacaaatatcaaaccctcaaaaaaaaaaaaaaaaaaaaaaaaaaaaaa

SEQ ID NO: 271 - DNA, 62 bp, synthetic construct
  source 1..62: mol_type = other DNA
accagtaataaaagggattcaccagtcacacgaaaaaaaaaaaaaaaaaaaaaaaaaaaaaa

SEQ ID NO: 272 - DNA, 62 bp, synthetic construct
  source 1..62: mol_type = other DNA
cgatggcccactacgtaaaccgtctatcagggaaaaaaaaaaaaaaaaaaaaaaaaaaaaaa

SEQ ID NO: 273 - DNA, 62 bp, synthetic construct
  source 1..62: mol_type = other DNA
cggtttgcgtattgggaacgcgcggggagaggaaaaaaaaaaaaaaaaaaaaaaaaaaaaaa

SEQ ID NO: 274 - DNA, 62 bp, synthetic construct
  source 1..62: mol_type = other DNA
tgtaaaacgacggccattcccagtcacgacgtaaaaaaaaaaaaaaaaaaaaaaaaaaaaaa

SEQ ID NO: 275 - DNA, 62 bp, synthetic construct
  source 1..62: mol_type = other DNA
gtaatgggataggtcaaaacggcggattgaccaaaaaaaaaaaaaaaaaaaaaaaaaaaaaa

SEQ ID NO: 276 - DNA, 62 bp, synthetic construct
  source 1..62: mol_type = other DNA
gatgaacggtaatcgtagcaaacaagagaatcaaaaaaaaaaaaaaaaaaaaaaaaaaaaaa

SEQ ID NO: 277 - DNA, 62 bp, synthetic construct
  source 1..62: mol_type = other DNA
ggttgtaccaaaaacaagcataaagctaaatcaaaaaaaaaaaaaaaaaaaaaaaaaaaaaa

SEQ ID NO: 278 - DNA, 62 bp, synthetic construct
  source 1..62: mol_type = other DNA
ctgtagctcaacatgtattgctgaatataatgaaaaaaaaaaaaaaaaaaaaaaaaaaaaaa

SEQ ID NO: 279 - DNA, 62 bp, synthetic construct
  source 1..62: mol_type = other DNA
cattgaatccccctcaaatcgtcataaatattaaaaaaaaaaaaaaaaaaaaaaaaaaaaaa

SEQ ID NO: 280 - DNA, 62 bp, synthetic construct
  source 1..62: mol_type = other DNA
ggaagaaaaatctacgaccagtcaggacgttgaaaaaaaaaaaaaaaaaaaaaaaaaaaaaa

SEQ ID NO: 281 - DNA, 62 bp, synthetic construct
  source 1..62: mol_type = other DNA
tcataagggaaccgaaaggcgcagacggtcaaaaaaaaaaaaaaaaaaaaaaaaaaaaaaaa

SEQ ID NO: 282 - DNA, 62 bp, synthetic construct
  source 1..62: mol_type = other DNA
gacagcatcggaacgaaccctcagcagcgaaaaaaaaaaaaaaaaaaaaaaaaaaaaaaaaa

SEQ ID NO: 283 - DNA, 62 bp, synthetic construct
  source 1..62: mol_type = other DNA
aactttcaacagtttctgggattttgctaaacaaaaaaaaaaaaaaaaaaaaaaaaaaaaaa

SEQ ID NO: 284 - RNA, 18 nt, synthetic construct
  source 1..18: mol_type = other RNA
atatccttgtcgtatccc

SEQ ID NO: 285 - RNA, 18 nt, synthetic construct
  source 1..18: mol_type = other RNA
aataattgaattcttcaa

SEQ ID NO: 286 - RNA, 48 nt, synthetic construct
  source 1..48: mol_type = other RNA
aaaaaaaaaaaaaaaaaaaaaaaaaaaaaaatatccttgtcgtatccc

SEQ ID NO: 287 - RNA, 48 nt, synthetic construct
  source 1..48: mol_type = other RNA
aaaaaaaaaaaaaaaaaaaaaaaaaaaaaaaataattgaattcttcaa

SEQ ID NO: 288 - DNA, 46 bp, synthetic construct
  source 1..46: mol_type = other DNA
aaaaaaaaaaaaaaaaaaaaaaaaaaaaaattacgggatacgacaa

SEQ ID NO: 289 - DNA, 49 bp, synthetic construct; identical to SEQ ID NO: 16
  source 1..49: mol_type = other DNA

SEQ ID NO: 290 - DNA, 38 bp, synthetic construct; identical to SEQ ID NO: 8
  source 1..38: mol_type = other DNA

(19) United States
(12) Patent Application Publication
(10) Pub. No.: US 2026/0224732 A1
(43) Pub. Date: Aug. 6, 2026

(54) BIOPOLYMER PROTECTED OLIGONUCLEOTIDES FOR DRUG DELIVERY

(71) Applicant: THE ROYAL INSTITUTION FOR THE ADVANCEMENT OF LEARNING / MCGILL UNIVERSITY, Montreal (CA)

(72) Inventors: Christophe LACHANCE-BRAIS, Montreal (CA); Jathavan ASOHAN, Montreal (CA); Elizabeth GUETTLER, Montreal (CA); Matthew HARRINGTON, Montreal (CA); Mostafa RAMMAL, Montreal (CA); Chihyu YAO, Montreal (CA); Hanadi SLEIMAN, Montreal (CA)

(21) Appl. No.: 19/152,257

(22) PCT Filed: Jan. 31, 2024
(86) PCT No.: PCT/CA2024/050121

§ 371 Date: Jul. 30, 2025

Related U.S. Application Data
(60) Provisional application No. 63/482,407, filed on Jan. 31, 2023.

(51) Int. Cl. A61K 47/69; A61K 31/7088; A61K 47/54; C12N 15/11; C12N 15/113; C12N 15/115

(52) U.S. Cl. CPC A61K 47/6903; A61K 31/7088; A61K 47/549; C12N 15/111; C12N 15/113; C12N 15/115; C12N 2310/11; C12N 2310/3519; C12N 2320/32

(57) ABSTRACT

Conjugated nucleic acid molecules and methods are provided for delivery of a therapeutic oligonucleotide. The therapeutic oligonucleotide is bonded to a nucleic acid strand having a polyadenine segment that forms a supramolecular polymer with a thymine-mimicking compound. The supramolecular polymer is configured to dissociate to release the therapeutic oligonucleotide and grant it nuclease resistance. Nuclease resistance applies sequences adjacent to the polyadenine/thymine-mimicking assembly.

### FIELD

The present disclosure generally relates to drug delivery of oligonucleotides and in particular protecting oligonucleotides using biopolymers for drug delivery.

### BACKGROUND

Nucleic acids are one of the most biologically-relevant materials, both in their native impact on the human body, and in their potential for therapeutic application.¹ Recent advances in nucleic acids as therapeutics include the use of mRNAs in vaccines, such those against SARS-CoV-2, and in anti-tumour skin cancer injections.² However, one of the greatest barriers to mass production and distribution of therapeutic nucleic acids, particularly in RNA-based therapeutics, is the rapid degradation that these materials experience, when exposed to biological temperatures and conditions.¹ According to previous studies, the degradation of free nucleic acids in serum is protein-mediated, with over 300 different serum proteins contributing to the degradation process.³ The types of proteins responsible for nucleic acid breakdown include exonucleases, which cleave at the end of the strand (in particular, 3′ exonucleases, which start this process at the 3′ end), and endonucleases, which cleave in the middle of the strand.⁴ In the past, methods used to achieve nucleic acid stability include suspension in ionic liquids, encapsulation in lipid nanoparticles and liposomes, and formation of larger, complex, 3-dimensional structures, which take longer to degrade.^{1, 2, 5} Despite the variety in attempted stabilisation techniques, the degradation process remains on the hourly scale, for most nucleic acids studied.⁵ As such, improved techniques are needed to stabilize nucleic acids and for drug delivery.

### SUMMARY

In accordance with an aspect, there is provided a conjugated nucleic acid molecule for delivery of a therapeutic oligonucleotide, the conjugated nucleic acid molecule comprising a nucleic acid strand having a polyadenine segment that forms a supramolecular polymer with a thymine-mimicking compound; wherein the nucleic acid strand is bonded to the therapeutic oligonucleotide by covalent bonding or complementary base pairing; and wherein the supramolecular polymer is configured to dissociate to release the therapeutic oligonucleotide.

In another aspect, there is provided a method of protecting a therapeutic oligonucleotide from nuclease degradation for drug delivery, the method comprising: i) providing a biocompatible polymer having a polyadenine segment; ii) bonding the therapeutic oligonucleotide to the biocompatible polymer; and iii) contacting the bonded therapeutic oligonucleotide with a thymine-mimicking compound, under suitable conditions for the polyadenine segment to form a nucleic acid hydrogel, wherein the nucleic acid hydrogel protects the therapeutic oligonucleotide from nucleases.

In another aspect, there is provided use of the conjugated nucleic acid molecule as described herein for gene silencing.

In another aspect, there is provided a supramolecular polymer for delivery of a therapeutic oligonucleotide, the supramolecular polymer comprising a nucleic acid strand having a polyadenine segment and a biocompatible polymer grafted to a thymine-mimicking compound; wherein the nucleic acid strand is bonded to the therapeutic oligonucleotide by covalent bonding or complementary base pairing.

In another aspect, there is provided a method of protecting a therapeutic oligonucleotide from nuclease degradation for drug delivery, the method comprising: i) providing a nucleic acid strand having a polyadenine segment; ii) bonding the therapeutic oligonucleotide to the nucleic acid strand; and iii) contacting the bonded therapeutic oligonucleotide with a biocompatible polymer grafted to a thymine-mimicking compound under suitable conditions for the polyadenine segment to form a nucleic acid hydrogel, wherein the nucleic acid hydrogel protects the therapeutic oligonucleotide from nucleases.

In yet another aspect there is provided a kit comprising the conjugated nucleic acid molecule described herein and the thymine-mimicking compound described herein. In another aspect there is provided a kit comprising the nucleic acid strand as described herein and the biocompatible polymer grafted to the thymine-mimicking compound as described herein.

### DESCRIPTION OF THE FIGURES

Embodiments of devices, apparatus, methods, and kits are described throughout reference to the drawings.

FIG. 1 shows representative structure of a DNA hydrogel. a) Assembly of polyadenine DNA (dAₙ) with cyanuric acid (CA) to form a continuous hydrogen-bonded helicene structure (right), bringing together a phase-shifted triple helix that elongates into dA/CA supramolecular fibers. b) Hairy fibers of dA/CA motif crosslinking through their DNA overhangs. c) AFM image of diluted hydrogel of dA30C4v2 showing the multi-micron crosslinked fibers as the basis for the hydrogel assembly d) SEM image of the freeze-dried hydrogel showing the fiber bundles and pore structure. e) Image of a hydrogel of dA30C4v2 4 wt % (2 mM, CA 60 mM, Mag buffer) standing at an angle on a razor blade.

FIG. 2 shows PAGE results for CA=0 mM (left) and 20 mM (right) for strand A) dA30, B) dA30C4, C) rA15, D) rA15R7, E) R7rA15, F) Control.

FIG. 3 shows PAGE results for CA=0 mM (left) and 20 mM (right) for the gels of strand A) rA15, B) rA15R7, C) R7rA15, D) dA30C4v2, E) dA30C4v2′, F) da30C8 G) Control random.

FIG. 4 shows a degradation plot, fitted to first order rate equations, with [CA]=0 mM (blue circles) and 20 mM (orange squares). Rate constant, k, displayed as the slope., for A) dA30, B) dA30C4, C) rA15, D) rA15R7, E) R7rAa15, F) Control strands, performed in multiplate.

FIG. 5 shows the degradation plot fitted to first-order rate equations, the blue plot represents 0 mM [CA] and the orange one is 20 mM [CA] for A) control strand averages, B) rA15, C) rA15R7, D) R7rA15, E) dA30C4v2, F) dA30C4v2′, G) dA30C8, H) Control random.

FIG. 6 shows the CD spectrum of the A) RNA and B) DNA strands with different ratio between the polyadenine tail and the non-adenine sequence, in the presence of 20 mM of CA.

FIG. 7 shows the gel images of 1:1 DNA strands with polyA: non-A sequences in mouse serum, a) in CA 0 mM AND b) in CA 20 mM. Each sample is taken every 24 hours.

FIG. 8 shows the gel images of 1:2 DNA strands with polyA: non-A sequences in FBS, a) in CA 0 mM AND b) in CA 20 mM. Each sample is taken every 24 hours.

FIG. 9 shows the degradation plot of 1:2 DNA fitted to first-order rate equations, the blue plot represents 0 mM [CA] and the orange one is 20 mM [CA].

FIG. 10 shows the gel images of 1:3 DNA strands with polyA and complementary non-A sequences in FBS, a) in CA 0 mM AND b) in CA 20 mM. Each sample is taken every 24 hours.

FIG. 11 shows the degradation plot of 1:3 DNA fitted to first-order rate equations, the blue plot represents 0 mM [CA] and the orange one is 20 mM [CA].

FIG. 12 shows the gel images of 2:3 DNA strands with polyA and complementary non-A sequences in FBS, a) in CA OmM AND b) in CA 20 mM. Each sample is taken every 24 hours.

FIG. 13 shows the degradation plot of 2:3 DNA fitted to first-order rate equations, the blue plot represents 0 mM [CA] and the orange one is 20 mM [CA].

FIG. 14 shows the gel images of 3:2 DNA strand with polyA and non-A tail in FBS, a) in CA 0 mM AND b) in CA 20 mM. Each sample is taken every 24 hours. (C4V2

FIG. 15 shows the gel images of 3:2 DNA strand with polyA and non-A tail in FBS, a) in CA 0 mM AND b) in CA 20 mM. Each sample is taken every 24 hours. (C4V2)

FIG. 16 shows the gel images of 1:1 RNA strand with polyA and non-A tail in FBS, a) in CA 0 mM AND b) in CA 20 mM. Each sample is taken every 24 hours.

FIG. 17 shows the gel images of 1:1 RNA strand with polyA and non-A tail in FBS, a) in CA 0 mM AND b) in CA 20 mM. Each sample is taken every 24 hours.

FIG. 18 shows the gel images of 3:2 DNA strand with polyA and non-A tail in mouse serum, a) in CA 0 mM AND b) in CA 20 mM. Each sample is taken every 24 hours. (C4V2

FIG. 19 shows the gel images of ra15 in mouse serum, a) in CA 0 mM AND b) in CA 20 mM. Each sample is taken every 24 hours

FIG. 20 shows the gel images of DNA polyadenine strand in mouse serum, a) in CA 0 mM and b) in CA 20 mM. Each sample is taken every 24 hours. (dA30)

FIG. 21 shows the gel images of DNA polyadenine strand in mouse serum, a) in CA 0 mM and b) in CA 20 mM. Each sample is taken every 24 hours.

FIG. 22 shows the gel images of two DNA strand with polyA and complementary non-A tail in mouse serum in 1:1 ratio, a) in CA 0 mM AND b) in CA 20 mM. Each sample is taken every 24 hours. (1:1 C4v2 and C4v2′)

FIG. 23 shows the gel images of two DNA strand with polyA and complementary non-A tail in mouse serum in 1:1 ratio in mouse serum, a) in CA OmM and b) in CA 20 mM. Each sample is taken every 24 hours.

FIG. 24 shows the gel images of DNA polyadenine strand with i-motif in mouse serum, a) in CA 0 mM AND b) in CA 20 mM. Each sample is taken every 24 hours. (dA30 C4)

FIG. 25 shows the gel images of DNA polyadenine strand with i-motif in mouse serum, a) in CA 0 mM and b) in CA 20 mM. Each sample is taken every 24 hours.

FIG. 26 shows the gel images of RNA polyadenine strand with non A sequence on the 3′ end in mouse serum, a) in CA OmM and b) in CA 20 mM. Each sample is taken every 24 hours. (rA15R7)

FIG. 27 shows the gel images of RNA polyadenine strand with non A sequence on the 3′ end in mouse serum, a) in CA OmM and b) in CA 20 mM. Each sample is taken every 24 hours.

FIG. 28 shows the gel images of RNA polyadenine strand with non A sequence on the 5′ end in mouse serum, a) in CA OmM and b) in CA 20 mM. Each sample is taken every 24 hours. (R7 rA15)

FIG. 29 shows the gel images of RNA polyadenine strand with non A sequence on the 5′ end in mouse serum, a) in CA OmM and b) in CA 20 mM. Each sample is taken every 24 hours.

FIG. 30 shows the hexameric structure of poly A and CA assembly.

FIG. 31 shows the diagram of triple helixes formed by purely polyadenine strands and strands with protruding non-A ends

FIG. 32 shows that different parameters tune the mechanical properties of dA/CA hydrogels. a) Rheology of the variation length of the polyadenine region (dA15 (1400 μM), dA30 (700 μM), dA50 (420 μM), all CA 20 mM (dA:CA 1:1)). b) Schematic representation of the crosslinked dA/CA fiber structure with the blue wires representing DNA strands, the green spheres representing cyanuric acid (CA) and the red tails representing its possible sidechain. c) Rheology of crosslinking sequences (all 700 μM DNA and CA 20 mM): left double-stranded DNA; middle i-motif, through the assembly the assembly of d (cytosine) 4 or d (cytosine) 8 overhangs; right G-quadruplex through the assembly of d (guanine) 4 or d (guanine) 8 overhangs. d) Rheology of DNA concentration dependency for dA30C4v2 (CA 20, 40 and 60 mM). e) Effect of the nature of the sidechain of the cyanuric acid: CA and/or CAC2NH2 for dA15 (left) and dA30C8 (right). Single replicates are shown for clarity, for other replicates see FIGS. 46-50.

FIG. 33 shows self-healing behavior and thixotropy of the dA/CA hydrogels. a) Modulus as a response to shear for dA30-dsDNA13 at 700 μM. Around 1% shear strain, the G″ overtakes G′, indicating the yield point of the gel. b) Modulus in either a low shear regime (0.1%) or a high shear (100%) reversibly breaking and reforming the material. The rapid return to initial modulus indicates self-healing under 1 min. c) A dA30C: 700 μM CA 20 mM CAC2NH₂ 30 mM cut in two and stained either with GelRed or loading dye purple (up left panel) is joined back together and hung vertically (middle up image) or horizontally (bottom and right) with half of it dangling of a razor blade to demonstrate self-healing. d) Thixotropy of hydrogel dA₃₀C₄ 700 μM, CA 20 mM.

FIG. 34 shows multi-stimuli responsiveness of dA/CA hydrogels. Strand displacement stimuli a) reaction b) rheology (dA30C4 700 μM, CA 20 mM) and c) final state. pH stimuli d) reaction, e) rheology (dA30C4v2 700 μM, CA 20 mM) and f) basic state. Melamine stimuli h) reaction, i) rheology (dA₃₀-dsDNA₁₃ 700 μM, CA 20 mM) and j) final state. c) f) and j) are stained with Gel loading dye Purple.

FIG. 35 shows encapsulation, integration and release of DNA nanostructures within the dA/CA hydrogels. a) Representation of the DNA nanostructures tested. ssDNA has a 1 dA30 region, the rung has 6 (3 on each side) while the origami has 24 (12 on each side) b) Addition of pH 6 or pH 9 buffers to dA₃₀C₄ hydrogels having the DNA nanostructures either integrated or encapsulated. The hydrogels are placed on a coarse 0.2 μm filter which allows the nanostructures to pass through, but not the dA/CA fibers. c) Cy5 fluorescent imaging of DNA origami either with or without appended dA₃₀ sequences. After centrifugation, the hydrogel remains on the filter while the buffer flushes the pores and releases any encapsulated nanostructure. When the buffer is basic, the whole hydrogel is dissolved, and all its contents pass through the filter. Difference between the leakage and triggered release for d) single-stranded DNA, e) a DNA wireframe structure (tripodal rung of a DNA nanotube) and f) DNA origami. g) AFM of the DNA origami with dA₃₀ after pH triggered release showing the structure intact.

FIG. 36 shows release of antisense oligonucleotide (ASO) targeting the luciferase protein from assembled hydrogels in presence of HeLa cancer cells. a) Different methods by which the ASO (or the scramble sequence) are incorporated in the hydrogel; free, where the ASO is physically encapsulated, integrated, where the ASO has a dA₃₀ overhang that inserts it in the gel fibers, and complement, where the ASO is hybridized to a complementary overhang sequence on the hydrogel b) Composition of the hydrogel, with the component strands dA30C8 (700 μM), CA (20 mM) and CAC2NH₂ (30 mM) assembled in a pH buffer controlled by MES. c) The hydrogels are loaded in a syringe and injected onto plated Hela cells. d) Scheme of one of the mechanisms of action of ASO, created with BioRender.com. e) Luciferase protein and its catalyzed reaction, used as the reporter gene in this experiment. f) 24 h time point comparing the normalized luciferase intensity with and without the gel, and compared to controls. The without gel has no CA-R. i) 48 h timepoint comparison of the normalized luciferase signal from different incorporation methods. The without gel columns have CA-R.

FIG. 37 shows demonstration of the compatibility of the dA/CA motif and double-stranded DNA by UV-vis. a) UV-vis spectra of annealing of the dA30C4v2 strands (50 μM, CA 20 mM, Mag buffer, 80° C. to 20° C., 2° C./min) showing the double transition associated with its 2 constituent motifs. (double-stranded DNA at 68° C., theoretical Tm=69.5° C. by the IDT oligo analyzer online tool, dA/CA at 42° C.) b) Final circular dichroism spectra of the annealed strands showing the characteristic signal of the dA/CA motif, demonstrating its presence.

FIG. 38 shows circular dichroism spectra of i-motif and G-quadruplex forming strands (25 μM, Mag buffer) with or without the presence of CA. a-b) The i-motif strands show the appearance of the 295 nm band associated with i-motif only when CA is present. c) For the dA₃₀G4 strand, a faint increase in the 270 nm region as well as a partial cancelling of the dip in the 250-260 nm region suggest the formation of a G-quadruplex only when the CA is present. d) Without the addition of CA, the presence of a parallel G-quadruplex is clear for the dA₃₀G8 with the large positive peak at 260 nm followed by a dip in the 240-250 nm region. With CA, the signal is fainter, but the presence of the shoulder at 260 nm and the shift of the dip minima from the conventional 252 nm to 249 nm suggests the superposition of a parallel G-quadruplex signal with the dA/CA signal

FIG. 39 shows comparison of the rheological properties of different crosslinking sequences and their lengths (CA 20 mM, Mag buffer, 700 μM strands). Comparison between a) double-stranded DNA b) G-quadruplex and c) i-motif.

FIG. 40 shows rheology of dA₃₀C₈ 700 μM and CA 20 mM in the Mag and Serum buffers.

FIG. 41 shows SEM images of dA₃₀-dsDNA₁₃ 700 μM (CA 20 mM, Mag buffer) at different length scales.

FIG. 42 shows detailed single-layered rectangular DNA origami design where original staple strand 1-216 are marked at their 5′ end. Original staple strand sequences are listed in Table 9. Strand 175 contains a single Cy5 dye at its 3′ end.

FIG. 43 shows origami with poly-dA design. To add the poly-dA₃₀ overhangs, staple strands 100-111 and 205-216 were removed from the design and replaced with staple strands with dA₃₀ at their 3′ ends. The new staple sequences are listed in Table 10

FIG. 44 shows cell viability comparison with and without the presence of the hydrogel in the 24 h experiment. Free scramble and free ASO without gel are in a PBS buffer. CA+CAC2NH₂ column is with 20 mM of CA, 30 mM of CAC2NH₂ in Serum buffer, like the hydrogel environment. Error bars represent one standard deviation

FIG. 45 shows thermal stability of rheological properties of A) dA₃₀C₈ 700 μM, CA 20 mM, CAC2NH₂ 30 mM, Serum buffer from room temperature to 37° C., B) of duplicates of dA₃₀C₈ 2000 μM, CA 60 mM, CAC2NH₂ 90 mM, Serum buffer at 85° C.

FIG. 46 shows replicates of A) dA₃₀C₄ 700 μM, CA 20 mM Mag buffer at 25° C., B) dA₃₀C₈ 700 μM, CA 20 mM Mag buffer at 25° C., C) dA₃₀-dsDNA13 700 μM, CA 20 mM Mag buffer at 25° C., D) dA₃₀C₄v2 700 μM, CA 20 mM Mag buffer at 25° C., E) dA₃₀G4 700 μM, CA 20 mM Mag buffer at 25° C., F) dA₃₀G₈ 700 μM, CA 20 mM Mag buffer at 25° C.

FIG. 47 shows replicates of dA₁₅ 700 μM, CA or CAC2NH₂ 20 mM Mag buffer at 25° C. The first replicate of CA 20 mM shows some slippage, the values used for the moduli at 1 Hz are interpolated linearly.

FIG. 48 shows replicates of dA₃₀C₈ 700 μM, CA 60 mM, CAC2NH₂ 90 mM Serum buffer at 25° C. Value at 10 Hz for the second replicate is interpolated linearly for the calculation of the mean presented in the manuscript.

FIG. 49 shows mean of moduli of dA₃₀ with different crosslinking regions at 700 μM, CA 20 mM and Mag buffer at 25° C., 1 Hz.

FIG. 50 shows replicates of dA₁₅ (1400 μM), dA₃₀ (700 μM) and dA₅₀ (420 μM) CA 20 mM Mag buffer at 25° C., 1 Hz.

FIG. 51 shows viscosity versus time in the Shear thinning experiment (hydrogel dA₃₀C₄ 700 μM, CA 20 mM, Mag buffer).

FIG. 52 shows variation in stoechoiometric ratios for dA₃₀C₈ 700 μM Serum buffer at 25° C., showing a similar value when there is excess CA for dA.

FIG. 53 shows variation in stoechoiometric ratios for dA₃₀C₈ 2 mM Serum buffer at 25° C., showing a lower than potential value when there is excess dA for CA:CAC2NH₂.

FIG. 54 shows degradation experiments using the exonuclease Exo I and the endonuclease DNAse I on the sequences of dA₃₀ and DNA 1:1. The enzymes were used at 1U of concentration and the DNA at 25M.

FIG. 55 shows degradation experiments on double-stranded DNA (dsDNA) attached to a polyA tail with and without cyanuric acid. a) The results are visualized on a native polyacrylamide gel of 8% and the ladder is the ultralow range DNA ladder from Thermo Fisher. Each strand in the DNA sequence was used at 12.5 μM, and they were annealed together in a 1:1 ratio before having the CA and enzymes added to them. b) Half-lives both with and without CA of the double-stranded construct.

FIG. 56 shows degradation experiments on the DNA 1:1 sequence changing the sidechain of the CA molecule. Different mixtures of CA-R with different R groups can control the rate of degradation of the associated sequence. The order of stabilization follows the bundling ability of the CA-R added. In the best scenario, the stability of DNA 1:1 is regenerated to the level of stability of dA₃₀ itself, lasting a week without observable degradation. a) Representative denaturing polyacrylamide gel electrophoresis experiments, b) Half-lives with the difference CA-R mixtures extracted from triplicates.

FIG. 57 shows viability and morphology of epithelial cells with various mixtures of CA and CA-R derivatives. a) Bar graph of the viability of the cells at different times and in different conditions. For each set of 6 bars from left to right: i) CA 20 mM, pH6, ii) CA 20 mM+CAC2NH2 30 mM, pH6, iii) CA 20 mM+CAC5NH2 18 mM, pH6, iv) CA 20 mM+CAC6NH_{2 12} mM, pH6, v) pH7,2, iv) pH6. b) Morphology of the cells on day 5 (120 h), as seen by confocal microscopy. Magnification of 10×, scale bar=100 μm, dapi in blue and factin in red.

FIG. 58 shows degradation experiments on the ratiometric sequences, named after the ratio of their polyA: non-polyA sequence. Conditions are in Serum buffer supplemented with 10% FBS either with or without cyanuric acid (CA) and with or without an additional 25 μM of dA₃₀ added.

### DETAILED DESCRIPTION

The present disclosure provides conjugated molecules and methods for stabilizing both DNA and RNA strands with polyA regions and thymine-mimicking compounds. By forming long, supramolecularly-assembled polymers (for example hydrogels crosslinked by the polyA regions) using the polyA regions and thymine-mimicking compounds, a strategy for achieving nucleic acid stability and protection against nucleases is provided. It is believed that these supramolecular structures limit the access of exo- and endonucleases to the DNA and RNA strands, thereby stabilising the it from standard degradation processes.

### Conjugated Molecules

In accordance with the present disclosure, conjugated molecules are provided for the delivery of therapeutic oligonucleotides. As used herein, “therapeutic oligonucleotides” refers to nucleic acid molecules, whether DNA or RNA, modified or unmodified, or combination thereof, that when administered in a subject results in a therapeutic or beneficial effect on the subject. The conjugated molecules improves the stability of the therapeutic oligonucleotides for administration and/or diagnosis. In some embodiments, a conjugated nucleic acid molecule comprises a nucleic acid strand having a polyadenine segment that forms a supramolecular polymer with a thymine-mimicking compound. As used herein, a “thymine-mimicking compound” refers to a compound (preferably non-toxic, biocompatible, and/or bio-tolerable) capable of hydrogen bonding with an adenine nucleotide base of the polyadenine segment, thereby triggering the formation of a supramolecular structure. In one embodiment, the thymine-mimicking compound is cyanuric acid, xanthine, barbituric acid, uric acid, or a derivative thereof. In one embodiment, the thymine-mimicking compound is one or more of cyanuric acid, xanthine, barbituric acid, uric acid, a derivative of cyanuric acid, a derivative of xanthine, a derivative of barbituric acid, and a derivative of uric acid. Preferably, the thymine-mimicking compound is cyanuric acid, or a derivative thereof. Displacement of the thymine-mimicking compound, or loss of hydrogen bonding between the thymine-mimicking compound and adenines results in dissociation or loss of the supramolecular polymer structure, thereby releasing the conjugated nucleic acid molecule carrying the therapeutic oligonucleotide.

In some embodiments, the therapeutic oligonucleotide is covalently bonded to the polyadenine segment, or bonded via an optional linker sequence, and the conjugated nucleic acid molecule is represented by formula (Ia) and (Ib) below:

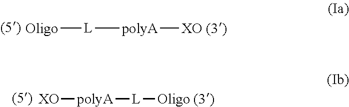

where Oligo is the therapeutic oligonucleotide; L is an optional linker sequence, polyA is the polyadenine segment, XO is an optional crosslinking overhang segment, (5′) indicates the 5′ end of the conjugated nucleic acid molecule; (3′) indicates the 3′ end of the conjugated nucleic acid molecule; and “—” is a nucleotide linkage. In some embodiments, the overhang segment comprises single stranded DNA. In other embodiments, the overhang segment comprises double stranded DNA. In yet other embodiments, the overhang segment comprises partly single stranded DNA and partly double stranded DNA.

For improved stability and formation of the supramolecular polymer, the relative length of the polyadenine segment to the rest of the conjugated nucleic acid molecule play an important role. The longer the therapeutic oligonucleotide sequence, the more adenines are needed to ensure sufficient interaction with the thymine-mimicking compound to form a supramolecular polymer. The present inventors have determined that a particular ratio of adenine nucleotides to non-adenine nucleotides in the conjugated nucleic acid molecule, including nucleotides of the therapeutic oligonucleotide, is needed to ensure proper supramolecular polymer formation. In some embodiments, the ratio of adenine nucleotides to non-adenine nucleotides in the conjugated nucleic acid molecule is between 1:1 and 2:3.

In some embodiments, the therapeutic oligonucleotide is complementary base paired to the an overhang segment of the conjugated nucleic acid molecule. The overhang segment having a nucleic acid sequence complementary to the therapeutic oligonucleotide, and the conjugated nucleic acid molecule is represented by formula (IIa) and (IIb) below:

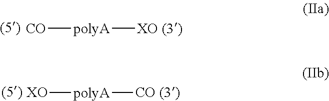

where CO is the overhang segment having a nucleic acid sequence complementary to the therapeutic oligonucleotide, polyA is the polyadenine segment, XO is an optional crosslinking overhang segment, (5′) indicates the 5′ end of the conjugated nucleic acid molecule; (3′) indicates the 3′ end of the conjugated nucleic acid molecule; and “—” is a nucleotide linkage.

In some embodiments, the polyadenine segment is a DNA polyadenine segment, while in other embodiments the polyadenine segment is a RNA polyadenine segment. In some embodiments, the polyadenine segment comprises modified nucleotides. Exemplary modified nucleotides include, but are not limited to, RNA nucleotides where a —OH group is substituted with methoxy Linear Nucleic Acid (LNA), Fluoro Arabinose Nucleic Acid (FANA), or Peptidic Nucleic Acid (PNA). In some embodiments, the therapeutic oligonucleotide is DNA, while in other embodiments the therapeutic oligonucleotide is RNA. In one embodiment, both the polyadenine segment and the therapeutic oligonucleotide are DNA. In one embodiment, both the polyadenine segment and the therapeutic oligonucleotide are RNA.

In one embodiment, the therapeutic oligonucleotide is an antisense oligonucleotide. In one embodiment, the therapeutic oligonucleotide is a therapeutic aptamer. In some embodiments, the therapeutic oligonucleotide is a RNA. In one embodiment, the therapeutic oligonucleotide is a mRNA. In one embodiment, the therapeutic oligonucleotide is a small interfering RNA (siRNA). In one embodiment, the therapeutic oligonucleotide is a microRNA (miRNA). In one embodiment, the therapeutic oligonucleotide is a short hairpin RNA (shRNA).

### Modifications to Supramolecular Polymer

A number of parameters are available for modification or customization of the supramolecular polymer properties, such as stiffness, self-healing properties, thixotropy, and stimuli-responsiveness. In some embodiment, the polyadenine segment comprises at least 10 adenine nucleotides, at least 15 adenine nucleotides, at least 20 adenine nucleotides, at least 25 adenine nucleotides, at least 30 adenine nucleotides, at least 40 adenine nucleotides, or at least 50 adenine nucleotides. In one embodiment, the polyadenine segment comprises preferably at least 15 adenine nucleotides. In some embodiments, the polyadenine segment comprises between 10 to 300 adenine nucleotides, between 10 to 50 adenine nucleotides, between 10 to adenine nucleotides, between 15 to 50, or preferably between 15 to 30 adenine nucleotides.

Crosslinking may also be present in the supramolecular polymer. In some embodiments, the nucleic acid strand comprises a crosslinking overhang segment for base pairing with a complementary crosslinking overhang segment of a biocompatible polymer to form the supramolecular polymer. As used herein, a “biocompatible polymer” refers to a polymer material that is not toxic, injurious, or physiologically reactive and does not cause immunological rejection with a living tissue or subject. Exemplary biocompatible polymers include, but are not limited to: alginate, chitosan, agarose, hyaluronic acid, gelatine, polyacrylamide, peg, or mixtures thereof such as gelatin-alginate.

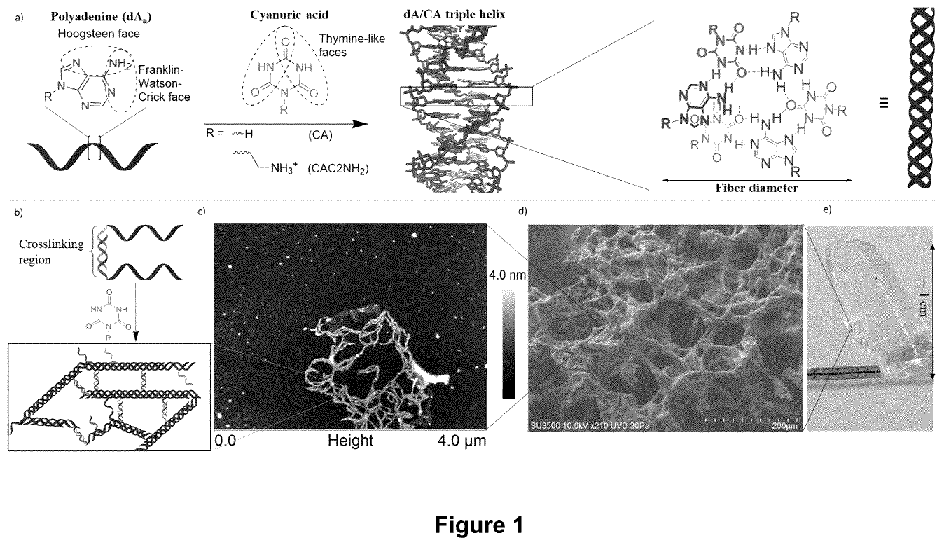

In some embodiments, the biocompatible polymer is a DNA hydrogel, and the crosslinking overhang segments base pairs providing crosslinking structures in the DNA hydrogel. (See FIG. 1) In one embodiment, the crosslinking structure comprises a double stranded DNA segment. In another embodiment, the crosslinking structure comprises an i-motif. In yet another embodiments, the crosslinking structure comprises a G-quadruplex. In some embodiments, the crosslinking structures of the DNA hydrogel comprise one or more of double stranded DNA segments, i-motif, and G-quadruplex.

In some embodiments, the supramolecular polymer comprises one or more types of polymers. In one embodiment, the supramolecular polymer is a double network mixture. For example a chitosan double network with the polyadenine/cyanuric acid hydrogel. In another embodiment, the supramolecular polymer comprises a biocompatible polymer grafted to the conjugated nucleic acid molecule or the thymine-mimicking compound.

In some embodiments, the conjugated nucleic acid molecule has a flanking segment for providing further protection of the therapeutic oligonucleotide against nucleases. In one embodiment, the flanking segment is located adjacent to the polyadenine segment, on the 5′ end or the 3′ end. In one embodiment, the linker is a flanking segment. In one embodiment, the flanking segment is located within the polyadenine segment. The flanking segment may be an internal hairpin or a double stranded DNA region.

To improve the stability and formation of the supramolecular polymer where there are more non-adenine nucleotides relative to adenine nucleotides, free polyadenine nucleic acid strands are provided. As used herein, “free polyadenine nucleic acid strands” refer to non-conjugated nucleic acid strands consisting of adenine bases. The conjugated nucleic acid molecule and the free polyadenine nucleic acid strands are contacted with a thymine-mimicking compound, under suitable conditions for the polyadenine segment and the free polyadenine nucleic acid strand to form a nucleic acid hydrogel. In embodiments where free polyadenine nucleic acid strands are added, the ratio of adenine nucleotides to non-adenine nucleotides in the conjugated nucleic acid molecule is between 1:1 to 1:3. In one embodiment, the free polyadenine nucleic acid strand comprises DNA or RNA adenine nucleotides or modified nucleotides thereof. In some embodiment, the free polyadenine nucleic acid strand comprises at least 10 adenine nucleotides, at least 15 adenine nucleotides, at least 20 adenine nucleotides, at least 25 adenine nucleotides, at least 30 adenine nucleotides, at least 40 adenine nucleotides, or at least 50 adenine nucleotides. In one embodiment, the polyadenine segment comprises preferably at least adenine nucleotides. In some embodiments, the polyadenine segment comprises between 10 to 300 adenine nucleotides, between 10 to 50 adenine nucleotides, between 10 to 30 adenine nucleotides, between 15 to 50, or preferably between 15 to 30 adenine nucleotides.

Methods of Protecting Therapeutic Oligonucleotide with Polymers

The present disclosure also provides for method of protecting a therapeutic oligonucleotide from nuclease degradation, which is useful for drug delivery. A biocompatible polymer having a polyadenine segment is provided, and bonded to a therapeutic oligonucleotide. In some embodiments, bonding the therapeutic oligonucleotide comprises covalent bonding the 5′ or 3′ end of the therapeutic oligonucleotide to the biocompatible polymer. In other embodiments, the biocompatible polymer comprises a complementary overhang segment having a nucleic acid sequence complementary to the therapeutic oligonucleotide, such that the therapeutic oligonucleotide base pairs to the biocompatible polymer. The bonded therapeutic oligonucleotide is then combined with a thymine-mimicking compound under suitable conditions for the polyadenine segment of the biocompatible polymer to form a nucleic acid hydrogel. Suitable conditions for the formation of the nucleic acid hydrogel include a suitable pH environment for the nitrogens of the thymine-mimicking compound to be protonated, and thereby available for hydrogen bonding. A suitable pH is based on the pKa of the thymine-mimicking compound. The present inventors have discovered that the formation of the nucleic acid hydrogel protects the therapeutic oligonucleotide from nucleases.

In some embodiments, the biocompatible polymer is a nucleic acid hydrogel. In some embodiments, the biocompatible polymer is covalently grafted with the polyadenine segment. In some embodiments, the biocompatible polymer comprises one or more different types of polymers. In some embodiments, various copies of bonded therapeutic oligonucleotide are combined with the thymine-mimicking compound to form the nucleic acid hydrogel. In some embodiments, various copies of bonded therapeutic oligonucleotide having different oligonucleotide sequences are combined with the thymine-mimicking compound to form the nucleic acid hydrogel. In some embodiments, the bonded therapeutic oligonucleotide and another biocompatible polymer having polyadenine segment are combined with the thymine-mimicking compound to form the nucleic acid hydrogel.

In some embodiments, forming the biocompatible polymer forms a double network mixture with another biocompatible polymer. In some embodiments, forming the nucleic acid hydrogel comprises forming a double network mixture with the nucleic acid hydrogel. In one embodiment, the biocompatible polymer comprises a crosslinking overhang segment for base pairing with a complementary crosslinking overhang segment of a further nucleic acid hydrogel. The nucleic acid hydrogel is formed by contacting the biocompatible polymer with said further nucleic acid hydrogel under suitable conditions for base pairing of the second overhang segment with the complementary crosslinking overhang segment.

The nucleic acid hydrogel is dissociated by introducing a stimulus, in order to release the bonded therapeutic oligonucleotide. In some embodiments, the nucleic acid hydrogel is subjected to increases in pH, deprotonating the thymine-mimicking compound which results in loss of hydrogen bonding with the polyadenine segment and thereby unraveling the nucleic acid hydrogel. In the context of drug delivery, administration to a target cite results in exposure to a physiological pH that is higher than the suitable pH for nucleic acid hydrogel formation, resulting in dissolution of the hydrogel and release of the therapeutic oligonucleotide. In some embodiments, a molecule that compete with the thymine-mimicking compound is introduced to dissociate the nucleic acid hydrogel. Molecules capable of competing with the thymine-mimicking compound for hydrogen bonding with the polyadenine segment include a polythymine stand, melamine or derivatives thereof.

### Thymine-Mimicking Compounds and Modification Thereof

In some embodiments, the thymine-mimicking compound is selected from the group consisting of cyanuric acid, xanthine, barbituric acid, and uric acid, or a combination thereof. In some embodiments, the thymine-mimicking compound is a derivative of cyanuric acid, a derivative of xanthine, a derivative of barbituric acid, or a derivative of uric acid. In one embodiment, thymine-mimicking compound is cyanuric acid (CA) or a derivative thereof. As used herein, a “a derivative of cyanuric acid, a derivative of xanthine, a derivative of barbituric acid, or a derivative of uric acid” refers to cyanuric acid, xanthine, barbituric acid, or uric acid in a multimer form (such as dimer or trimer), or that is substituted with a side chain that does not interrupt with the capacity of these thymine-mimicking compounds to hydrogen bond with the polyadenine segment to form nucleic acid hydrogels. In one embodiment, the thymine-mimicking compound comprise a combination of cyanuric acid and one or more cyanuric acid derivative. In one embodiment, the derivative of cyanuric acid has a side chain conjugated to one of the nitrogens. Exemplary side chains include, but a not limited to a C₂-C₆ side chain optionally substituted with an alcohol, an amine, an ester, an amide, or a guanidinium. In one embodiment, the side chain is —(CH₂)ₙ—NH₂, where n is 2 to 6. In one embodiment, the cyanuric acid derivative is:

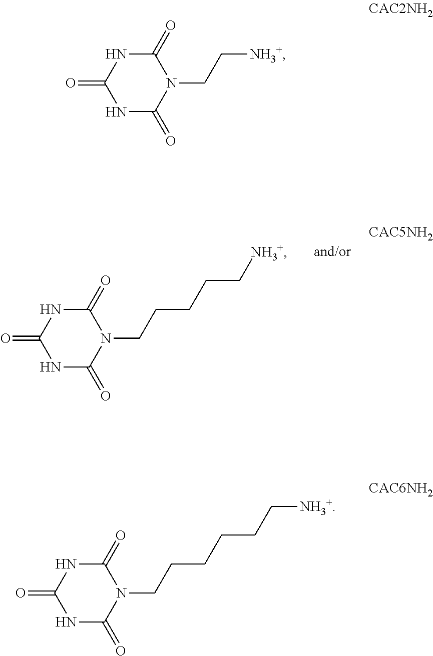

The thymine-mimicking compound may be further modified by grafting the biocompatible polymer to the thymine-mimicking compound instead of the polyadenine segment. In some embodiments, a supramolecular polymer for delivery of a therapeutic oligonucleotide is provided, where the supramolecular polymer is made from a nucleic acid strand having a polyadenine segment and a thymine-mimicking compound with a biocompatible polymer grafted thereon. The nucleic acid strand is bonded to the therapeutic oligonucleotide by covalent bonding or complementary base pairing as described herein. When

In some embodiments, methods of protecting a therapeutic oligonucleotide from nuclease degradation for drug delivery comprise providing a nucleic acid strand having a polyadenine segment, and bonding the therapeutic oligonucleotide to the nucleic acid strand. This bonded therapeutic oligonucleotide is combined with a modified thymine-mimicking compound having a biocompatible polymer grafted thereon for the polyadenine segment to form a nucleic acid hydrogel with the modified thymine-mimicking compound. The nucleic acid hydrogel protects the therapeutic oligonucleotide from nucleases.

### Therapeutic Uses and Kits

The therapeutic oligonucleotide described herein can be a DNA oligonucleotide or a RNA oligonucleotide. In one embodiment, the therapeutic oligonucleotide is an antisense oligonucleotide. In some embodiments, the conjugated nucleic acid molecule described herein is used for gene silencing. In one embodiment, the therapeutic oligonucleotide is a therapeutic aptamer. In one embodiment, the therapeutic oligonucleotide is a mRNA, small interfering RNA (siRNA), microRNA (miRNA), or short hairpin RNA (shRNA).

The present disclosure also provides for kits for preparing the supramolecular polymer described herein. In one embodiment, the kit comprises 1) a conjugated nucleic acid molecule described herein and 2) a thymine-mimicking compound. In another embodiment, the kit comprises 1) a nucleic acid strand having a polyadenine segment and bonded to the therapeutic oligonucleotide, and 2) a modified thymine-mimicking compound having a biocompatible polymer grafted thereon.

### EXAMPLES

The following examples illustrate certain embodiments addressing specific design requirements and are not intended to limit the embodiments described elsewhere in this disclosure.

### Example 1A-Protecting Oligonucleotides from Nucleases

Because polymers only form in strands containing polyadenine (“polyA”) regions, by using polyA sequences alongside polyA sequences with modified ends, it is possible to elucidate whether cyanuric acid (“CA”) stabilises DNA and RNA strands against degradation by exo-vs endonucleases. These sequences may be compared alongside a sequence without polyA, to further show that it is the cord-like structure, formed between the CA and nucleic acid (“NA”) strands, and crosslinked by the polyA stretches, which stabilises these strands.^{6, 7}

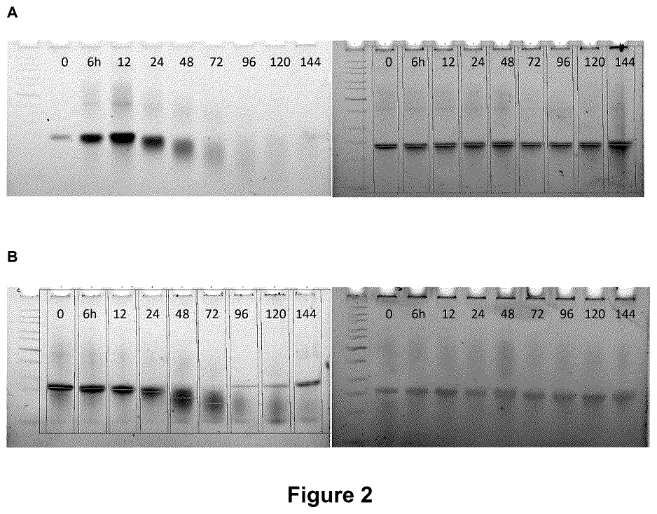

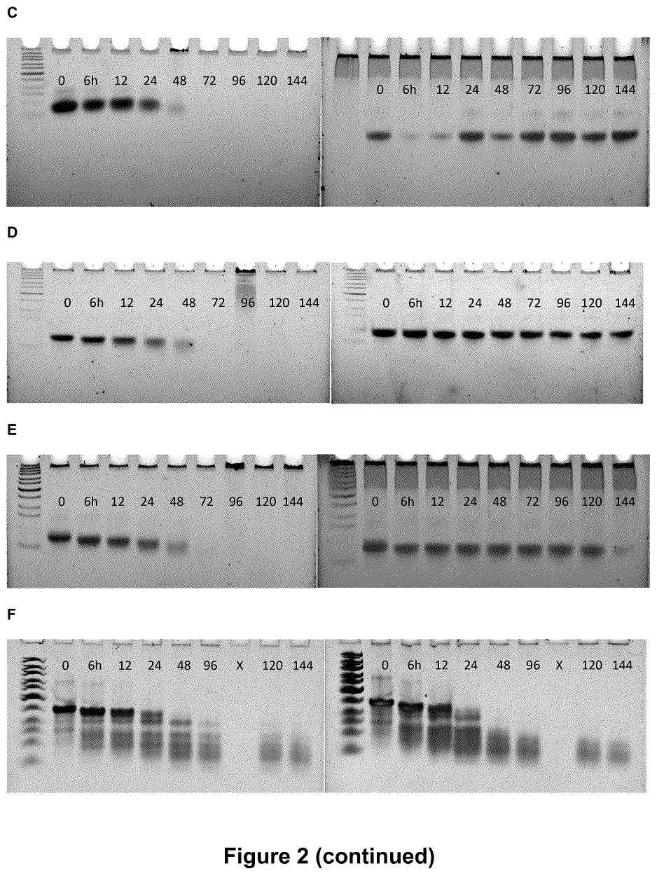

The sequences were ordered from Integrated DNA Technologies (IDT) and used as is. They include different positions and lengths of polyadenine regions with relation to the desired protected sequences. For each strand analysed, 60 μL nucleic acid stock solutions (strand concentration of 25 μM) were prepared in L15 media (fortified with 15% fœtal bovine serum and 20 mM MES, pH 6.2-6.4) or a Serum media (fortified with 10% foetal bovine serum, NaCl 140 mM, KCl 5 mM, Ca(NO₃)₂ 2.5 mM, MgCl₂ 1.5 mM, MES 10 mM, pH 5.7-6.2). They either contained containing 0 mM cyanuric acid (CA) and 20 mM CA. A 5 μL sample was aliquoted from each, marking the 0 h time-point, to which 5 μL urea, 8M was added. The sample then was stored at −20° C. The stocks were incubated at 37° C., with all further measurements taken in a similar fashion, at their respective time-points. Samples were analysed by denaturing polyacrylamide gel electrophoresis (PAGE), 20% for RNAs and 15% for DNAs, 100V for 1 h in 1×TBE. The gels were stained with SybrGold from ThermoFisher, then imaged on a BioRad ChemiDoc XRS+ imaging system, from which the strongest bands were selected (FIG. 2) using the BioRad Image Lab software.

TABLE 1
Strands, lengths and corresponding sequences analysed
in serum stability assays.

| Name | Sequence | Length | SEQ ID NO |
|---|---|---|---|
| rA15 | AAAAAAAAAAAAAAA | 15 | 1 |
| rA15R7 | GCTACTGAAAAAAAAAAAAAAA | 22 | 2 |
| R7rA15 | AAAAAAAAAAAAAAAGTCATCG | 22 | 3 |
| rA7-loop12-rA7 | AAAAAAAGCCGTTGACGGCAAAAAAA | 52 | 4 |
| dA15 | AAAAAAAAAAAAAAA | 15 | 5 |
| dA30 | AAAAAAAAAAAAAAAAAAAAAAAAAAAAAA | 30 | 6 |
| dA30C4-v2 | AAAAAAAAAAAAAAAAAAAAAAAAAAAAAA | 54 | 7 |
| | CCCCATTCTACTTGAGAGAGCGAC | | |
| dA30C8 | AAAAAAAAAAAAAAAAAAAAAAAAAAAAAA | 38 | 8 |
| | CCCCCCCC | | |
| dA30C4 | AAAAAAAAAAAAAAAAAAAAAAAAAAAAAA | 34 | 9 |
| | CCCC | | |
| dA30C4-v2′ | AAAAAAAAAAAAAAAAAAAAAAAAAAAAAA | 54 | 10 |
| | CCCCGTCGCTCTCTCAAGTAGAAT | | |
| Control Random | GATGAGGTACCCAGAAGCCGAACTGGGC | 47 | 11 |
| | CAGACAACCCGGCGCTAAC | | |
| Control Random | AAAAAAAAAAAAAAAGATGAGGTACCCAGA | 76 | 12 |
| flanked | AGCCGAACTGGGCCAGACAACCCGGCGCT | | |
| | AACAAAAAAAAAAAAAA | | |
| DNA-Luc ASO | ATATCCTTGTCGTATCCC | 18 | 13 |
| DNA-Scramble | AATAATTGAATTCTTCnAA | 19 | 14 |
| ASO | | | |
| Survivin sense | AAAAAAAAAAAAAAAAAAAAAAAAAAAAAA | 46 | 15 |
| gel handle | TTACAAGCAGGTTCCT | | |
| Scramble sense | AAAAAAAAAAAAAAAAAAAAAAAAAAAAAA | 49 | 16 |
| gel handle | TTACTTGAAGAATTCAATT | | |

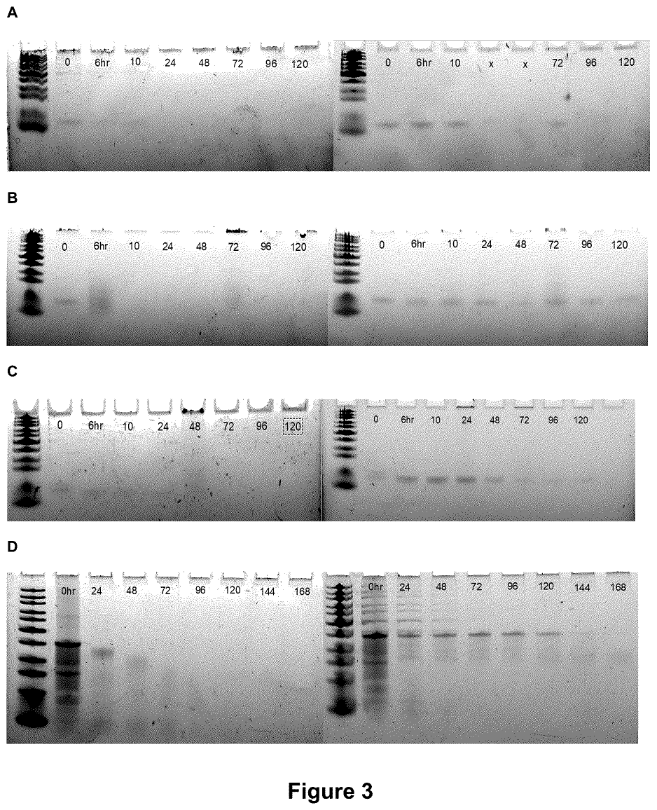

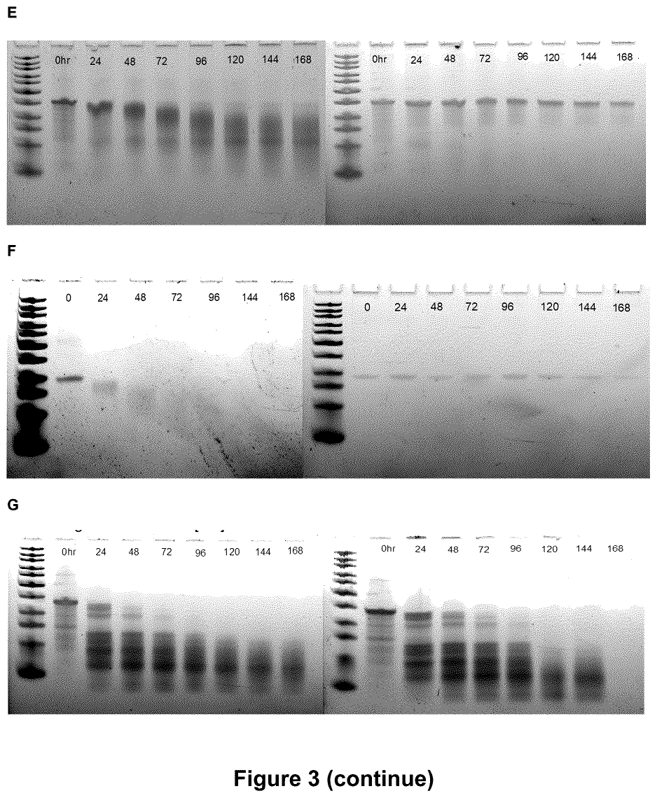

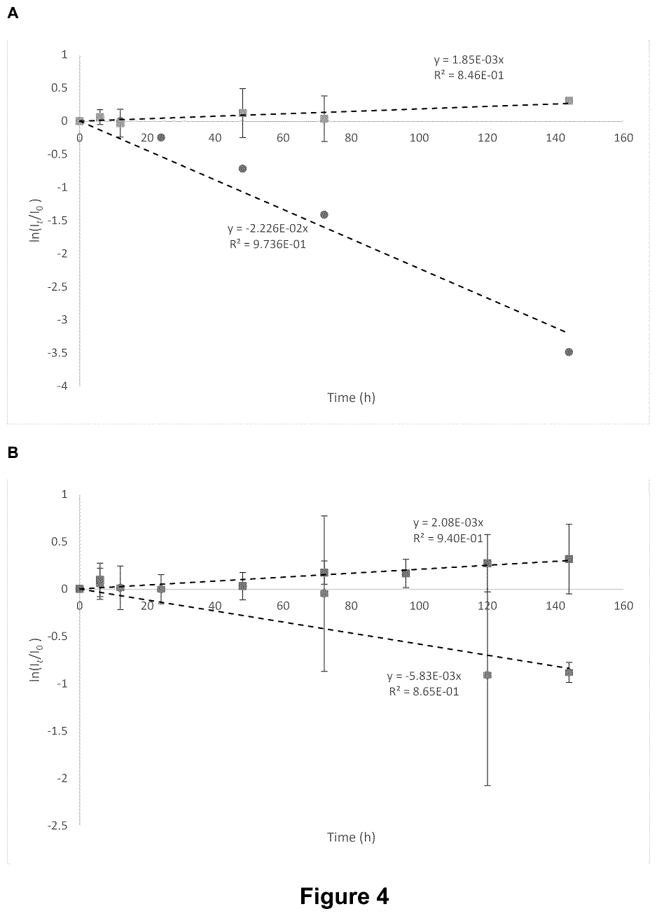

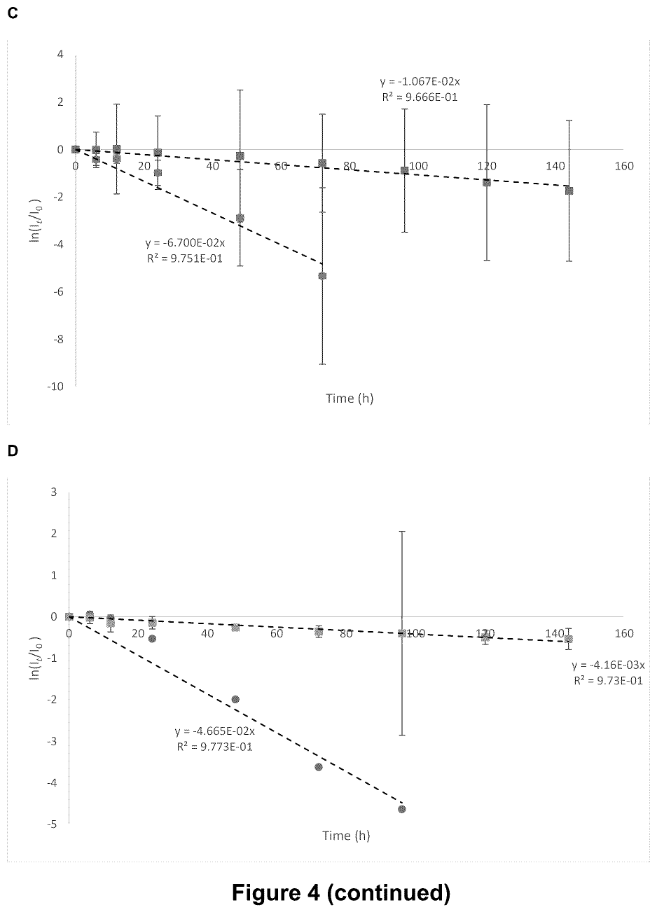

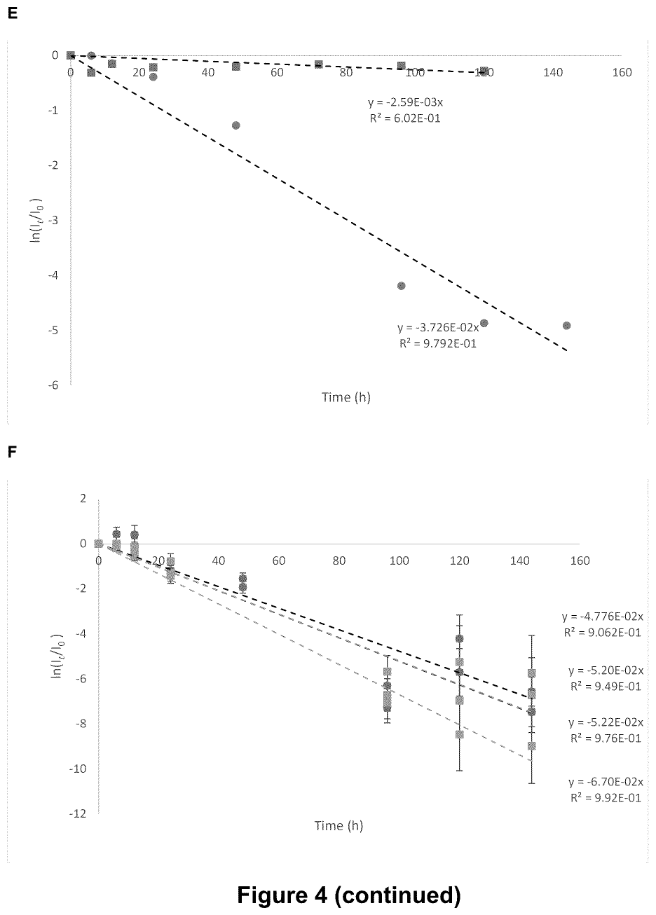

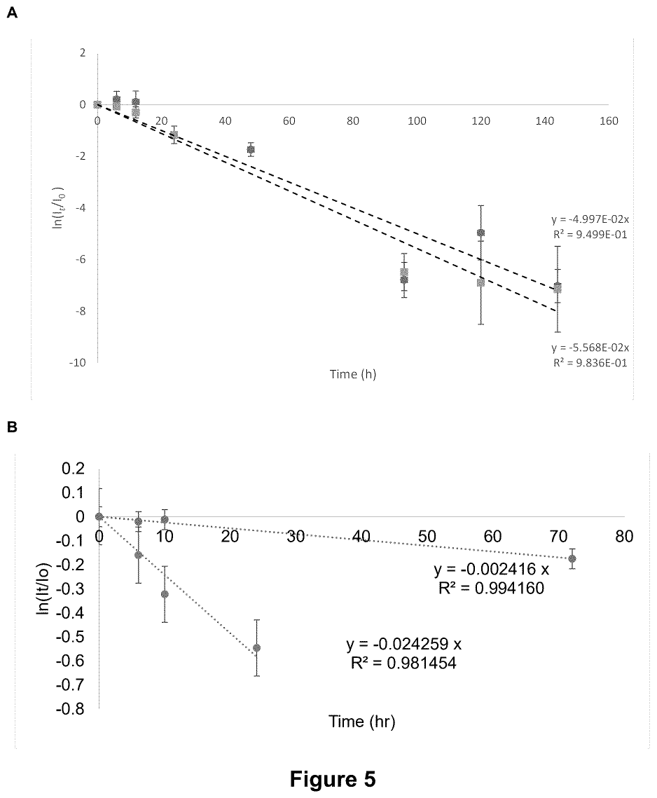

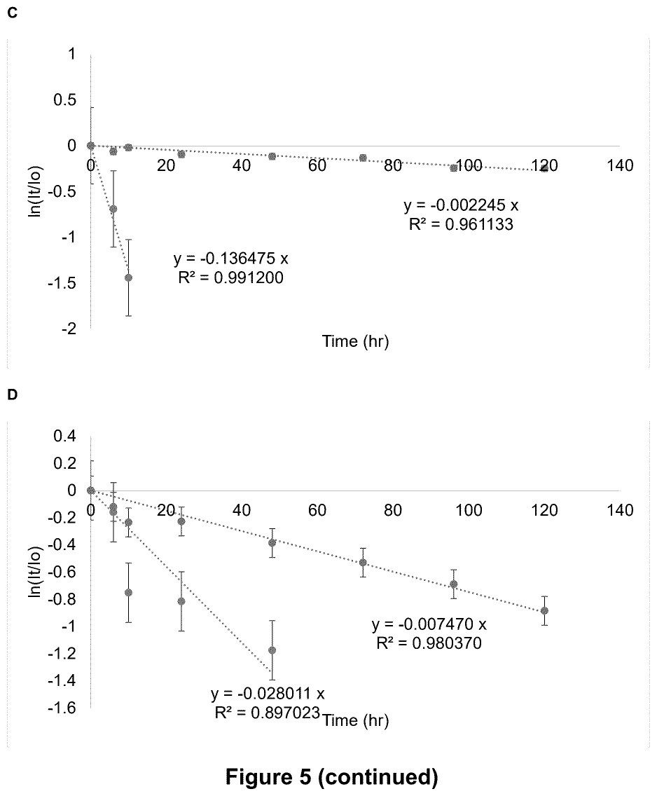

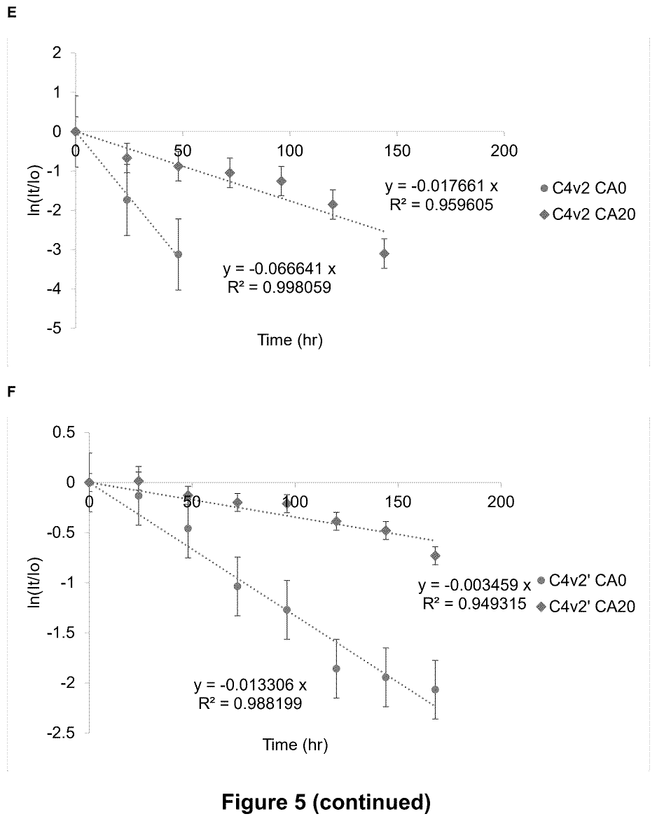

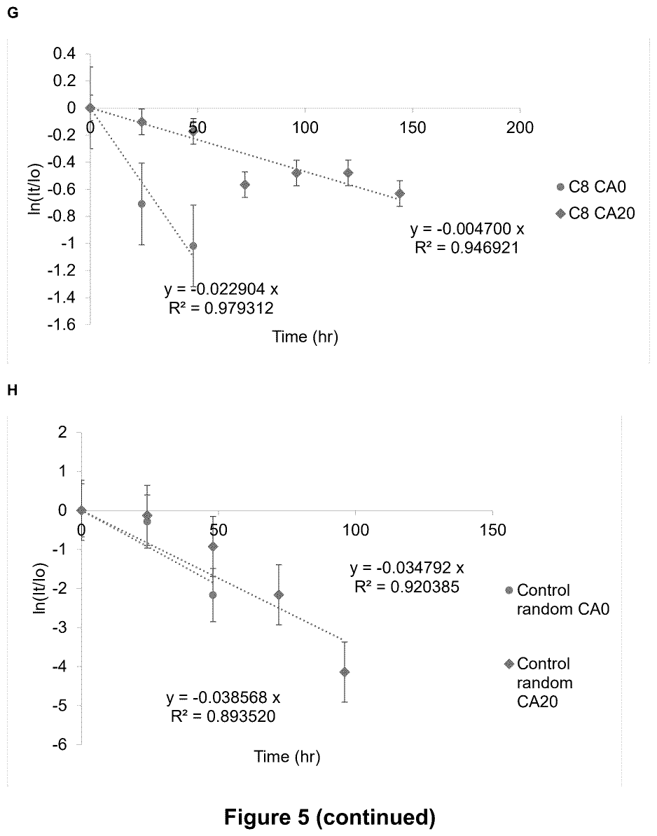

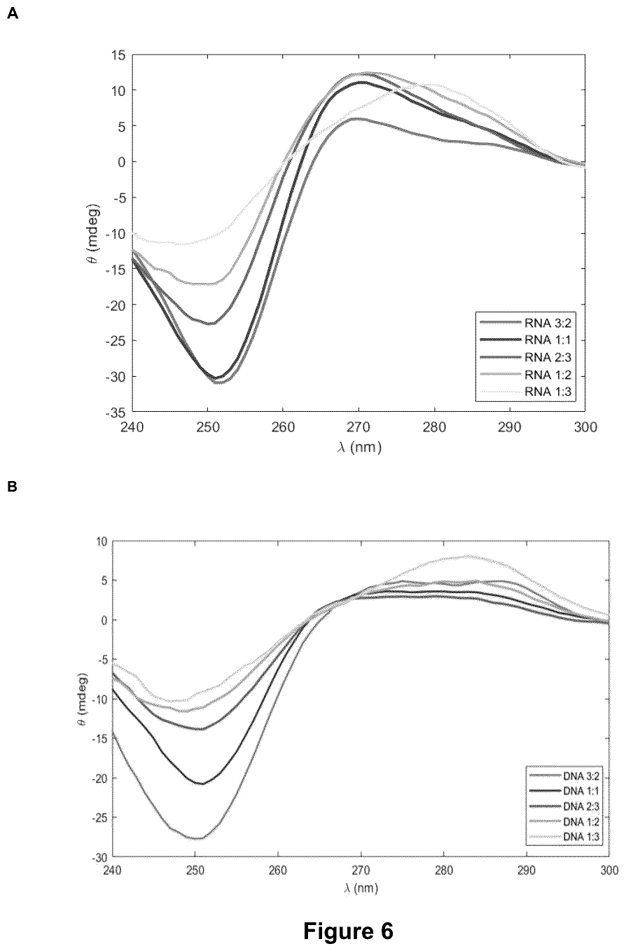

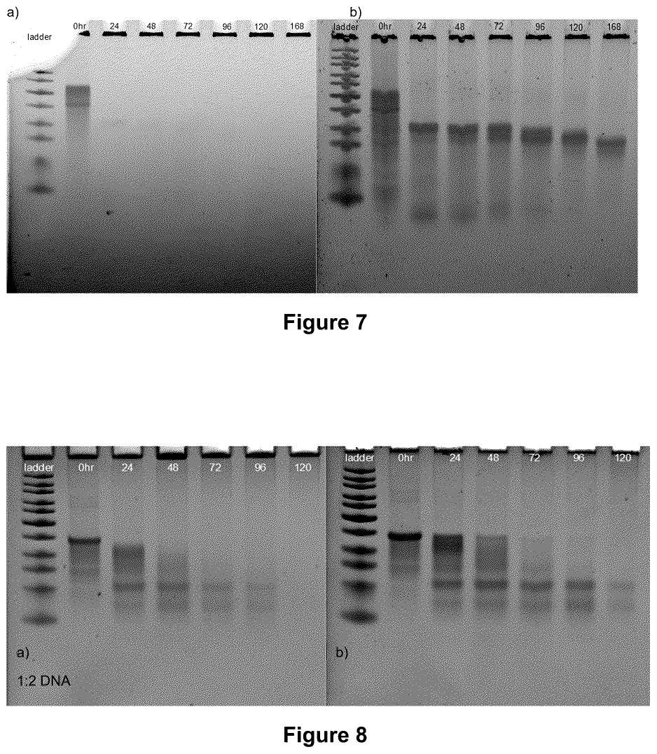

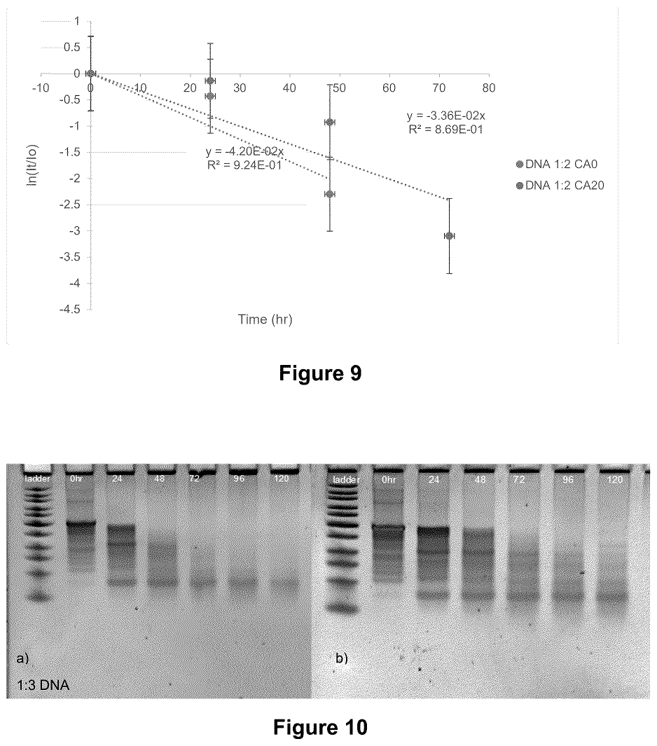

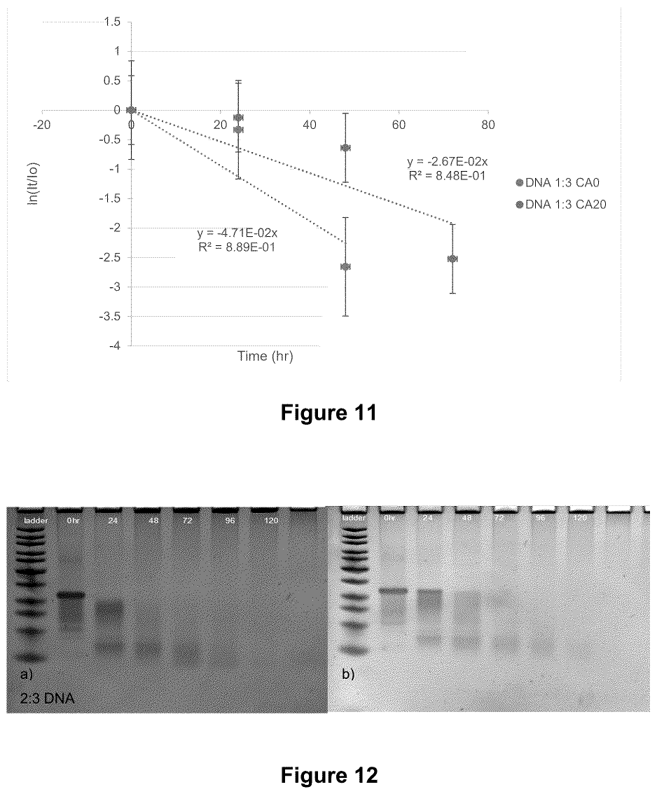

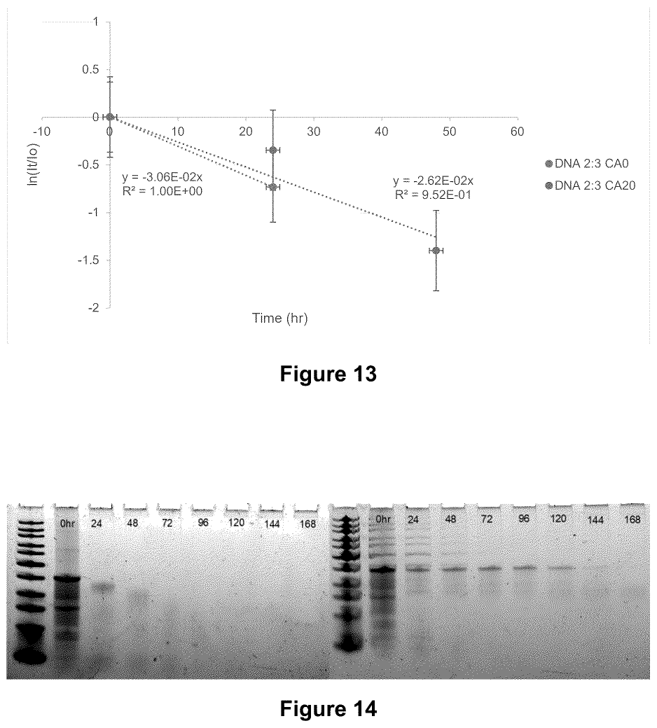

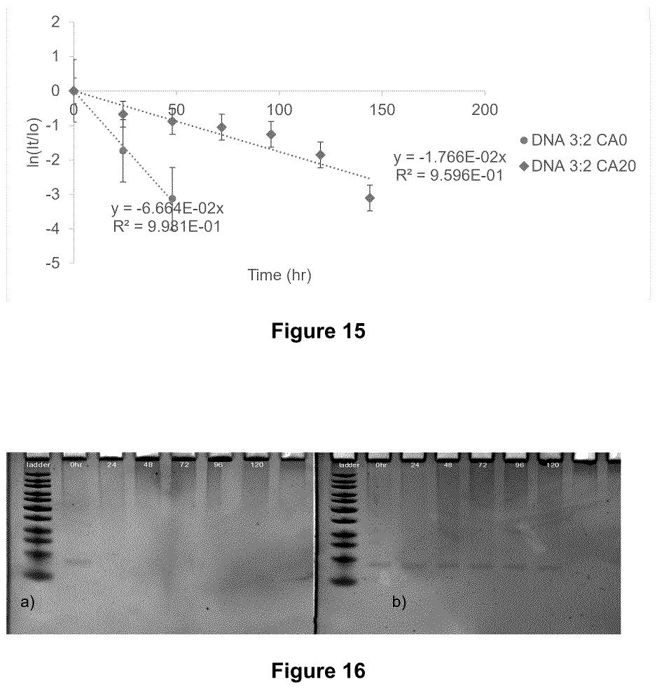

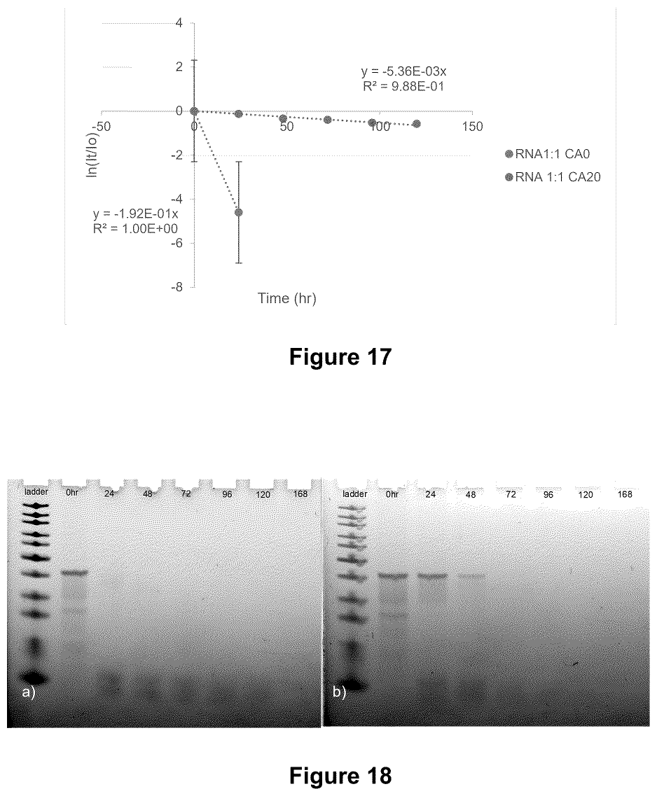

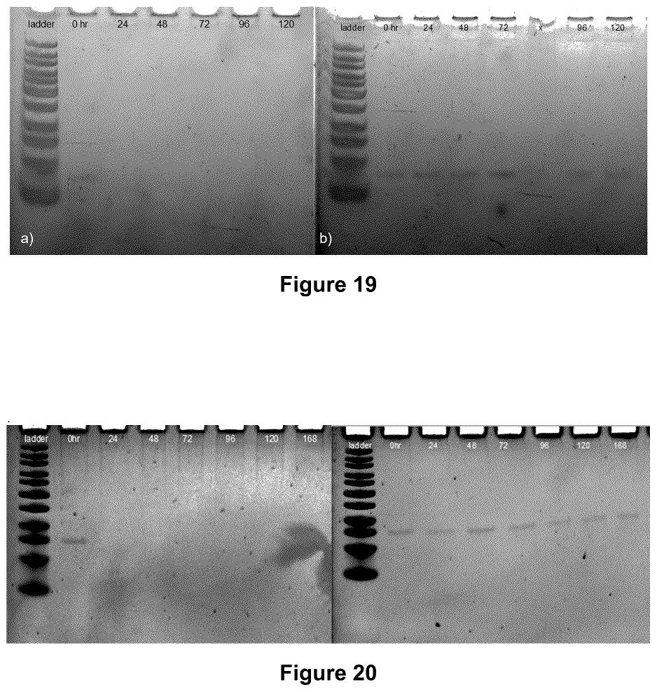

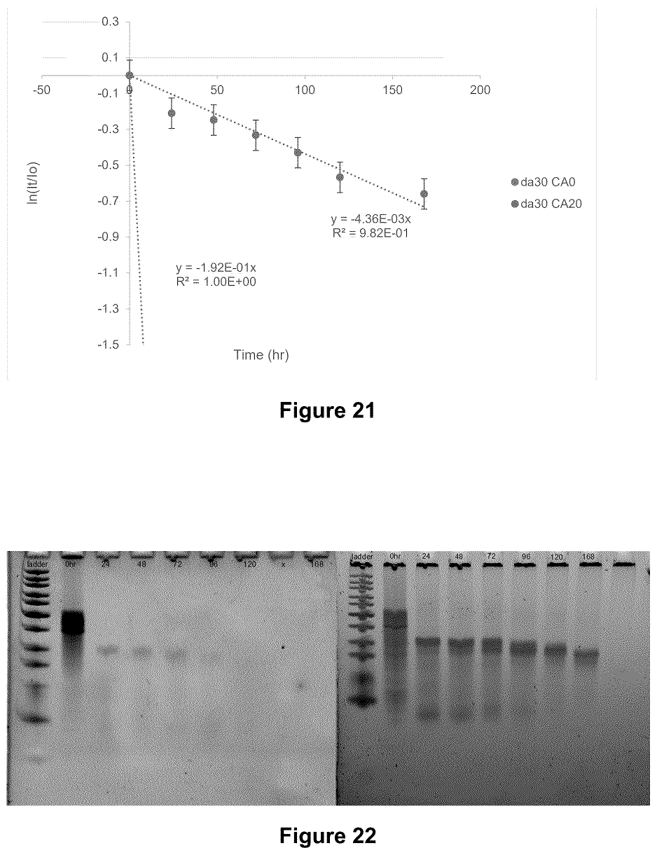

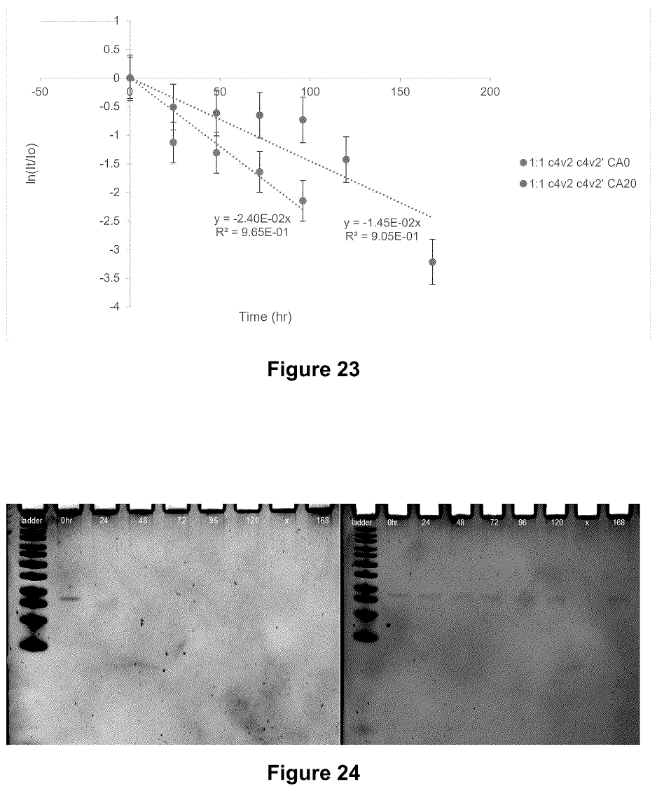

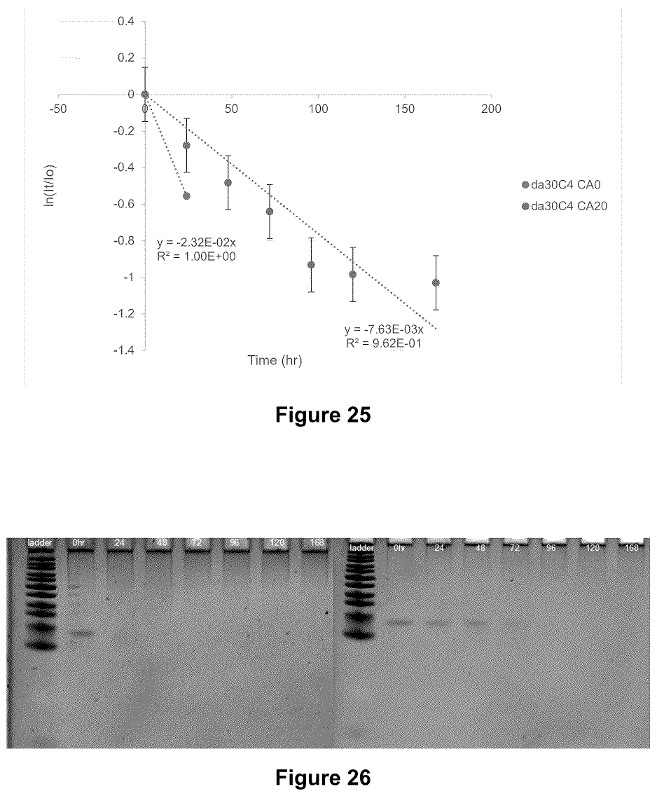

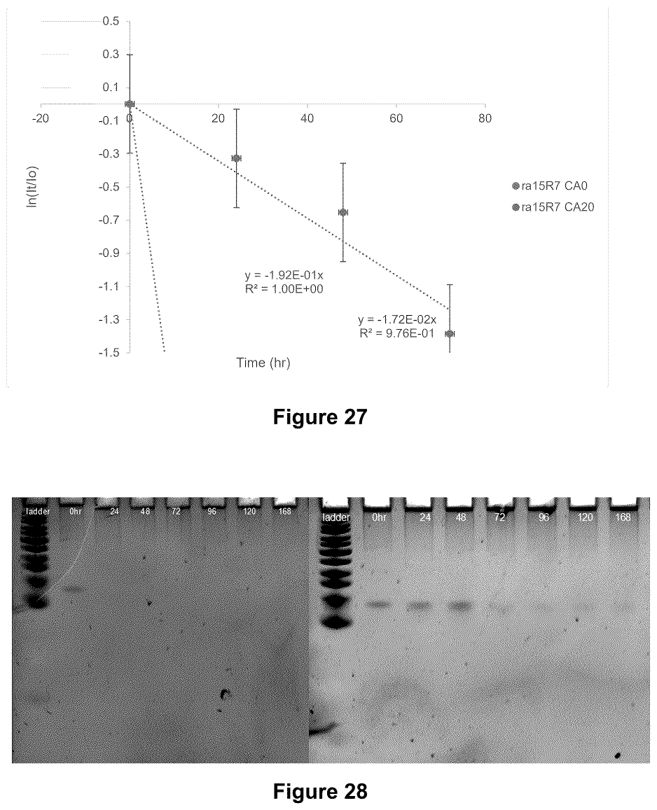

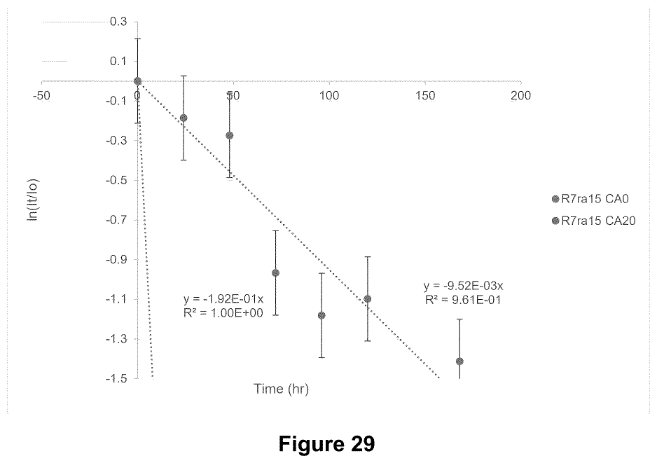

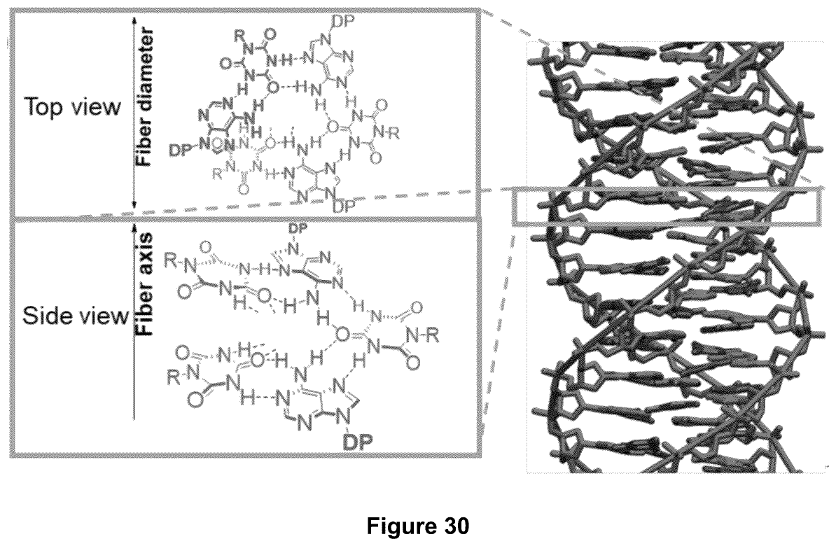

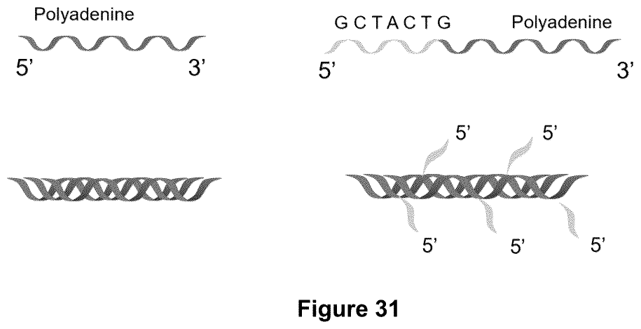

Results. The PAGE analyses were performed for both CA concentrations, and imaged side-by-side (FIG. 2). The strongest resulting band intensities were normalised to the t=0 data-point, plotted as a function of time (See Supplemental), and then fitted to the first-order kinetics equations (Equations 1, 2, and FIG. 3). The rate constants were calculated from these graphs (using Equation 4), and tabulated according to strand and CA concentration (Table 2).

$${[ NA ]}_{t} = {[ NA ]}_{0} {e}^{- k t} \qquad ( 1 )$$

Equation 1. First-order rate law for the variation of the concentration of a substance (in this case, nucleic acid, “NA”) with time, where [NA]ₜ is the concentration of NA at a given time t, and [NA]₀ is the initial concentration.

$$∴ ln ( \frac{{[ N A ]}_{t}}{{[ N A ]}_{0}} ) = - k t \qquad ( 2 )$$

Equation 2. The Equation 1, solved for kt, to depict the relationship between rate constant k and the concentration of NA as a function of time

$${ln [ NA ]}_{t} = - k t + {ln [ NA ]}_{0} \qquad ( 3 )$$

Equation 3. The Equation 2, rewritten in slope-intercept form, highlighting how the graphical fit reveals the rate constant, as the slope of the plot of In([NA]) vs. t

$$ln ( \frac{{I}_{t}}{{I}_{0}} ) = - k t \qquad ( 4 )$$

Equation 4. The Equation 2, written as relationship between the band intensities (I at some t, and at t=0), and the rate constant, k.

TABLE 2
Rate constants for strands 1-6 in L15 media (15% FBS, pH 6).
Values with * are negative probably due to uncertainty.

| Strand | k 0 mM [CA] (h⁻¹) | k 20 mM [CA] (h⁻¹) |
|---|---|---|
| dA30 | (2.22) × 10⁻² | (−1.85) × 10⁻³ * |
| dA30C4 | (5.83) × 10⁻³ | (−2.08) × 10⁻³ * |
| rA15 | (6.70) × 10⁻² | (1.07) × 10⁻² |
| rA15R7 | (4.67) × 10⁻² | (4.16) × 10⁻³ |
| R7rA15 | (3.73) × 10⁻² | (2.59) × 10⁻³ |
| Control | (5.00) × 10⁻² | (5.57) × 10⁻² |

TABLE 3
The rate constant of degradation of the set
of strands in Serum media (15% FBS, pH 6)

| Strand | k 0 mM [CA] (h⁻¹) | k 20 mM [CA] (h⁻¹) |
|---|---|---|
| rA15 | (2.43 ± 0.3) × 10⁻² | (2.42 ± 0.2) × 10⁻³ |
| rA15R7 | (1.25 ± 0.2) × 10⁻¹ | (2.25 ± 0.2) × 10⁻³ |
| R7rA15 | (2.80 ± 0.7) × 10⁻² | (7.47 ± 0.4) × 10⁻³ |
| dA30C4-v2 | (6.66 ± 0.4) × 10⁻² | (1.77 ± 0.3) × 10⁻² |
| dA30C4-v2′ | (1.33 ± 0.1) × 10⁻² | (3.46 ± 0.5) × 10⁻³ |
| dA30C8 | (2.29 ± 0.5) × 10⁻² | (4.87 ± 0.7) × 10⁻³ |
| Control random | (3.86 ± 0.2) × 10⁻² | (3.48 ± 0.8) × 10⁻² |

Discussion. The strands were chosen as to represent a variety of protection configurations. The pure polyA in DNA and RNA were noticed to have exceptionally long lifetimes, therefore an attempt was made to transfer the protection of these regions to flanking regions, themselves not forming the dA/CA assembly, but being in close proximity of it. Due to the differences in their rates and concentration, these flanking regions were added to either side of the polyA regions, as well as in the middle of a polyA region themselves. A dsDNA region and an internal hairpin were also added to verify their stabilities.

The rate constants, for rate of degradation, were less-negative in the CA 20 samples. In cases where the rate constant is positive, this has no physical meaning, but may be interpreted as a rate of 0 s⁻¹. It seems in all cases, the samples containing CA were more stable (less-negative rate constants, therefore slower rates of degradation), with minimal degradation after the final time-point (t=6 days). By comparison, 62 hours was the longest half-life achieved by a DNA sample, that could be found in literary sources, and this was obtained using a complex, 3-dimensional DNA origami structure, with chemically-modified ends.⁵ There was seemingly no correlation between strand length and rate of degradation, at least for the strands selected, although the rates of degradation were slower for DNA than RNA. Literary sources have shown that similar DNA sequences, including those assembled into 3D structures, tend to have half-lives on the hourly scale, which prompts further questions into the mechanism by which CA stabilises these strands.⁵. Specifically, by showing similar degrees of stabilisation in both the rA15R7 and R7rA 15 species, it may be assumed that both 5′->3′ and 3′->5′ directionally-specific exonuclease activity (such as exonuclease T, and RecJf) is impacted, as well as RNA-specific nucleases.^{4, 8}

There are several explanations for these remarkable results, including deactivation of exo- and endonucleases by the CA, and a lack of exo- or endonucleases present in the serum containing CA20. To further resolve the understanding of these results, the control experiment was performed, in triplicate, using the same process—and, notably, the same serum stocks—as the previous samples.

Given the results of the control study, which did not contain polyA regions in its sequence, but which, was a standard sample of DNA, it would seem that the interaction between CA and polyA regions of NAs (resulting in networked, fibrous structures) confers a degree of stability to NA strands, the likes of which have not been recorded previously.

Sources of error during experimentation include inconsistency during the aliquoting of samples and gel loading, as well as discrepancy in staining due to differences in exact staining times, or variation in stain solution concentration. Despite these sources of deviation, the presence of 20 mM CA imparts considerable serum stability on both the DNA and RNA strands tested. Repeat measurements, including on a wider variety of NA sequences, particularly NAs with varying overhangs, will assist in better establishing to what degree the presence of CA improves the stability of these nucleic acids, and where there are any exceptional cases in this observation, such as in the case of the control, where CA does not form the stabilising NA structures. Studies are being conducted, employing mRNA as the test strand, which naturally contains polyA tail regions. In one option, CA forms fibres out of the mRNA thereby stabilising these strands as well.

This information is also useful for preparation, transportation, and long-term storage of NAs, by including CA in samples, to stabilise them under biological conditions. This is particularly relevant to NA therapeutics, many of which currently require drastic measures (such as external coolants) to maintain stability.¹

### Example 1B—Degradation Experiments with Specific Enzymes

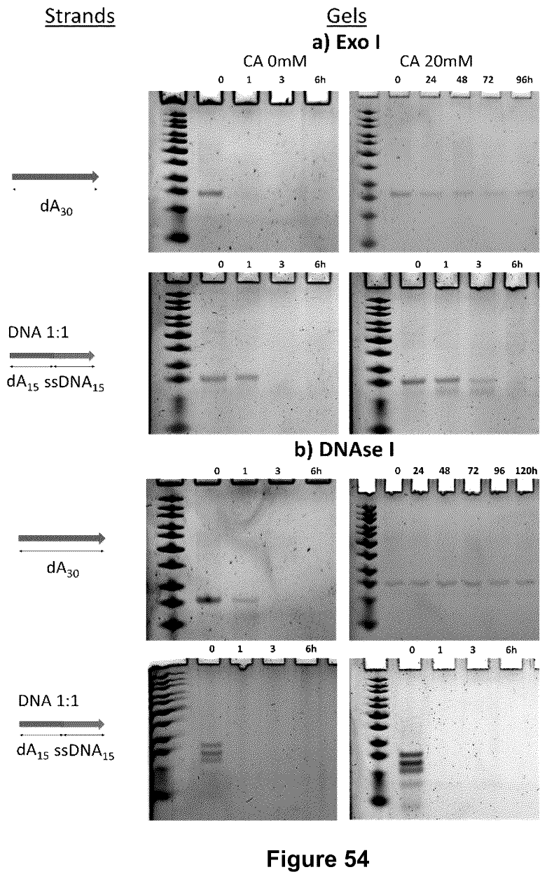

The fetal bovine serum used to test the biological stability of strands in presence of CA contains a mixture of multiple nucleases, including both endo- and exonucleases. The contribution of each kind of nuclease to degradation is unclear. The sequence of dA₃₀ and DNA 1:1 were submitted to degradation with both the endonuclease DNAse I and the exonuclease EXO I at the arbitrary concentration of 1 unit (FIG. 54).

Mirroring its long stability in fetal bovine serum, the dA₃₀ strand in the presence of CA becomes very stable to both Exo I and DNAse I, having the timescale of its degradation passing from minutes to days. In the case of dA₃₀ and DNAse I, even 5 days of degradation at 1U of enzyme does not cause degradation while the half-life without is only 27±6 min. It seems it is very difficult for the DNAse enzyme to cleave pure polyA/CA motif. Degradation by Exo I is more successful, albeit also significantly slowed, with a half life of t_{1/2}=63±15 h in the presence of CA versus less than 1 h without. Consequently, while the polyA/CA motif is harder to digest than polyA itself, Exo I can still slowly degrade it.

Like in fetal bovine serum, the DNA 1:1 strand in presence of CA is not as long-lived as the dA₃₀ strand. The addition of CA does increase its half-life slightly from t_{1/2}=30±10 min to 1.2±0.4 h in the presence of Exo I. The pattern of degradation observed for Exo I and DNA 1:1 with CA is somewhat unusual in that a sub-band is produced rather than a smearing characteristic of exonuclease nucleotide by nucleotide degradation. This seems to indicate the enzymes removes nucleotides until a certain length, then slows down for the rest of the sequence.

DNAse however cleaves the strand immediately in the timeframe required for pipetting the sample, with or without CA. Having a hold of the overhang allows the enzymes to digest the entire sequence, not leaving the polyA tail behind. These experiments were not done with the additional dA₃₀ filler addition which could further ameliorate the half-lives.

### Example 2-Optimizing Polyadenine Strands

The PAGE gels collected have time 0 hr set as reference strands (I₀), which is standardized as 1 where the intensity of other time point strands (It) is based on of. The intensity of each strand is standardized by

$$ln ( \frac{{I}_{t}}{{I}_{0}} )$$

and plotted against time in hours by the first-order rate law. The intensity of each strand is proportional to the concentration of the strand; therefore, the quantification of degradation would use the relative intensity of each strand. The band intensity collected is the average of the triplicate PAGE gels. The band quantified is the highest band, which is the fragment with the highest molecular weight among all denatured strand fragments as the calculation of the rate constant is for degradation.

TABLE 4
The sequence of the RNA and DNA strands varying in the ratio between the adenine
sequence and the non- adenine sequence. The length of the polyadenine sequence is
constant with 15 bases. In the ratio, the first number is for the polyA
and the second number is for the non A part.

| Sequence | | SEQ ID NO |
|---|---|---|
| RNA 1:3 | rArArA rArArA rArArA rArArA rArArA rUrCrA rCrCrA rCrGrU | 17 |
| | rArArA rCrArU rGrGrG rUrCrU rUrArG rUrUrG rArCrU rGrUrA | |
| | rCrCrA rUrCrU rGrCrA rArCrG | |
| RNA 1:2 | rArArA rArArA rArArA rArArA rArArA rUrCrA rCrCrA rCrGrU | 18 |
| | rArArA rCrArU rGrGrG rUrCrU rUrArG rUrUrG rArCrU | |
| RNA 2:3 | rArArA rArArA rArArA rArArA rArArA rUrCrA rCrCrA rCrGrU | 19 |
| | rArArA rCrArU rGrGrG rUrCrU rUrArG | |
| RNA 1:1 | rArArA rArArA rArArA rArArA rArArA rUrCrA rCrCrA rCrGrU | 20 |
| | rArArA rCrArU | |
| DNA 1:1 | AAA AAA AAA AAA AAA TCA CCA CGT AAA CAT | 21 |
| DNA 1:3 | AAA AAA AAA AAA AAA TCA CCA CGT AAA CAT GGG TCT | 22 |
| | TAG TTG ACT GTA CCA TCT GCA ACG | |
| DNA 1:2 | AAA AAA AAA AAA AAA TCA CCA CGT AAA CAT GGG TCT | 23 |
| | TAG TTG ACT | |
| DNA 2:3 | AAA AAA AAA AAA AAA TCA CCA CGT AAA CAT GGG TCT | 24 |
| | TAG | |

TABLE 5
The strands sequence used in mouse serum stability test.

| | | | SEQ ID |
|---|---|---|---|
| Sequence | | Length | NO |
| RNA polyA | AAAAAAAAAAAAAAA | 15 | 1 |
| (rA15) | | | |
| RNA polyA 3′ | GCTACTGAAAAAAAAAAAAAAA | 22 | 2 |
| nonA end | | | |
| (R7rA15) | | | |
| RNA polyA with | AAAAAAAAAAAAAAAGTCATCG | 22 | 3 |
| 5′ nonA end | | | |
| (rA15R7) | | | |
| DNA polyA | AAAAAAAAAAAAAAA AAAAAAAAAAAAAAA | 30 | 6 |
| (dA30) | | | |
| DNA poly A and | AAAAAAAAAAAAAAAAAAAAAAAAAAAAAACCCCATTCTACTTGAGAG | 54 | 25 |
| non A tail | AGCGAC | | |
| (dA30C4v2) | | | |
| DNA with I- | AAAAAAAAAAAAAAAAAAAAAAAAAAAAAACCCC | 34 | 26 |
| motif (dA30C4) | | | |
| DNA poly A and | AAAAAAAAAAAAAAAAAAAAAAAAAAAAAACCCCGTCGCTCTCTCAAG | 54 | 27 |
| complementary | TAGAAT | | |
| non A tail | | | |
| (dA30C4v2′) | | | |

As the concentration of reactant is the variable, the first-order rate law is used. To quantify the concentration of strand fragments, the relative intensity of each band would be representative of their concentration, in which concentration ([A]) is replaced by the intensity (I) in the equation.

$${[ A ]}_{t} = {[ A ]}_{0} {e}^{- k t} \qquad ( 5 )$$

$${I}_{t} = {I}_{0} {e}^{- k t} \qquad ( 6 )$$

$$ln {I}_{t} = - kt + ln {I}_{0} ln \frac{{I}_{t}}{{I}_{0}} = - kt \qquad ( 7 )$$

$$half life = \frac{In ( 2 )}{k} = \frac{In ( 2 ) \times t}{- ln \frac{{I}_{t}}{{I}_{0}}} \qquad ( 8 )$$

The graph plotted has the rate constant k as the slope, which helps to distinguish the effectiveness of CA in preventing the degradation of strands.

RNA and DNA strands varying in different A and non-A ratio. DNA and RNA strands with ratio varying from 3:2, 1:1, 1:2, 2:3 and 1:3 were tested. From the CD spectrum of both DNA and RNA strands, the higher the proportion of the polyadenine stretch, the more intense the peak of the CA/polyA assembly at 252 nm (see FIG. 6, where the depth of the 252 nm peak is indicative of the amount of polyA/CA structure formed). With each of these strands varying with length from 22 to 60 bases, there should not be significant second order structures as the non-A sequences were designed to avoid higher order structure and these length range is the range that observed to have significant protection. The CD spectrums suggests that non-A sequence can interfere the contact between the CA and polyA, which forms either shorter CA/polyA fiber or fewer longer CA/polyA fiber. The CD data can be further supported by the gel images and degradation plot of the ratio strands. Quantitatively observe the gel images, the top band in FIGS. 8 (b), 10 (b) and 12 (b) for DNA 1:2, 1:3 and 2:3 strands, they are preserved for one more day comparing to the untreated ones. Whereas the 3:2 strands in FIG. 14 can preserve up to 7 days. 3:2 ratio one has significant signal in CD, indicating it has a longer fibrous assembly that establish a greater steric hinderance for nucleases. While DNA strands with more non-A sequence still has CD dips observed, the formation of shorter hexametric assembly fails to protect non-A sequence from both endo and exonucleases. This is demonstrated by the bands at around 15 bp and some 30 bp in FIGS. 8, 10 and 12. This can suggest that the longer the non-A sequence the more interference that CA encounters in forming the assembly as the CD dip at 252 nm is also lowered with the increase in non-A sequence. If the CD signal among each strand with different ratios are similar, then the nuclease resistance is independent of the length and size of the CA/polyA fibrous assembly. From this experiment, it would be interesting to push the formation of the fiber by using different CA derivatives that can push by the concentration of the CA-derivative in the buffer and the formation of CA/polyA fiber. So we can see of the CA/polyA fiber's capacity of protecting the non-A sequences. This experiment again supports the point that the longer the non A sequence, the more the polyA sequence would be hidden from CA that results in a less effective assembly.

TABLE 6A
The degradation rate constant of the ratio
strands in CA0 and CA20 conditions.

| | k CA0 (hr − 1) | k CA20 (hr − 1) | k CA0/k CA20 |
|---|---|---|---|
| 1:2 DNA | 0.042 | 0.0336 | 1.25 |
| 2:3 DNA | 0.047 | 0.0267 | 1.7603 |
| 1:3 DNA | 0.0306 | 0.0262 | 1.167939 |
| 3:2 DNA | 0.06664 | 0.0176 | 3.786364 |
| 1:1 RNA | 0.192 | 0.0536 | 3.58209 |

TABLE 6B
Half-lives of the ratiometric sequences, with or without CA
and with or without added dA₃₀. There is a relevant difference
in half-life between the two conditions if p > 0.95.

| | CA 0 mM | | CA 20 mM | | Ratio | | |
|---|---|---|---|---|---|---|---|
| Sequence | t_{1/2} | (h) | t_{1/2} | (h) | CA20/CA0 | | p-value |
| DNA 1:1 | 13 | ±3 | 30 | ±4 | 2.3 | ±0.7 | 0.98 |
| DNA 2:3 | 13 | ±4 | 22 | ±3 | 1.7 | ±0.6 | 0.99 |
| DNA 1:2 | 19 | ±4 | 25 | ±6 | 1.3 | ±0.4 | 0.86 |
| DNA 1:3 | 20 | ±5 | 19 | ±3 | 1.0 | ±0.3 | 0.59 |
| DNA 2:3 long | 35 | ±4 | 40 | ±4 | 1.1 | ±0.2 | 0.87 |
| DNA 1:1 + dA30 | 7 | ±1 | 64 | ±12 | — | | 1.00 |
| DNA 2:3 + dA30 | 18 | ±2 | 47 | ±13 | 2.6 | ±0.8 | 0.97 |
| DNA 1:2 + dA30 | 16 | ±4 | 61 | ±18 | 3.8 | ±1.4 | 0.99 |
| DNA 1:3 + dA30 | 13 | ±2 | 30 | ±8 | 2.4 | ±0.7 | 0.99 |
| DNA 2:3 long + dA30 | 22 | ±2 | 67 | ±15 | 3.0 | ±0.7 | 1.00 |
| Control | 18 | ±3 | 16 | ±3 | 0.9 | ±0.2 | 0.77 |
| Control + dA30 | 17 | ±3 | 12 | ±3 | 0.7 | ±0.2 | 0.93 |

The CD indicates that there is a decreased ability to form the polyA/CA motif with shorter polyA relative to non-polyA sequences. The mechanism underlying such a loss in the motif could be due to the steric interference emanating from the protruding non-polyA sequences clashing with one another. The larger the non-polyA region compared to the polyA, the closer these sequences will be pushed together, creating an unfavourable environment for the formation of the fibers. This decreased fiber formation mirrors a decreased protection by the addition of cyanuric acid for the longer non-polyA sequences. For the shortest sequence of 1:1, cyanuric acid increases lifetime 2.3±0.7 fold, which falls to 1.7±0.6 for the polyA:DNA 2:3 strand (Table 6B). PolyA:DNA sequences 1:2 and 1:3 fail to show an increase in lifetime with cyanuric acid.

The correlation of the polyA/CA fiber presence by CD and the nuclease resistance in strands suggests the main determinant for the protection of the sequence would be the ratio of the polyA/non-polyA region rather than their absolute length. To test this, a “long” sequence was made with the ratio of 2:3, but with a polyA length of 30 instead of 15, and so a total length twice as long as the “short” 2:3 sequence, while keeping the same ratio of polyA to non-polyA (Table 6B). The half-life of this strand with CA is slightly longer for the “short” sequence, but it does not reach statistical significance (ratios of 1.7±0.6 for the “short” 2:3 and 1.1±0.2 for the “long” 2:3). Since the same ratio leads to similar stabilization even if the absolute length of the overhang differs, the important parameter for stabilization then is the ratio of polyA to non-polyA, which determines the extent of fiber formation. In principle, there would thus be no limit to the absolute length of the sequence being protected, as long as it is accompanied by comparatively long polyA tails. One method to increase the polyA to non-polyA ratio is to use polyadenylate polymerase to attach polydisperse but long polyA to sequences, allowing them to form polyA/CA fibers.

If the reduction in fiber formation results in a decrease in protection, and if this reduction is caused by steric clashes arising from protruding non-polyA sequences, then adjusting the spacing of these sequences could potentially restore nuclease resistance. We investigated whether the addition of “filler” polyA strands could act as spacers between these protruding non-polyA sequences. The free polyA could co-assemble with the other strands and would increase the distance between the overhangs, thereby allowing for less constraint and restoring fiber formation, and subsequently sequence protection. To do so, dA₃₀ sequences were added to the ratiometric strands in equimolar amounts and tested their nuclease resistance.

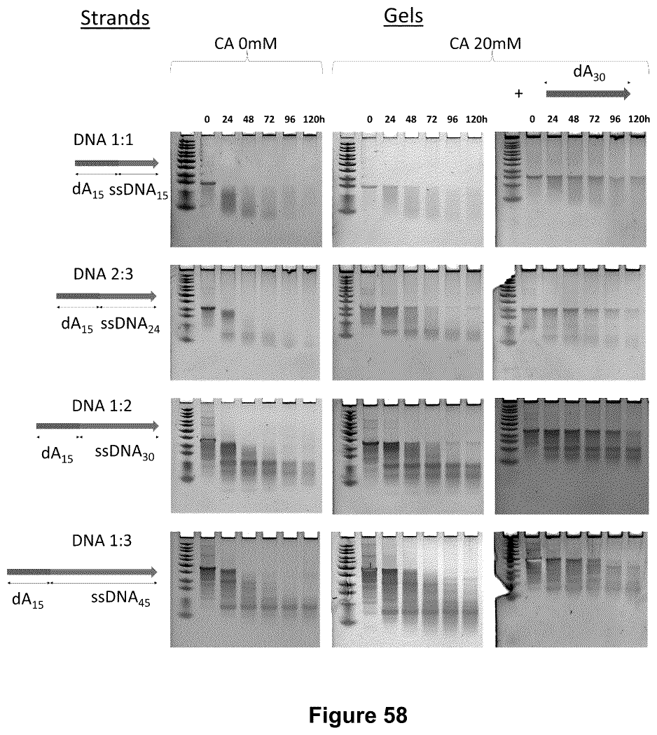

In line with the hypothesis, we could regenerate the protective effect of CA for all ratiometric sequences (FIG. 58, Table s). It is noted that this methodology increases the lifetime of the different ratiometric strands tested by similar amounts of 2.6±0.8, 3.8±1.4 and 2.4±0.7 times for polyA:DNA 2:3, 1:2 and 1:3 respectively (1:1 was excluded because it has the same length as the dA₃₀ filler sequence and thus cannot be separated by gel). In contrast, a control non-polyA sequence (without a polyA tail) in the presence of dA₃₀ did not lead to any significant stabilization with (t_{1/2}=12±3 h) or without CA (t_{1/2}=17±3 h), indicating that the polyA/CA is not a competitive inhibitor like actin²⁵ and that the incorporation into polyA/CA fibers is the determining factor in the stabilities observed.

The addition of filler strands significantly increases the sequence space which can be protected by the polyA/CA motif, allowing sequences of lengths of up to 60 bases at least to be protected for several days, in our conditions (FIG. 6, DNA 1:3). The rationale for this method was supramolecular, with the control on the polymeric fiber structure the key in increasing the half-lives. Seen through this lens, the supramolecular toolbox at our disposal to modulate the polyA/CA fibers extends beyond the polyA portion of the assembly. The cyanuric acid, the defining small-molecule feature which renders this form unique, is also a convenient handle to tune the assembly, and thus perhaps its resistance to nucleases.

Serum stability test in acidified-MES buffer 10% mouse serum. With the previous MES buffer containing 10% FBS, we have tried to use another media mouse serum as it has more endonucleases. The nuclease resistance significantly dropped in strands with longer non-A sequence. It implies that the protection that CA brings is weaker against endonucleases and tends to cleave around 14 BP away from the polyA region.

### Example 3-Protecting Oligonucleotides Using Hydrogels

Combining the processability of solids with the diffusion-enabled reactions of liquids, nucleic acid hydrogels-hydrogels formed by supramolecular interactions between nucleic acid strands-present numerous valuable characteristics. The biocompatibility and therapeutic uses of nucleic acids make their hydrogels well-suited for biological applications, such as drug delivery,^{[}2] vaccine formulations,^{[}3] gene-activated matrices^{[}4], cellular growth and tissue engineering.^{[5}] Beyond these classical applications, the sequence control of DNA hydrogels endows them with unique properties. From selective control of cargo diffusion,^{[}6] sequence-specific changes in moduli,^{[}7] chemical dynamic networks,^{[7b, 8]} and tunable sensing of mechanical forces,^{[}9] the programmable nature of nucleic acids results in the design of hydrogels with precise and complex stimuli-responsive behavior.

For all their advantages, however, DNA hydrogels have issues that prevent their wider applications. First of these is the low mechanical stiffness of unmodified DNA hydrogels, with reported storage modulus (G′) from 10¹ Pa to 10³ Pa, with the recent 4×10³ Pa at 3.5 wt % obtained by the Liu group considered high in the field. This low range of stiffness reduces their effectiveness in applications such as soft robotics, and precludes them from some stiffer cell environments such as the spleen.^{[11]} DNA hydrogels also suffer from the lack of a convenient chemical handle, preventing the incorporation of useful chemical modifications. Current methods to increase the accessible chemical space rely on costly and low-density synthetic modifications of the DNA strands.

An exemplary nucleic acid structure was developed from the interaction of polyadenine with the small non-toxic molecule cyanuric acid—the dA/CA motif (FIG. 1a).^{[13]} With three faces mimicking thymine, cyanuric acid acts as a bridge between adenines of three poly-dA strands (FIG. 1a), coaxing them into a supramolecular polymer. The polymer is a parallel DNA triple helix formed by a continuous cyanuric acid-adenine hydrogen-bonded helical structure.^{[14]} Already an unusual DNA secondary structure based on its incorporation of small molecules at high density, this motif also distinguishes itself by its spontaneous formation of multi-micron fibers which bundle together (FIG. 1c). It is hypothesized that these fibers could serve as a basis for stronger DNA hydrogels, closer to fibrous protein-based hydrogels, with a high density of functional groups stemming from the cyanuric acid and its ethylamine sidechain.

Using this dA/CA motif, a hydrogel built from unmodified DNA is reported which has a storage modulus of 10⁵ Pa at only 4.7 w %, which is two orders of magnitude higher than the stiffest reported DNA-based hydrogels, and which extends the modulus range of unmodified DNA hydrogels to 5 orders of magnitude. It can even conserve a very high modulus at 85° C. Key to navigating this space is the modularity of the system, in which either complementary overhangs of DNA or cyanuric acid sidechains can control the modulus. This hybrid DNA/small-molecule hydrogel also naturally exhibits sought-after properties such as high functional group density, thixotropy, fast self-healing and facile preparation. The hydrogel also showcases multi-stimuli responsiveness, including a small-molecule stimulus rare in DNA hydrogels, while conserving the stimuli responsiveness to specific DNA sequences. Provided herein are how these properties can be used to encapsulate and controllably release DNA wireframe and origami nanostructures. It will conclude by leveraging stimuli-responsiveness and thixotropy to make an injectable therapeutic hydrogel that enhances gene silencing activity in vitro.

### Results and Discussion

Tunability of mechanical properties. The hydrogels are formed readily by mixing the appropriate poly-dA containing strands with a solution containing cyanuric acid (CA) or its derivative (CAC2NH₂) at a slightly acidic pH of 6, then heating at 85° C. and cooling quickly to 20° C. for homogeneous, clear gel formation (FIG. 1e) (see Methods). With this simple assembly, extensive tuning of the resulting mechanical properties is possible by varying the nature of the components.

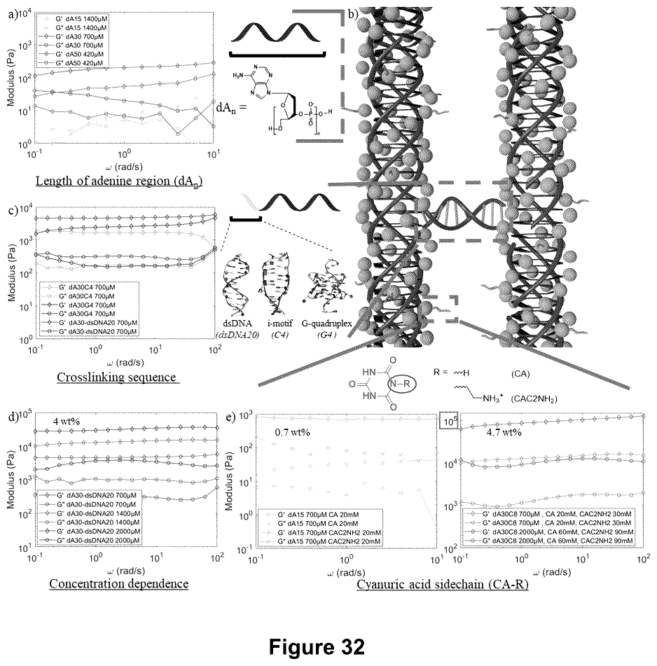

Polyadenine length. The first modular component in the design space of the hydrogel is the polyadenine region itself (FIG. 32, a), which binds to CA, resulting in the dA/CA fibers. The addition of CA to dA₁₅ gives rise to hydrogels, likely because of fiber entanglement^{[13, 15]} Changing the length of the dAₙ region affects the mechanical properties in a non-linear fashion. At the same weight % (wt %), the extension of the polyadenine strand from a 15-mer to a 30-mer strengthens the storage modulus (G′) from 1.3±0.8×10¹ Pa to 1.8±0.4×10² Pa at 1 Hz. This likely stems from the tendency of dA₃₀ to form more crosslinked networks of fibers compared to dA₁₅.^{[15]} Upon elongation to a dA₅₀ the storage modulus decreases to 4±2×10¹ Pa at 1 Hz, possibly a consequence of the strands being long enough to curl back and copolymerize intramolecularly, rather than with other strands. Gels with dA₃₀ strands were focused on in the present study because of their greater mechanical stiffness.

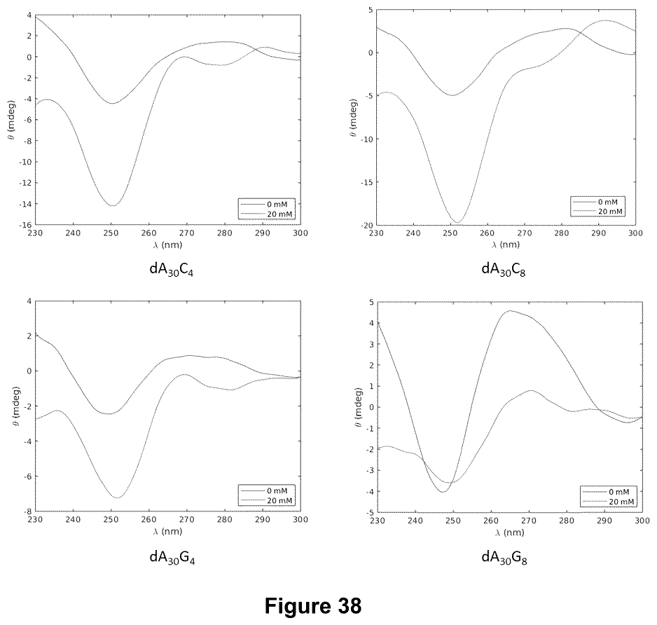

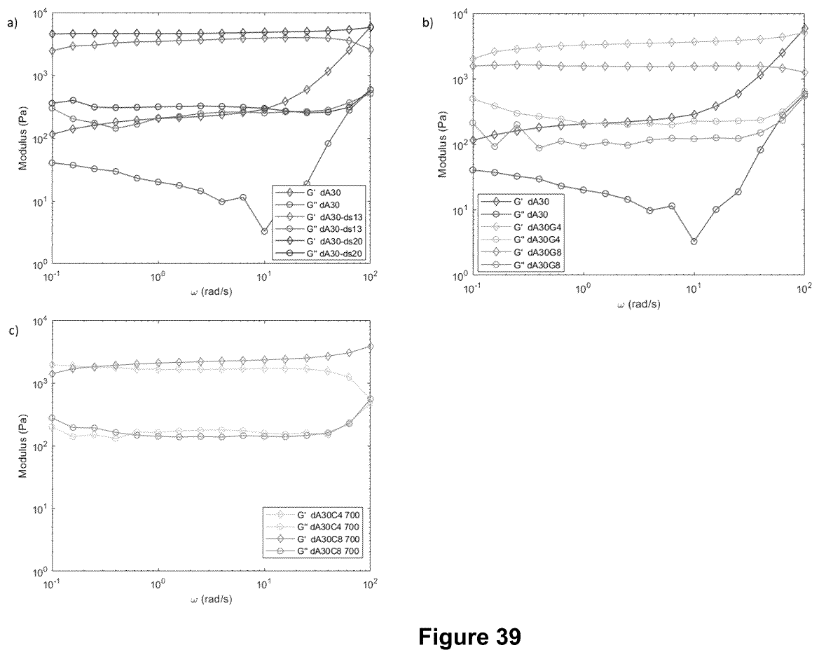

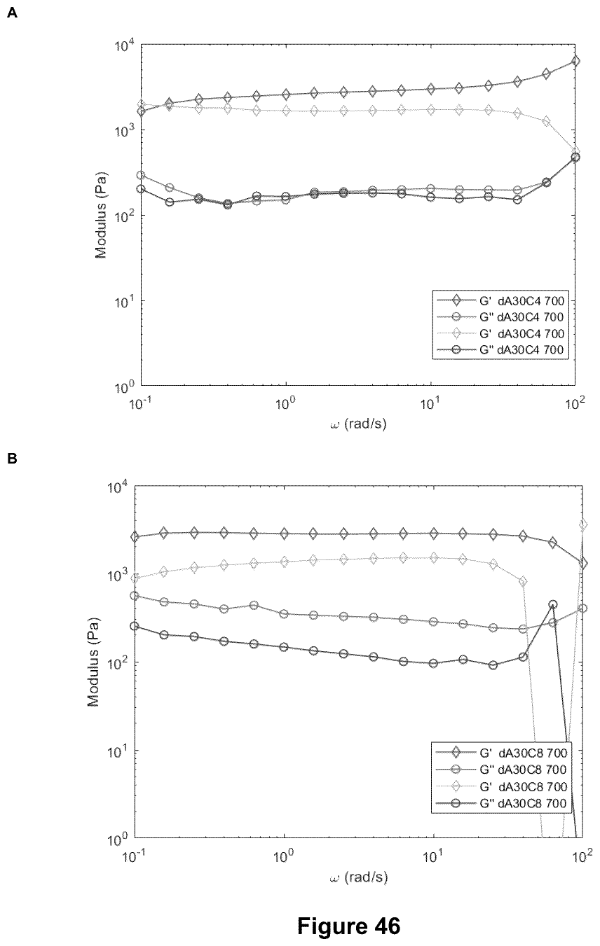

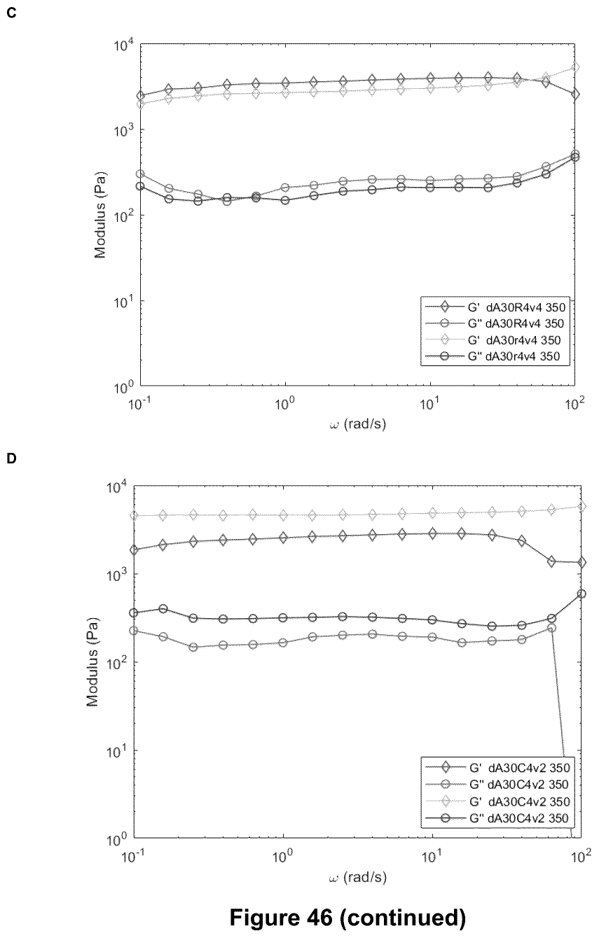

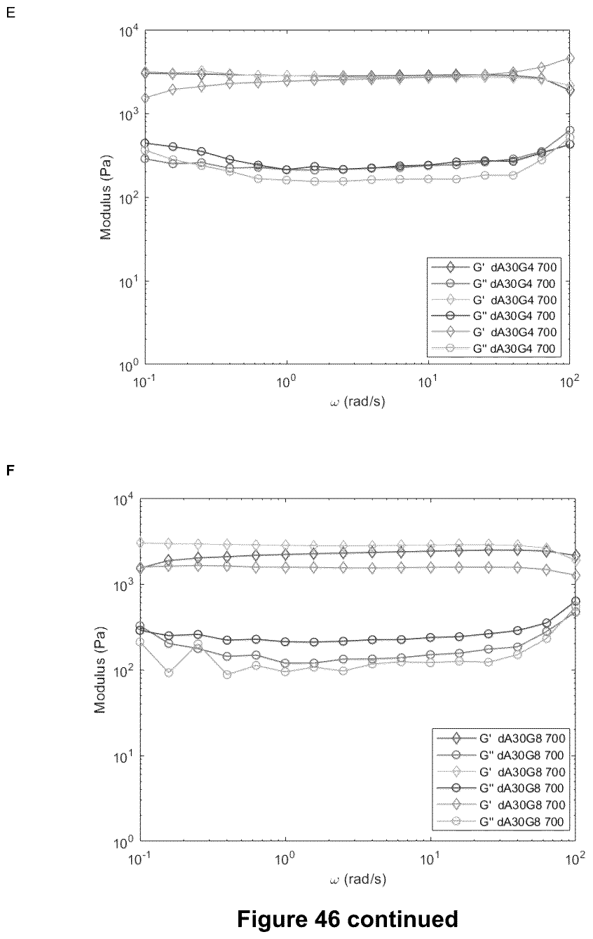

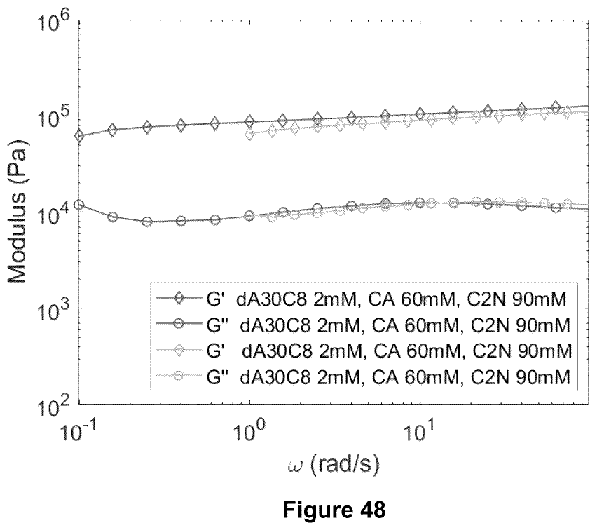

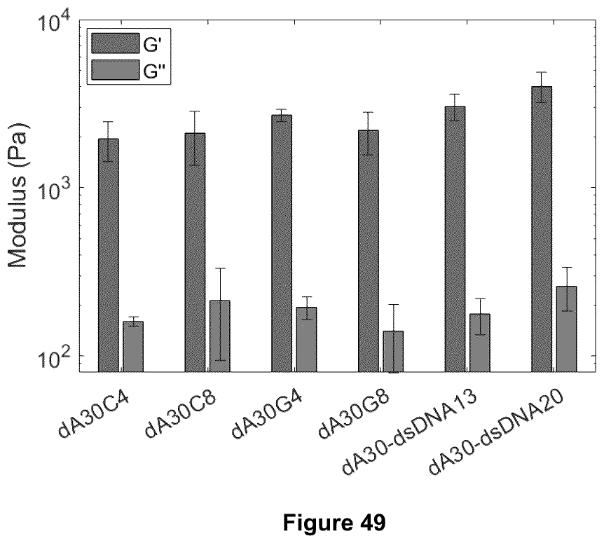

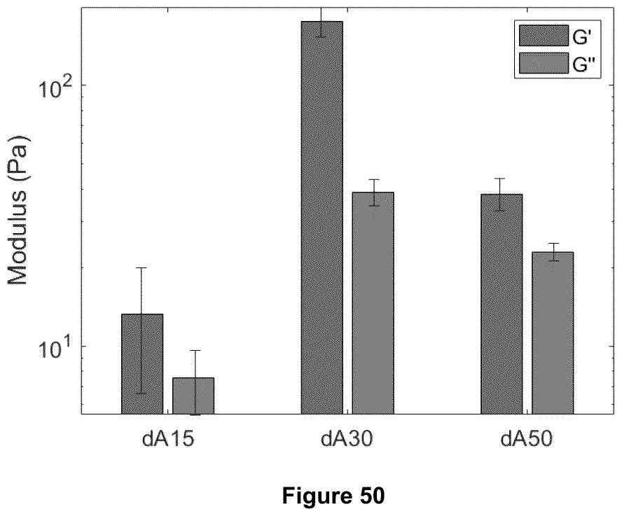

Crosslinking sequence. To introduce crosslinking between the dA/CA fibers and thus enhance the gel's mechanical properties, an overhang region at the end of the poly-dA strands was added. Overhang sequences that can interact with each other through DNA-based interactions mediate polyA/CA fibre crosslinking. These crosslinks improve the moduli of hydrogels versus uncrosslinked ones by an order of magnitude. For example, strands consisting of a stretch of 30 adenine followed by 20 complementary base pairs as an overhang (dA₃₀C₄v2) form double-stranded DNA crosslinks which increase the storage modulus G′ (1 Hz) to 4±0.8×10³ Pa at 700 μM (1.4 wt %) compared to a G′ of 1.8±0.4×10² Pa for a sequence having simply the dA₃₀ region at the same molar concentration (FIG. 32, a,c,d). At 4 wt % (CA included), G′ of these dA30C4v2 hydrogels reaches 3×10⁴ Pa, values well outside those reported for previous unmodified DNA hydrogels.^{[9, 16]} The moduli are now more similar to conventional polymer hydrogels, with the 1.4w % hydrogel being equivalent to an 8% poly(acrylamide) gel with 1.8% N,N′-methylenebis(acrylamide) crosslinker synthesized at 20° C.^{[17]} Due to the predictability of DNA self-assembly from its sequence, it was possible to design the crosslinks with different motifs, such as the regular double-stranded helix (dsDNA₁₃, dsDNA₂₀), the i-motif (C₄, C₈) or the G-quadruplex (G4, G8) (FIG. 32, c), the formation of which is supported by circular dichroism spectroscopy (CD) (FIG. 38). No large differences were noticed between the different lengths of the crosslinking regions or crosslinking type, even if there is an increase in mean modulus in the order i-motif<G-quadruplex<double-stranded DNA, (FIG. 32, c) it is within statistical uncertainty (FIG. 46-50). While more experiments are needed to elucidate the structural basis of such high moduli, AFM and SEM imaging suggests the material easily forms hierarchical bundles reminiscent of the strong fibers in protein hydrogels (FIG. 1d-e).^{[18]} The ability to easily control crosslinking from the sequence of DNA provides the material with a second convenient handle to tune the mechanical properties, and can push those surprisingly high for DNA hydrogels.

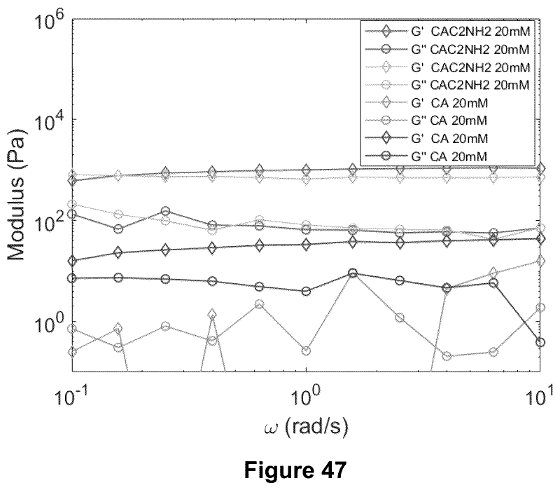

Cyanuric acid sidechain. The third unique modular handle on the hydrogel is the sidechain of the small molecule cyanuric acid (FIG. 32, d). It was previously demonstrated that CA only needs 2 faces to bind adenine, leaving the third face to display a sidechain on the triplex exterior.^{[14a]} In this previous work, it was observed that the substitution pattern on this outward face strongly affected the assembly of the fibers, both energetically and structurally. It was thus hypothesized that sidechains could have a strong effect on the mechanical properties of the hydrogels formed by these fibers. Substituting CA for its short-tailed amino derivative CAC2NH₂, which was previously shown to stiffen the motif, increased the storage modulus of pure dA₁₅ hydrogels by two orders of magnitude from 10¹ Pa to 10³ Pa at only 0.7 wt % (including CAC2NH₂), confirming the important role the sidechain has on assembly (FIG. 32, e, left, FIG. 47). The stiffness of these hydrogels at such low wt % is closer to the peptide-based hydrogels rather than conventional DNA hydrogels.^{[18a, 19]} It is believed that many factors contribute to this large increase. First, the CAC2NH₂ carries a positive charge in these conditions, reducing the electrostatic repulsion between the negatively charged DNA fibers and favoring bundling between strands. In addition, it has been shown using molecular dynamics simulations that the CAC2NH₂ derivative reduces motion in the dA/CA motif, possibly leading to stiffer fibers.^{[14a]} The CAC2NH₂ derivative does however have a slightly lower affinity to poly-dA than its parent compound, probably leading to more vacancies in the triplex at the same concentration and working against its otherwise strengthening effect.

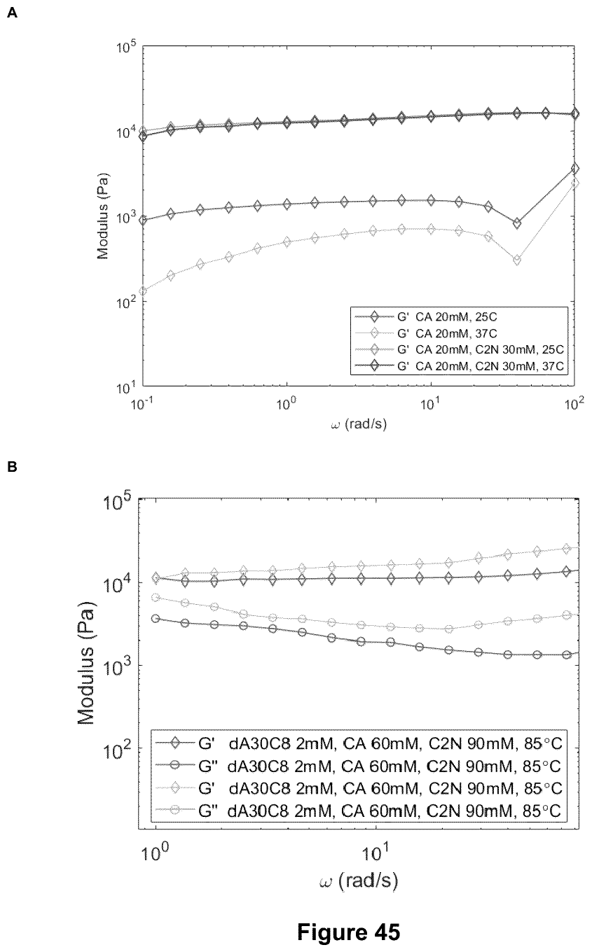

By combining the above lessons on the different handles of the hydrogels, a stiff hydrogel with a poly-dA length of 30 nucleotides was made, crosslinked by an i-motif (dA₃₀C₈) in a mixture of CA and CAC2NH₂. Joining all these handles together, the resulting hydrogel has a G′ in the range of 10⁴ Pa at only 1.7 wt % (FIG. 32, e, right). At 4.7 wt % (CA-R included), it has a G′ of 8±1×10⁴ Pa at 1 Hz, and 1±0.1×10⁵ Pa at 10 Hz. According to current knowledge, this is the highest storage modulus of any hydrogel made from unmodified DNA.^{[2b,2c,7b,16,20]} It even surpasses many hydrogels incorporating modified DNA, double-networks or other alterations to make the materials stronger.^{[21]} It also possesses high thermal stability, the 1.7 wt % gel keeping its moduli virtually unchanged at 37° C. (FIG. 45, A) and the 4.7 wt % gel retaining a storage modulus above 10⁴ Pa even at 85° C. (FIG. 45, B), and has qualitatively been observed staying a gel at 100° C.

Taken together, these independent handles allow tuning the mechanical properties of the hydrogel over a large G′ window from 10¹ Pa (i.e. dA₅₀ 420 μM, CA 20 mM) to 10⁵ Pa (i.e. dA₃₀C₈ 2 mM, CA 60 mM, CAC2NH₂ 90 mM) by using the base-pairing properties of DNA combined with the unique nature of the small molecule-mediated dA/CA motif.

Self-healing and thixotropy. An important property in supramolecular polymers is their ability to self-heal. Self-healing is especially important for hydrogels, as it allows them to heal regions broken by cell migration^{[22]} or to adapt their shapes as they are inserted or injected in their desired site of interest for drug delivery^{[23]} or 3D printing.^{[24]}

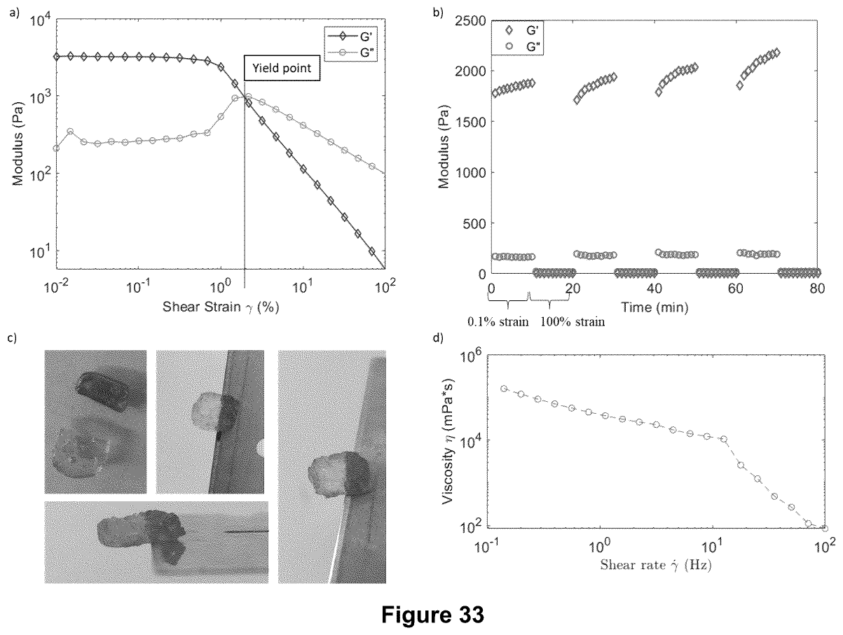

It was found that the hydrogel exhibits apparent self-healing abilities, with complete return to its initial modulus nearly instantly (<1 min.) following a shear-induced breakage. This can be done repeatedly for at least 4 cycles without any indication of weakening. Moreover, gels cut with a razor blade exhibited clear healing behavior when the cut surfaces were brought back together (FIG. 33, c). The self-healing ability likely derives from the supramolecular nature of the network, in which the reversibility of bonds holding the DNA nanofibers together allows them to break and reform without lasting damage.^{[22,25]} The slight increase in modulus after each cycle is likely caused by drying over the course of the 1 h experiment.

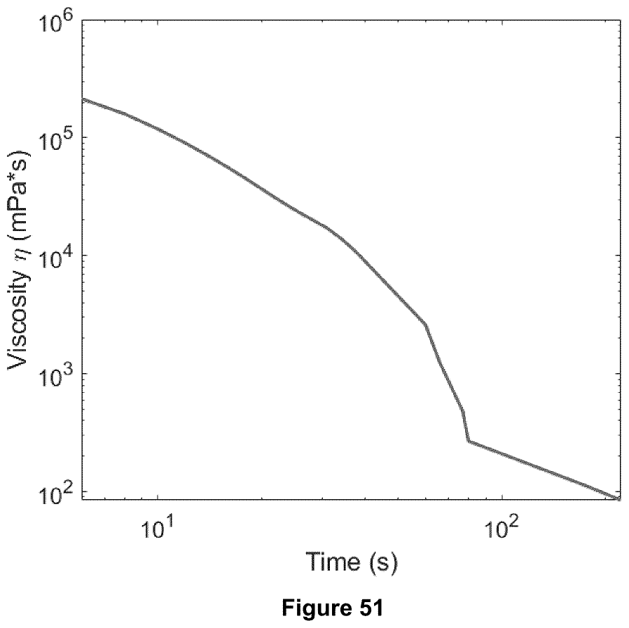

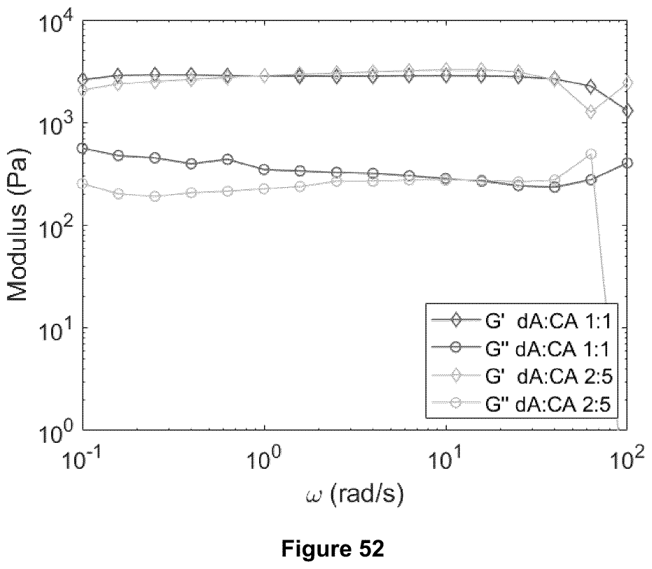

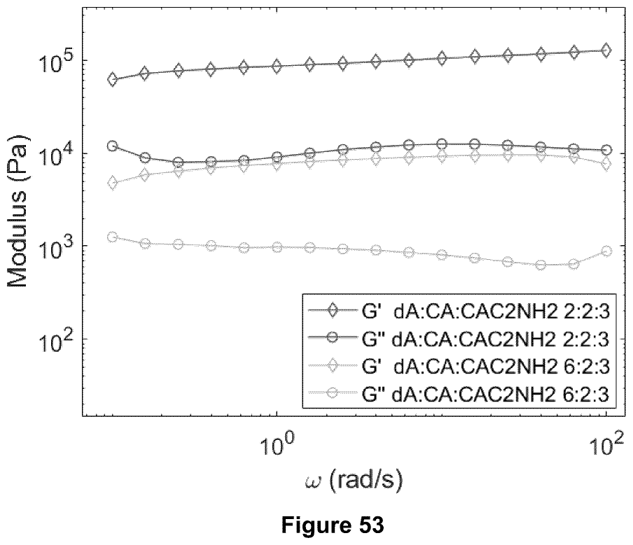

In addition to exhibiting self-healing behavior, these hydrogels exhibited thixotropic properties—the ability to turn more fluid above a certain rate of shearing—above a shear rate of 10 Hz (FIG. 33, d, FIG. 51). Thixotropy allows these hydrogels to be injected as easily as a liquid, but in its destination act as a solid, an important property for 3D printing and therapeutic delivery.

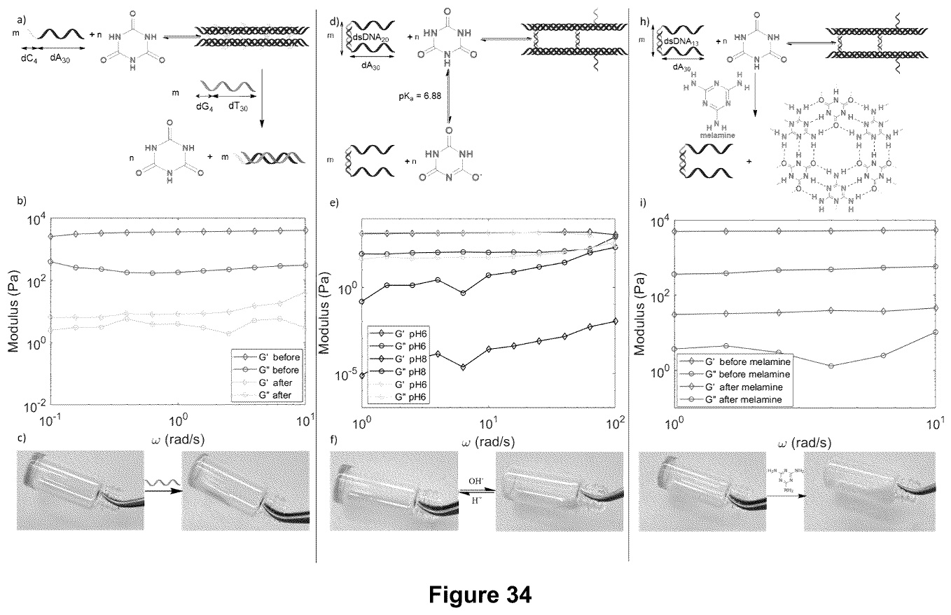

Stimuli-Responsiveness. A smart material includes materials that are able to sense and actuate functions, requiring stimuli-responsiveness.^{[26]} As the fibers making the dA/CA hydrogels are assembled in a supramolecular manner, they can revert to the free components CA and DNA in a dynamic equilibrium (FIG. 34). By tuning the availability of these components, the formation of fibers can be favored or disfavored, thus assembling or disassembling the hydrogel in response to stimuli.

Strand displacement. By adding a preferred binding partner to the DNA strand constituting the hydrogel, such as the fully complementary DNA, the equilibrium is shifted away from the fibers. In a hydrogel containing dA₃₀C₄, the addition of the complementary strand dG₄T₃₀ leads to a large loss of G′ and G″ (FIG. 34, a-c). This competition by an invading strand binding stronger than the dA/CA motif is similar to strand displacement, only the strand now displaces small molecules. This variation on the concept could be used to release a relatively large number of small molecules, some perhaps bearing therapeutic cargo, with a strand trigger. It is also an example on how the material conserves the valuable characteristics of DNA in this new hybrid assembly, while adding new characteristics based on the small molecule.

Small-molecule displacement. A competing binder can also be added to the small molecule component of the gel. One binding partner of cyanuric acid is melamine, a molecule which allows maximum hydrogen bond formation with CA to form a hexameric rosette sheet-like crystalline structure. Consequently, adding stoichiometric amount of melamine to a dA₃₀-dsDNA₁₃ hydrogel greatly decreases G′ and G″ (FIG. 34, i). As melamine rips CA away from adenine in extended 2D hydrogen bonded networks, a solid precipitates from solution as white flakes, leaving behind a liquid containing the DNA strands (FIG. 34, j). This mirrors the previous strand-displacement experiment, where now a small molecule trigger can release DNA strands into solution, where they could perform therapeutic^{[29]}, structural^{[7b, 30]} or computational tasks.^{[31]}

pH changes. In addition to displacement by melamine, the inherent slight acidity of CA (pKa=6.9) makes the hydrogels pH-responsive. Since only the protonated form of CA can form the hydrogen bonds necessary for fiber formation, the hydrogel dissolves in basic pH (FIG. 34, g-i), dissociating both the strands and the CA. pH changes are readily controllable and reversible, and when the solution is again lowered to pH 6, the mechanical properties are nearly entirely recovered in only 2 minutes. Since the pH responsiveness happens around biological pH, it is possible to use the hydrogel as a delivery agent, unforming and releasing its cargo when added to biological samples.

Encapsulation, integration and release. One application of hydrogels lies in cargo encapsulation and release. In this case, cargo can either be physically entrapped in the gel, or it can be integrated into the gel itself, by conjugating it to a poly-dA strand.

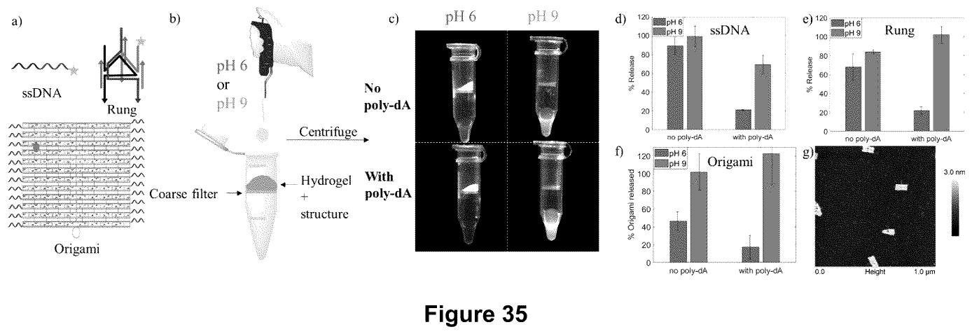

DNA origami and wireframe structures. One of the advantages of using DNA hydrogels is that they allow the seamless integration of DNA nanostructures for therapeutic or diagnostic applications. While simple DNA motifs have been previously used in hydrogels, full DNA nanostructures have not been incorporated as integral parts of DNA hydrogels. ADNA triangular wireframe assembly, as well as a DNA origami rectangle, was constructed with and without extra dA₃₀ polyadenine overhangs (FIG. 35, a). These pre-assembled structures were added to dA₃₀C₄ hydrogels one minute after they were cooled down to room temperature, to allow their incorporation or encapsulation during the polyadenine/CA assembly without denaturing their own structures.

To facilitate tracking of the DNA nanostructures, they each contained a strand modified with a cyanine-3 dye. The hydrogel encapsulating DNA nanostructures were placed over a filter in a centrifuge tube, and first washed the structure with acidic buffer with centrifugation (FIG. 35, b). Using this eluent, almost all the DNA strand and DNA wireframe structures without poly-dA overhangs were collected, while a lesser amount of the larger DNA origami was released, consistent with their physical encapsulation within the gel. On the other hand, only a small fraction of the DNA nanostructures with poly-dA overhangs was collected, showing that these structures were stably integrated within the gel. By increasing the pH of the eluting buffer, the DNA nanostructures with or without poly-dA overhangs were collected in their entirety, consistent with disintegration of the hydrogel structure at higher pH and release of the encapsulated cargo (FIG. 35, c,d,e,f). AFM imaging of the eluent showed that the DNA origami structures maintained their integrity after release from the hydrogel (FIG. 35, g). Thus, DNA nanostructures can be stably encapsulated within the DNA hydrogel at slightly acidic pH, and they can be released from the gel in a pH-responsive manner. DNA nanostructures can be used in detection,^{[32]} computation,^{[33]} reaction control^{[34]}, as well as proposed components of future therapeutics. Controlling their encapsulation and release kinetics through simple sequence modification will be valuable for spatiotemporally tuning their availability in different environments.

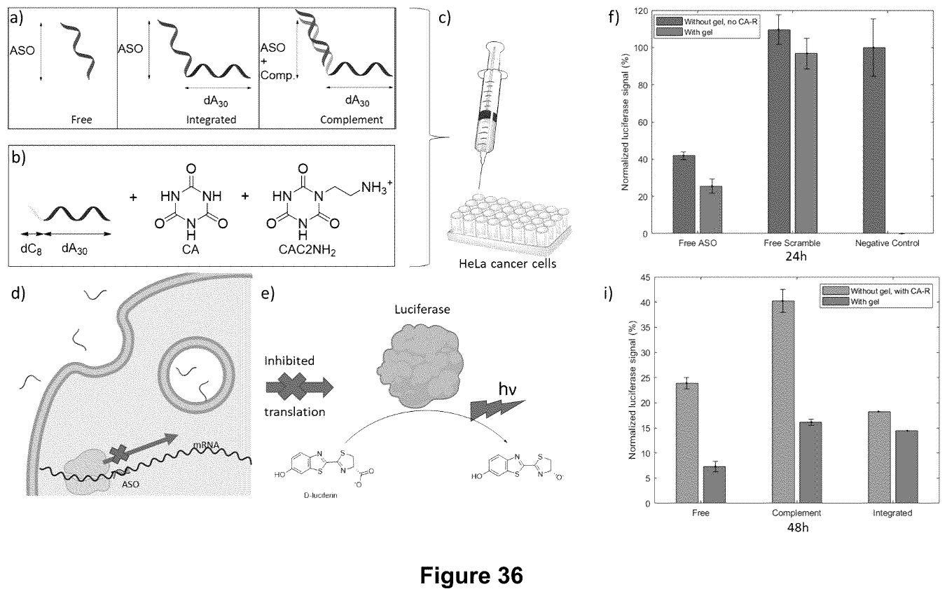

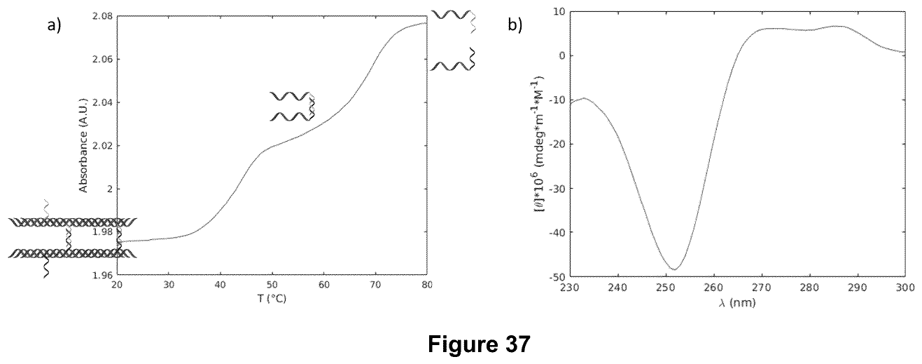

Release of antisense oligonucleotide and gene silencing in-vitro. Having shown the controlled pH release of a variety of potential nucleic acids, the hydrogels were applied to test in vitro delivery of a nucleic acid therapeutic cargo. An antisense oligonucleotide (ASO), capable of silencing the reporter luciferase gene in a Hela (cervical cancer) cell line, was chosen.^{[36]} Antisense oligonucleotides are a versatile class of therapeutics, which are applied to treat diseases such as Duchenne muscular dystrophy and spinal muscular atrophy,^{[37]} with a number of other diseases currently investigated in 29 active or recruiting clinical trials as of 2022.^{[38]} They benefit from modified bases such as the 2′-fluoroarabinose (FANA) modification, which both improves nuclease stability, binding to the target mRNA and recruitment of RNase H for degradation, resulting in high gene silencing ability.^{[39]} The DNA hydrogels can assist in the local delivery of nucleic acid therapeutics such as ASO to tissues by establishing a high local concentration, while reducing the problems associated with therapeutic levels of strands administered systemically. To load the FANA-DNA (ASO) in the gel, three different methods were used, (i) encapsulation of the free ASO strand (Free) in an unmodified dA/CA hydrogel, (ii) hybridization of the ASO to a dA/CA hydrogel equipped with overhangs complementary to the ASO sequence (Complement) and (iii) integration of the ASO into the dA/CA fibers of the hydrogel by conjugation of a dA₃₀ tail to this ASO strand (Integrated) (FIG. 36, a, Table 11). The gel itself was composed of dA₃₀C₈ strands at 700 μM, CA at 20 mM and CAC2NH₂ at 30 mM. This composition was chosen for its temperature-independent modulus, ensuring the release would be through pH and not heating (FIG. 45, A). The hydrogel was loaded into syringes and injected onto a plate of Hela cells in Dulbecco's Modified Eagle Medium supplemented with 10% Fetal Bovine Serum (DMEM) buffer. Over time, its dissolution in presence of the cell environment pH released the FANA-DNA ASO sequences. No additional transfection agents are used in this assay to decipher effects stemming from the gel itself, so a final concentration of silencing strand of 5 μM is used for gymnosis-based entry. A scramble sequence of the ASO was used as a control, while the efficiency of silencing was compared to the same strands added only with buffer either without or with CA-R (FIG. 36, f, i respectively). Silencing ability is measured by luminescence caused by the luciferase enzyme and is normalized to the number of live cells with a cell titer blue assay.

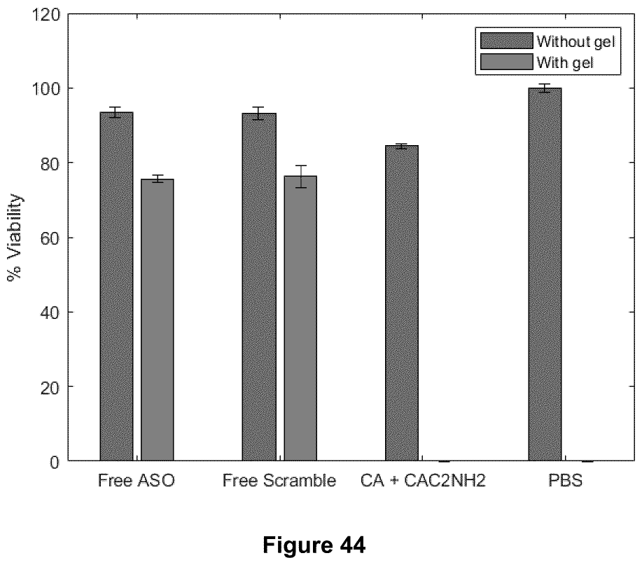

Silencing efficiency of the strands after 48 hours varied between 84±1% and 93±1% depending on the method of incorporation, with the free strand physically encapsulated in the gel having the highest activity, and the complementary strand the lowest, possibly because of the competition in binding between the complement to the ASO and the mRNA (FIG. 36, i). While the goal was to obtain a similar silencing efficiency as the free strands in buffer, only locally released, it was surprising to find a stronger silencing effect when the strands were encapsulated in the hydrogels (FIG. 36, f). Results were normalized to cell viability, with the ASO-loaded hydrogels showing similar levels of cell growth to the non-loaded hydrogels, which was in turn lower than cells grown in media (FIG. 44). It was verified that this is not an effect of the cyanuric acid molecules added, as ASO strands in presence of these molecules, without the gel, are less effective at gene silencing (FIG. 36, i). It is hypothesized that increased local concentration around the gel, interactions of the gel with cell membranes, and/or increased endosomal escape may explain the improved silencing ability of the hydrogel/ASO system.

### CONCLUSION

This work has developed an injectable DNA-based hydrogel able to act as a local delivery vehicle for oligonucleotides, and to encapsulate and release DNA origami and wireframe nanostructures, selectively triggered by physiologically relevant pH. Key to this strategy is the use of a newly discovered DNA motif, where poly-dA strands can be assembled into supramolecular fibers with the small non-toxic molecule, cyanuric acid (CA). The hydrogel can be formed by crosslinking these fibers using canonical or non-canonical DNA base-paring: this poly-dA/CA gel is unique in that it contains a high density of integrated small molecules along its backbone. Using this strategy, hydrogels were made with unmodified DNA with a range of moduli from 10¹-10⁵ Pa, making them the stiffest unmodified DNA hydrogels by two orders of magnitude. This large mechanical space can be navigated not only by tuning the DNA sequence but also by controlling the modifications of the small molecule cyanuric acid binding partner. The presence of the cyanuric acid imbues the material with inherent stimuli-responsiveness to specific DNA sequences, pH and small molecules such as melamine. The supramolecular nature of the interaction allows for efficient, fast self-healing and thixotropy, which make it easy to use for injections of therapeutics. The hydrogel still retains the attractive advantages of DNA, with its stimuli responsiveness to strand displacement reactions and its easy integration of DNA nanostructures. DNA wireframe assemblies and DNA origami can be incorporated within the hydrogel either via physical encapsulation, or through the attachment of polyadenine overhangs to these DNA nanostructures and integration into the poly-dA/CA fibers. This ease of integration allowed the incorporation of a therapeutic nucleic acid—an antisense oligonucleotide (ASO) for luciferase-into the structure, and to release it in the presence of the physiological pH of cells. It was found that, surprisingly, the presence of the hydrogel itself improved the silencing efficiency by a factor of 2-3 compared to the ASO in buffer, reaching a 95±1% of silencing after 48 hours under optimized conditions.

The combination of thixotropy, tunable stiffness, self-healing behavior, stimuli-responsiveness to pH and enhanced silencing observed in presence of this gel make it a promising material for future development of a local nucleic acid release system. The ability of CA to be functionalized with an amino group in the hydrogel could also allow attachment of drugs held in the hydrogel for multi-drug stimuli-responsive therapy.

### Experimental Section/Methods

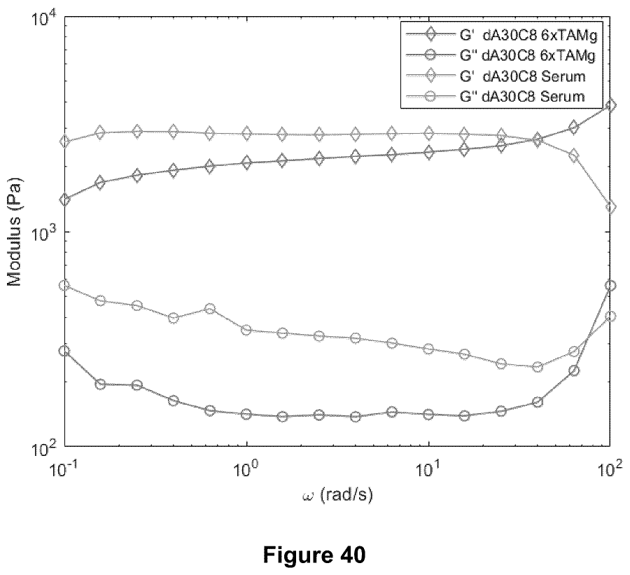

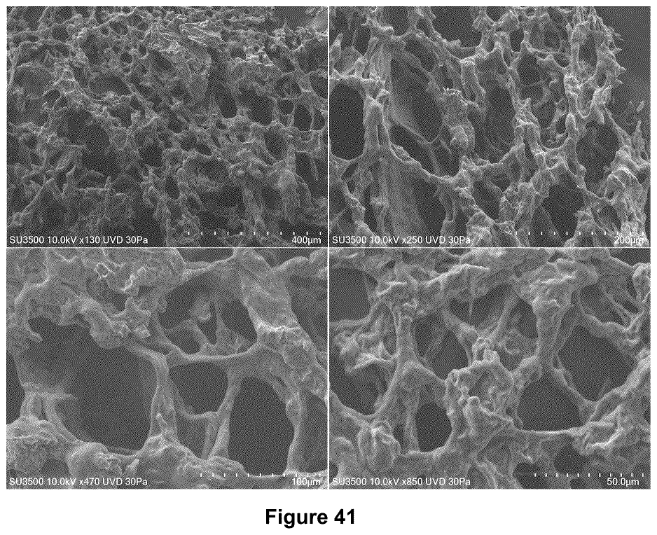

Hydrogel Assembly and Rheology: Unless otherwise stated, the preparation of gels follows this procedure. The appropriate amounts of strands are evaporated from their stocks on a speedvaccuum in a 300 μL Eppendorf. For the dA₃₀-dsDNAₙ hydrogels, the concentration is half strand1 and half strand2. Once dry, 50 μL or 100 μL of a buffer containing the desired concentration of CA and/or CAC2NH₂ is added to the Eppendorf. Two types of buffers were used in this work: the Mag buffer (MgCl₂ 45.6 mM, Tris 240 mM, adjusted to pH 6 after CA addition by glacial acetic acid) and the Serum buffer (NaCl 140 mM, KCl 5 mM, Ca(NO₃)₂ 2.5 mM, MgCl₂ 1.5 mM, MES 10 mM, adjust to pH 6 after CA addition with HCl). Both buffers give similar gels rheologically (FIG. 40). For concentrations of CA exceeding 20 mM or CAC2NH₂ 30 mM, the excess CA-R is dried with the strands from a water/CA 20 mM solution adjusted to pH 6. The dried DNA is mixed and sonicated to help it dissolve, forming instantly a cloudy filamentous gel. To homogenize the gels, the Eppendorf is heated to 85° C. for 5 minutes a Bio-Rad T₁₀₀TM thermocycler, then cooled down to 20° C. and kept at that temperature overnight, unless otherwise noted. They are measured the next day. It is important to properly homogenize the gels, mixing them at high temp if needed, and to quickly cool them, or they may form more localized aggregates with varying rheology. Due to the high melting temperature of dA₃₀C₈ 2 mM, CA 60 mM, CAC2NH₂ 90 mM, the gel was prepared by heating at 100° C. for 20 min (sealed to prevent water escaping), before being brought to 20° C. Clear monophasic gels are formed from this process.

TABLE 7
Sequences of DNA strands used for hydrogels.

| Name | Sequence (5′→3′) | SEQ ID NO |
|---|---|---|
| dA₁₅ | AAAAAAAAAAAAAAA | 5 |
| dA₃₀ | AAAAAAAAAAAAAAAAAAAAAAAAAAAAAA | 6 |
| dA₅₀ | AAAAAAAAAAAAAAAAAAAAAAAAAAAAAAAAAAAAAAAAAAAAAAAAAA | 28 |
| dA₃₀C₄ | AAAAAAAAAAAAAAAAAAAAAAAAAAAAAACCCC | 9 |
| dA₃₀C₈ | AAAAAAAAAAAAAAAAAAAAAAAAAAAAAACCCCCCCC | 8 |
| dA₃₀G₄ | AAAAAAAAAAAAAAAAAAAAAAAAAAAAAAGGGG | 29 |
| dA₃₀G₈ | AAAAAAAAAAAAAAAAAAAAAAAAAAAAAAGGGGGGGG | 30 |
| dA₃₀-dsDNA₁₃-strand1 | AAAAAAAAAAAAAAAAAAAAAAAAAAAAAACTAGAAGTGTCCAGTTA | 31 |
| dA₃₀-dsDNA₁₃-strand2 | AAAAAAAAAAAAAAAAAAAAAAAAAAAAAATTACTAACTGGACACTT | 32 |
| dA₃₀C₄v₂-strand1 | AAAAAAAAAAAAAAAAAAAAAAAAAAAAAACCCCATTCTACTTGAGAGAGCGAC | 33 |
| dA₃₀C₄v₂-strand2 | AAAAAAAAAAAAAAAAAAAAAAAAAAAAAACCCCGTCGCTCTCTCAAGTAGAAT | 34 |

Rheological measurements: Rheological properties were measured using stress-controlled rheometer (MCR 302, Anton Paar) using a 15 mm diameter cone plate at 0.2 mm (50 μL gels) or 0.5 mm (100 μL gels) gap distance. Samples were taken directly from the Eppendorf in which they were formed by prying it open with a razor blade and placed under the cone plate. The temperature was maintained at 25° C. for all measurements using a Peltier temperature control unit unless otherwise noted. Due to the small thickness of the samples, no temperature gradients are expected. To avoid drying, a solvent trap was used in all the measurements. Amplitude sweeps were first conducted to assess the linear viscoelastic range of the samples by slowly increasing the strain from 0.01% to 100%. The yield point was found to be around 1%. Oscillatory frequency sweep tests were measured between 0.1 Hz and 100 Hz at 0.1% strain. Strain-cycle experiment was done by cycling the gel between 0.1% and 100% strains at 1 Hz. To understand the shear-thinning behavior of the samples, the viscosity was measured at increasing strain rate ranging from (0.1-100 Hz).

### Stimuli Responsiveness:

A) Strand complement. Two 50 μL dA₃₀C₄ 700 μM hydrogel in Mag buffer with CA 20 mM samples were prepared, as well as a 10 μL sample of dG₄T₃₀ 3 mM (1.5 eq.) in the same buffer and CA concentration. A sample of dA₃₀C₄ hydrogel was measured independently. The dG₄T₃₀ sample was added to the second dA₃₀C₄ sample and vortexed in its Eppendorf for 2 minutes before being measured.
B) Melamine. A 50 μL hydrogel composed of dA₃₀-dsDNA₁₃ 700 μM and CA 20 mM in Mag buffer was measured in the rheometer. The plate of the rheometer was lifted and 50 μL of solution of 20 mM melamine in Mag buffer at pH6 was added to it. Instant white precipitate formed and was encouraged by gently mixing with a spatula for 30s before the resulting liquid with solid precipitate was measured rheologically.
C) pH. A volume of 180 μL of dA₃₀C₄v2 700 μM CA 20 mM hydrogel in Mag buffer was prepared. The initial pH was measured at 5.71 with a surface pH meter (Mettler Toledo). An approximate volume of 50 μL was separated and measured rheologically. To the remaining 130 μL, 2.70 μL of NaOH 5.04M was added and stirred through vortexing. The resulting pH of the liquid was measured at 7.91. 50 μL of it were measured rheologically. To the remaining 80 μL, 1.5 μL of HCl 5.04M was added by aliquot and mixed through vortexing, forming a gel with a pH of 5.66 after 2 minutes. To note that in general, it is important to mix vigorously with the addition of the acid, or localized gels will form rather than a homogeneous one. That gel was then analyzed rheologically.

SEM and AFM imaging: A 100 μL dA₃₀-dsDNA₁₃ 700 μM in CA 20 mM Mag buffer was prepared. It was frozen with liquid nitrogen then dried in freeze-drier overnight. The dried sample was placed on carbon tape and imaged by Hitachi SU3500 scanning electron microscope equipped with ultra-variable pressure detector operating at 10 kV accelerating voltage under 30 Pa pressure.

A hydrogel with the same composition was broken down by adding 3 mL of Mag buffer and strongly mixing with the pipette, then 5 μL of the sample was dropped onto a freshly cleaved mica surface for 30 seconds, followed by wicking off most of the liquid from the mica surface using a filter paper. The mica surface was then further dried under a stream of compressed air for 30 seconds before it was put under vacuum for at least 2 hrs prior to imaging. AFM images were acquired in ScanAsyst mode under air conditions on a Multimode 8 Scanning Probe Microscope from Bruker with a Nanoscope V controller equipped with a ScanAsyst-Air silicon tip on nitride lever (tip radius=2 nm, k=0.4 N/m, fₒ=70 KHz; Bruker).

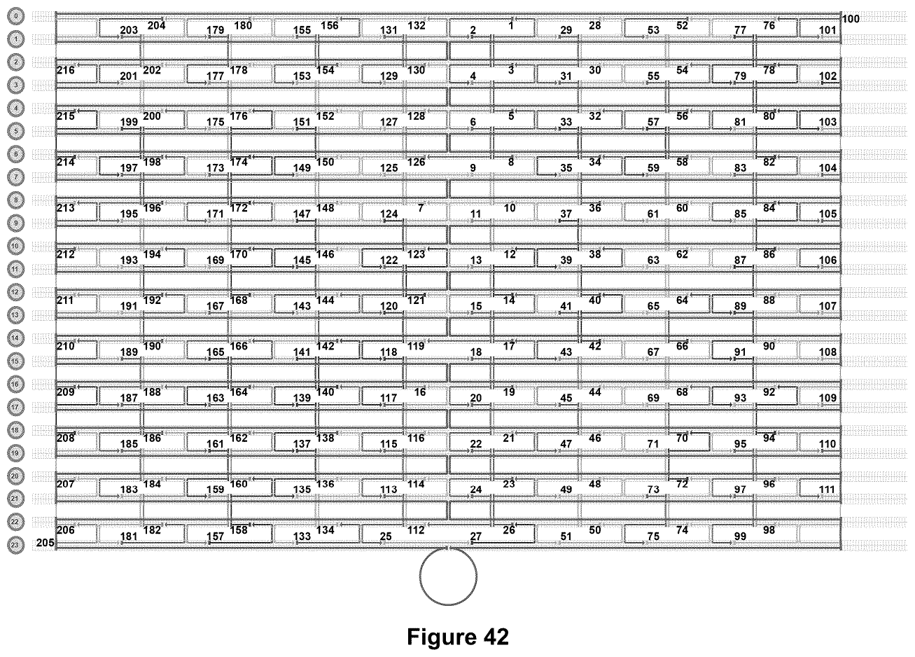

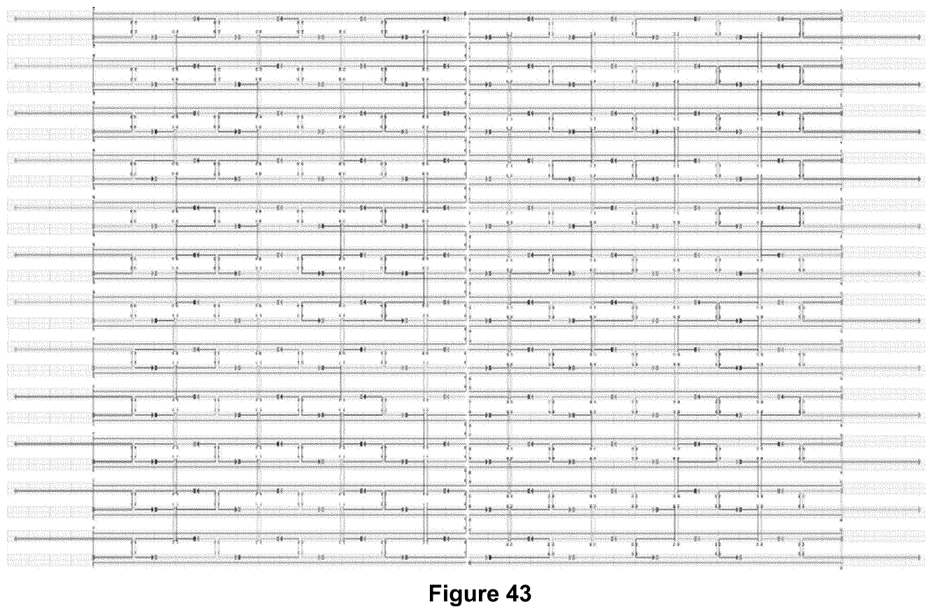

Nanostructure Assembly, Encapsulation, Integration and Release: The design and assembly of the rectangle DNA origami structure functionalized with poly-dA strands were based on the method reported by Rothemund et al.^{[41]}, while the wireframe rung was based on the method reported by Rahbani et al.^{[42]} For the origami, the long circular single-stranded viral scaffold M13mp18 (purchased from Guild BioSciences, USA) was folded into a rectangular tile with the aids of short single-stranded staple strands (Table 9 and 10, FIGS. 42 and 43, purchased from Bioneer, Inc., USA). A single staple strand was synthesized on a Mermade MM6 synthesizer with a 3′-Cy5 dye. The origami structure was assembled in one-pot, where 3 nM M13mp18 scaffold and 45 nM each staple strands were mixed and heated to and held at 95° C. for 5 min in 1×TAMg buffer (pH=8.3) and slowly annealed to 20° C. (−1° C./min). To remove excess staple strands and adapt the solution pH to 6.7 (compatible with dA/CA fiber formation), the annealed sample was washed in a 100 kDa Amicon centrifugal filter Unit. First, 500 μL samples were centrifuged at 6500 rpm at 4° C. for 5 min. Then, 400 μL 1×TAMg (pH=6.7) was added and the samples were centrifuged again at 5000 rpm at 4° C. for 6 min. This filtration step was repeated four more times. Approximately 50-100 μL samples were recovered after the purification and were quantified by a NanoDrop Lite Spectrophotometer (ThermoFisher Scientific).

For the wireframe rung, it is formed by mixing all component strands either with or without poly-dA₃₀ overhangs (Table 8) in equimolar concentrations to a final concentration of 0.30 μM in 1×TAMg. A single strand per construct contained a 3′-Cy3 dye. The mixture was annealed from 95° C. to 20° C. over 6 h resulting in a quantitative yield of the rung.

A 100 μM hydrogel of dA₃₀C₄ 500 μM (CA 20 mM, Mag buffer) was brought to 85° C. for 5 min, and cooled to 20° C. for 1 min, then the constructs are added for a final concentration of 1 nM (Origami) or 40 nM (rung and ssDNA) and left to cool at 20° C. for 15 min. The gels are then placed on a filter of a Freeze 'N Squeeze™ DNA Gel Extraction Spin Columns (BioRad). Either 100 μL of Mag buffer at pH 6 or pH 9 is added to them for 25 min, then the column are spin down at 6 k for 6 min. The filtrate is collected, mixed gently, and analyzed by fluorescence of the Cy3 or Cy5 dye in triplicate on a Biotek Synergy HT plate reader. The concentration is obtained through a calibration curve. An origami filtrate from the pH 9 was collected and 5 μL of it was dropped on freshly cleaved mica and imaged on AFM.

In vitro silencing and viability assays: Hydrogels were formed using the sequences in Table 11. The ASO, Scramble, ASO-integrated and Scramble integrated are synthesized using a Mermade MM6 synthesizer from Bioautomation, then purified using gel-electrophoresis. The ASO-Complement, Scramble-Complement and dA₃₀C₈ were all purchased from Integrated DNA Technologies desalted and used as is. FANA-DNA strands were synthesized in house as described elsewhere.^{[43]}

Hydrogels were prepared by using a CA 20 mM, CAC2NH₂ 30 mM Serum buffer adjusted to pH 6, sterilized by filtration through a 0.2 μm Nylon Centrifugal Filters from Canadian Life Sciences. The gelling strands of dA₃₀C₈ were at 700 μM, the therapeutic strands (ASO, Scramble, ASO-integrated or Scramble-Integrated) at 15 μM, and, for the complement samples, the complement strands (ASO-complement, Scramble-Complement) at 30 μM in a total volume of 100 μL. They were heated to 85° C. for 20 minutes before being aspirated into 1 mL syringes while hot. The syringes were left at room temperature for 1 h before being used.

Luciferase expressing Hela cells were maintained in DMEM containing 10% FBS supplemented with antibiotic/antimycotics at 37° C., 5% CO2. Cells were passed every 3 days in a ratio of 1:5. Luciferase knockdown assays were performed by plating 50000 cells per well, for 24 hour experiments, or 25000 cells per well, for 48 hour experiments, in a 24 well plate. Cells were incubated overnight at 37° C., 5% CO₂ to allow for adhesion to the plate. Following incubation, 100 μL of either hydrogel or buffer solutions were added in duplicate (48 h) or quadruplicate (24 h) to wells directly, as well as 200 μL of fresh DMEM with 10% FBS. Cells were then incubated for either 24 or 48 hours post addition of samples.

Cytotoxicity and cell viability was analyzed by incubating cells with using 100 μL of a premade fluorescent reagent (Celltiter Blue) for 2 hours at 37° C., 5% CO2. Fluorescence from the 24 well plates was then measured Ex. 530 nm, Em. 590 nm using Biotek Synergy HT, using BioTek Gen 4 Software (FIG. 44).

Luciferase assay was performed by first removing media from cells then adding 200 μL of a 1:1 Mix of Promega Glo-Lysis Buffer and Promega Bright-Glo Luciferase Assay System to each well. Luminescence was measured at 528 nm using Biotek Biotek Synergy HT, using BioTek Gen 4 Software. Luciferase silencing was corrected with cell viability and normalized to cells with a simple 1×PBS addition.

### Example 4-DNA Hydrogel Wireframe Structures

The strands were purchased desalted from Integrated DNA Technologies (IDT) unless otherwised noted. Cyanuric acid (CA)₉₈%, 4-morpholineethanesulfonic acid hydrate (MES), MgCl₂*6H₂O, Ca(NO₃)₂*2H₂O, KCl, NaCl, tris (hydroxymethyl)aminomethane (Tris), glacial acetic acid, urea, EDTA were obtained from Sigma-Aldrich. CAC2NH₂ was synthesized as previously described.

Cyanine 3 (Cy3) and Cyanine 5 (Cy5) phosphoramidite were purchased from GlenResearch. Dulbecco's Modified Eagle Medium (DMEM) and phosphate-buffered saline (PBS) were purchased from Life Technologies. Fetal bovine serum (FBS), 0.05% Trypsin-EDTA, sodium pyruvate were obtained from Wisent Bioproducts. Bright-Glo Luciferase assay and Cell-Titer Blue assay was obtained from Promega. The luciferase-expressing HeLa X1/5 cells are a generous gift from Dr. Pelletier (McGill).

Circular Dichroism (CD) were performed on JASCO J-810 spectropolarimeter equipped with a Peltier temperature controller, a xenon lamp and a water recirculator with a 0.1 mm path length quartz cuvette. UV-vis measurements were performed on a Cary 300 Series UV-Vis Spectrophotometer from Agilent connected to a water recirculator with a 1 mm quartz cuvette. Quantification of DNA and RNA were performed with a Nanodrop Lite spectrophotometer from Thermo Scientific.

TABLE 8
Oligonucleotide sequences used in the assembly of wireframe rung unit.
The triangular rung unit is formed by mixing all component
strands (V, C1, C2, R1 cap polyA30, R2 cap polyA30, and R3 cap
polyA30 or V, C1, C2, R1 cap, R2 cap, and R3 cap)

| | | SEQ ID |
|---|---|---|
| Name | Sequence (5′→3′) | NO |
| V | CTCAGCAGCGAAAAACCGCTTTACAACATTCGAGGCACG | 35 |
| | TTGTACGTCCACACTTGGAACCTCATCGCACATCCGCCTG | |
| | CCACGCTCTTAGCATAGGACGGCGGCGTTAAATA | |
| C1 | CGGTGCATTTCGACGGTACTTCGTACAACGTGCCTCGAAT | 36 |
| | GTAGAGCGTGGCAGGCGGATGTGAAGCAGTTGCAGCGTA | |
| | CTCGT | |
| C2 | TCGGCAGACTAATACACCTGTCGATGAGGTTCCAAGTGTG | 37 |
| | GATAGCTAGGTAACGGATTGAGC | |
| R1 cap | AAAAAAAAAAAAAAAAAAAAAAAAAAAAAACGAGTACG | 38 |
| poly-dA₃₀ | CTGCAACTGCTACCAGGTGTATTAGTCTGCCGAAAAAAA | |
| | AAAAAAAAAAAAAAAAAAAAAAA | |
| R2 cap | AAAAAAAAAAAAAAAAAAAAAAAAAAAAAACTCAATCC | 39 |
| poly-dA₃₀ | GTTACCTAGCTCCAGTACCGTCGAAATGCACCAAAAAAA | |
| | AAAAAAAAAAAAAAAAAAAAAAA | |
| R3 cap | AAAAAAAAAAAAAAAAAAAAAAAAAAAAAAAACGCCGC | 40 |
| poly-dA₃₀ | CGTCCTATGCTTTGTAAAGCGGTTTTTCGCTGAAAAAAAA | |
| | AAAAAAAAAAAAAAAAAAAAAA | |
| R1 cap | CGAGTACGCTGCAACTGCTACCAGGTGTATTAGTCTGCCG | 41 |
| R2 cap | CTCAATCCGTTACCTAGCTCCAGTACCGTCGAAATGCACC | 42 |
| R3 cap | CGCCGCCGTCCTATGCTTTGTAAAGCGGTTTTTCGCTG | 43 |

TABLE 9
Staple strand sequences for the
origami without poly-dA.

| | SEQ |
|---|---|
| Staple Strand Sequences | ID NO |
| CAAGCCCAATAGGAACCCATGTACAAACAGTT | 44 |
| AATGCCCCGTAACAGTGCCCGTATCTCCCTCA | 45 |
| TGCCTTGACTGCCTATTTCGGAACAGGGATAG | 46 |
| GAGCCGCCCCACCACCGGAACCGCGACGGAAA | 47 |
| AACCAGAGACCCTCAGAACCGCCAGGGGTCAG | 48 |
| TTATTCATAGGGAAGGTAAATATTCATTCAGT | 49 |
| CATAACCCGAGGCATAGTAAGAGCTTTTTAAG | 50 |
| ATTGAGGGTAAAGGTGAATTATCAATCACCGG | 51 |
| AAAAGTAATATCTTACCGAAGCCCTTCCAGAG | 52 |
| GCAATAGCGCAGATAGCCGAACAATTCAACCG | 53 |
| CCTAATTTACGCTAACGAGCGTCTAATCAATA | 54 |
| TCTTACCAGCCAGTTACAAAATAAATGAAATA | 55 |
| ATCGGCTGCGAGCATGTAGAAACCTATCATAT | 56 |
| CTAATTTATCTTTCCTTATCATTCATCCTGAA | 57 |
| GCGTTATAGAAAAAGCCTGTTTAGAAGGCCGG | 58 |
| GCTCATTTTCGCATTAAATTTTTGAGCTTAGA | 59 |
| AATTACTACAAATTCTTACCAGTAATCCCATC | 60 |
| TTAAGACGTTGAAAACATAGCGATAACAGTAC | 61 |
| TAGAATCCCTGAGAAGAGTCAATAGGAATCAT | 62 |
| CTTTTACACAGATGAATATACAGTAAACAATT | 63 |
| TTTAACGTTCGGGAGAAACAATAATTTTCCCT | 64 |
| CGACAACTAAGTATTAGACTTTACAATACCGA | 65 |
| GGATTTAGCGTATTAAATCCTTTGTTTTCAGG | 66 |
| ACGAACCAAAACATCGCCATTAAATGGTGGTT | 67 |
| GAACGTGGCGAGAAAGGAAGGGAACAAACTAT | 68 |
| TAGCCCTACCAGCAGAAGATAAAAACATTTGA | 69 |
| CGGCCTTGCTGGTAATATCCAGAACGAACTGA | 70 |
| CTCAGAGCCACCACCCTCATTTTCCTATTATT | 71 |
| CTGAAACAGGTAATAAGTTTTAACCCCTCAGA | 72 |
| AGTGTACTTGAAAGTATTAAGAGGCCGCCACC | 73 |
| GCCACCACTCTTTTCATAATCAAACCGTCACC | 74 |
| GTTTGCCACCTCAGAGCCGCCACCGATACAGG | 75 |
| GACTTGAGAGACAAAAGGGCGACAAGTTACCA | 76 |
| AGCGCCAACCATTTGGGAATTAGATTATTAGC | 77 |
| GAAGGAAAATAAGAGCAAGAAACAACAGCCAT | 78 |
| GCCCAATACCGAGGAAACGCAATAGGTTTACC | 79 |
| ATTATTTAACCCAGCTACAATTTTCAAGAACG | 80 |
| TATTTTGCTCCCAATCCAAATAAGTGAGTTAA | 81 |
| GGTATTAAGAACAAGAAAAATAATTAAAGCCA | 82 |
| TAAGTCCTACCAAGTACCGCACTCTTAGTTGC | 83 |
| ACGCTCAAAATAAGAATAAACACCGTGAATTT | 84 |
| AGGCGTTACAGTAGGGCTTAATTGACAATAGA | 85 |
| ATCAAAATCGTCGCTATTAATTAACGGATTCG | 86 |
| CTGTAAATCATAGGTCTGAGAGACGATAAATA | 87 |
| CCTGATTGAAAGAAATTGCGTAGACCCGAACG | 88 |
| ACAGAAATCTTTGAATACCAAGTTCCTTGCTT | 89 |
| TTATTAATGCCGTCAATAGATAATCAGAGGTG | 90 |
| AGATTAGATTTAAAAGTTTGAGTACACGTAAA | 91 |
| AGGCGGTCATTAGTCTTTAATGCGCAATATTA | 92 |
| GAATGGCTAGTATTAACACCGCCTCAACTAAT | 93 |
| CCGCCAGCCATTGCAACAGGAAAAATATTTTT | 94 |
| CCCTCAGAACCGCCACCCTCAGAACTGAGACT | 95 |
| CCTCAAGAATACATGGCTTTTGATAGAACCAC | 96 |
| TAAGCGTCGAAGGATTAGGATTAGTACCGCCA | 97 |
| CACCAGAGTTCGGTCATAGCCCCCGCCAGCAA | 98 |
| TCGGCATTCCGCCGCCAGCATTGACGTTCCAG | 99 |
| AATCACCAAATAGAAAATTCATATATAACGGA | 100 |
| TCACAATCGTAGCACCATTACCATCGTTTTCA | 101 |
| ATACCCAAGATAACCCACAAGAATAAACGATT | 102 |
| ATCAGAGAAAGAACTGGCATGATTTTATTTTG | 103 |
| TTTTGTTTAAGCCTTAAATCAAGAATCGAGAA | 104 |
| AGGTTTTGAACGTCAAAAATGAAAGCGCTAAT | 105 |
| CAAGCAAGACGCGCCTGTTTATCAAGAATCGC | 106 |
| AATGCAGACCGTTTTTATTTTCATCTTGCGGG | 107 |
| CATATTTAGAAATACCGACCGTGTTACCTTTT | 108 |
| AATGGTTTACAACGCCAACATGTAGTTCAGCT | 109 |
| TAACCTCCATATGTGAGTGAATAAACAAAATC | 110 |
| AAATCAATGGCTTAGGTTGGGTTACTAAATTT | 111 |
| GCGCAGAGATATCAAAATTATTTGACATTATC | 112 |
| AACCTACCGCGAATTATTCATTTCCAGTACAT | 113 |
| ATTTTGCGTCTTTAGGAGCACTAAGCAACAGT | 114 |
| CTAAAATAGAACAAAGAAACCACCAGGGTTAG | 115 |
| GCCACGCTATACGTGGCACAGACAACGCTCAT | 116 |
| GCGTAAGAGAGAGCCAGCAGCAAAAAGGTTAT | 117 |
| GGAAATACCTACATTTTGACGCTCACCTGAAA | 118 |
| TATCACCGTACTCAGGAGGTTTAGCGGGGTTT | 119 |
| TGCTCAGTCAGTCTCTGAATTTACCAGGAGGT | 120 |
| GGAAAGCGACCAGGCGGATAAGTGAATAGGTG | 121 |
| TGAGGCAGGCGTCAGACTGTAGCGTAGCAAGG | 122 |
| TGCCTTTAGTCAGACGATTGGCCTGCCAGAAT | 123 |
| CCGGAAACACACCACGGAATAAGTAAGACTCC | 124 |
| ACGCAAAGGTCACCAATGAAACCAATCAAGTT | 125 |
| TTATTACGGTCAGAGGGTAATTGAATAGCAGC | 126 |
| TGAACAAACAGTATGTTAGCAAACTAAAAGAA | 127 |
| CTTTACAGTTAGCGAACCTCCCGACGTAGGAA | 128 |
| GAGGCGTTAGAGAATAACATAAAAGAACACCC | 129 |
| TCATTACCCGACAATAAACAACATATTTAGGC | 130 |
| CCAGACGAGCGCCCAATAGCAAGCAAGAACGC | 131 |
| AGAGGCATAATTTCATCTTCTGACTATAACTA | 132 |
| TTTTAGTTTTTCGAGCCAGTAATAAATTCTGT | 133 |
| TATGTAAACCTTTTTTAATGGAAAAATTACCT | 134 |
| TTGAATTATGCTGATGCAAATCCACAAATATA | 135 |
| GAGCAAAAACTTCTGAATAATGGAAGAAGGAG | 136 |
| TGGATTATGAAGATGATGAAACAAAATTTCAT | 137 |
| CGGAATTATTGAAAGGAATTGAGGTGAAAAAT | 138 |
| ATCAACAGTCATCATATTCCTGATTGATTGTT | 139 |
| CTAAAGCAAGATAGAACCCTTCTGAATCGTCT | 140 |
| GCCAACAGTCACCTTGCTGAACCTGTTGGCAA | 141 |
| GAAATGGATTATTTACATTGGCAGACATTCTG | 142 |
| TTTTTATAAGTATAGCCCGGCCGTCGAG | 143 |
| AGGGTTGATTTTATAAATCCTCATTAAATGATATTC | 144 |
| ACAAACAATTTTAATCAGTAGCGACAGATCGATAGC | 145 |
| AGCACCGTTTTTTAAAGGTGGCAACATAGTAGAAAA | 146 |
| TACATACATTTTGACGGGAGAATTAACTACAGGGAA | 147 |
| GCGCATTATTTTGCTTATCCGGTATTCTAAATCAGA | 148 |
| TATAGAAGTTTTCGACAAAAGGTAAAGTAGAGAATA | 149 |
| TAAAGTACTTTTCGCGAGAAAACTTTTTATCGCAAG | 150 |
| ACAAAGAATTTTATTAATTACATTTAACACATCAAG | 151 |
| AAAACAAATTTTTTCATCAATATAATCCTATCAGAT | 152 |
| GATGGCAATTTTAATCAATATCTGGTCACAAATATC | 153 |
| AAACCCTCTTTTACCAGTAATAAAAGGGATTCACC | 154 |
| AGTCACACGTTTT | |
| CCGAAATCCGAAAATCCTGTTTGAAGCCGGAA | 155 |
| CCAGCAGGGGCAAAATCCCTTATAAAGCCGGC | 156 |
| GCATAAAGTTCCACACAACATACGAAGCGCCA | 157 |
| GCTCACAATGTAAAGCCTGGGGTGGGTTTGCC | 158 |
| TTCGCCATTGCCGGAAACCAGGCATTAAATCA | 159 |
| GCTTCTGGTCAGGCTGCGCAACTGTGTTATCC | 160 |
| GTTAAAATTTTAACCAATAGGAACCCGGCACC | 161 |
| AGACAGTCATTCAAAAGGGTGAGAAGCTATAT | 162 |
| AGGTAAAGAAATCACCATCAATATAATATTTT | 163 |
| TTTCATTTGGTCAATAACCTGTTTATATCGCG | 164 |
| TCGCAAATGGGGCGCGAGCTGAAATAATGTGT | 165 |
| TTTTAATTGCCCGAAAGACTTCAAAACACTAT | 166 |
| AAGAGGAACGAGCTTCAAAGCGAAGATACATT | 167 |
| GGAATTACTCGTTTACCAGACGACAAAAGATT | 168 |
| GAATAAGGACGTAACAAAGCTGCTCTAAAACA | 169 |
| CCAAATCACTTGCCCTGACGAGAACGCCAAAA | 170 |
| CTCATCTTGAGGCAAAAGAATACAGTGAATTT | 171 |
| AAACGAAATGACCCCCAGCGATTATTCATTAC | 172 |
| CTTAAACATCAGCTTGCTTTCGAGCGTAACAC | 173 |
| TCGGTTTAGCTTGATACCGATAGTCCAACCTA | 174 |
| TGAGTTTCGTCACCAGTACAAACTTAATTGTA | 175 |
| CCCCGATTTAGAGCTTGACGGGGAAATCAAAA | 176 |
| GAATAGCCGCAAGCGGTCCACGCTCCTAATGA | 177 |
| GAGTTGCACGAGATAGGGTTGAGTAAGGGAGC | 178 |
| GTGAGCTAGTTTCCTGTGTGAAATTTGGGAAG | 179 |
| TCATAGCTACTCACATTAATTGCGCCCTGAGA | 180 |
| GGCGATCGCACTCCAGCCAGCTTTGCCATCAA | 181 |
| GAAGATCGGTGCGGGCCTCTTCGCAATCATGG | 182 |
| AAATAATTTTAAATTGTAAACGTTGATATTCA | 183 |
| GCAAATATCGCGTCTGGCCTTCCTGGCCTCAG | 184 |
| ACCGTTCTAAATGCAATGCCTGAGAGGTGGCA | 185 |
| TATATTTTAGCTGATAAATTAATGTTGTATAA | 186 |
| TCAATTCTTTTAGTTTGACCATTACCAGACCG | 187 |
| CGAGTAGAACTAATAGTAGTAGCAAACCCTCA | 188 |
| GAAGCAAAAAAGCGGATTGCATCAGATAAAAA | 189 |
| TCAGAAGCCTCCAACAGGTCAGGATCTGCGAA | 190 |
| CCAAAATATAATGCAGATACATAAACACCAGA | 191 |
| CATTCAACGCGAGAGGCTTTTGCATATTATAG | 192 |
| ACGAGTAGTGACAAGAACCGGATATACCAAGC | 193 |
| AGTAATCTTAAATTGGGCTTGAGAGAATACCA | 194 |
| GCGAAACATGCCACTACGAAGGCATGCGCCGA | 195 |
| ATACGTAAAAGTACAACGGAGATTTCATCAAG | 196 |
| CAATGACACTCCAAAAGGAGCCTTACAACGCC | 197 |
| AAAAAAGGACAACCATCGCCCACGCGGGTAAA | 198 |
| TGTAGCATTCCACAGACAGCCCTCATCTCCAA | 199 |
| GTAAAGCACTAAATCGGAACCCTAGTTGTTCC | 200 |
| AGTTTGGAGCCCTTCACCGCCTGGTTGCGCTC | 201 |
| AGCTGATTACAAGAGTCCACTATTGAGGTGCC | 202 |
| ACTGCCCGCCGAGCTCGAATTCGTTATTACGC | 203 |
| CCCGGGTACTTTCCAGTCGGGAAACGGGCAAC | 204 |
| CAGCTGGCGGACGACGACAGTATCGTAGCCAG | 205 |
| GTTTGAGGGAAAGGGGGATGTGCTAGAGGATC | 206 |
| CTTTCATCCCCAAAAACAGGAAGACCGGAGAG | 207 |
| AGAAAAGCAACATTAAATGTGAGCATCTGCCA | 208 |
| GGTAGCTAGGATAAAAATTTTTAGTTAACATC | 209 |
| CAACGCAATTTTTGAGAGATCTACTGATAATC | 210 |
| CAATAAATACAGTTGATTCCCAATTTAGAGAG | 211 |
| TCCATATACATACAGGCAAGGCAACTTTATTT | 212 |
| TACCTTTAAGGTCTTTACCCTGACAAAGAAGT | 213 |
| CAAAAATCATTGCTCCTTTTGATAAGTTTCAT | 214 |
| TTTGCCAGATCAGTTGAGATTTAGTGGTTTAA | 215 |
| AAAGATTCAGGGGGTAATAGTAAACCATAAAT | 216 |
| TTTCAACTATAGGCTGGCTGACCTTGTATCAT | 217 |
| CCAGGCGCTTAATCATTGTGAATTACAGGTAG | 218 |
| CGCCTGATGGAAGTTTCCATTAAACATAACCG | 219 |
| TTTCATGAAAATTGTGTCGAAATCTGTACAGA | 220 |
| ATATATTCTTTTTTCACGTTGAAAATAGTTAG | 221 |
| AATAATAAGGTCGCTGAGGCTTGCAAAGACTT | 222 |
| CGTAACGATCTAAAGTTTTGTCGTGAATTGCG | 223 |
| ACCCAAATCAAGTTTTTTGGGGTCAAAGAACG | 224 |
| TGGACTCCCTTTTCACCAGTGAGACCTGTCGT | 225 |
| TGGTTTTTAACGTCAAAGGGCGAAGAACCATC | 226 |
| GCCAGCTGCCTGCAGGTCGACTCTGCAAGGCG | 227 |
| CTTGCATGCATTAATGAATCGGCCCGCCAGGG | 228 |
| ATTAAGTTCGCATCGTAACCGTGCGAGTAACA | 229 |
| TAGATGGGGGGTAACGCCAGGGTTGTGCCAAG | 230 |
| ACCCGTCGTCATATGTACCCCGGTAAAGGCTA | 231 |
| CATGTCAAGATTCTCCGTGGGAACCGTTGGTG | 232 |
| TCAGGTCACTTTTGCGGGAGAAGCAGAATTAG | 233 |
| CTGTAATATTGCCTGAGAGTCTGGAAAACTAG | 234 |
| CAAAATTAAAGTACGGTGTCTGGAAGAGGTCA | 235 |
| TGCAACTAAGCAATAAAGCCTCAGTTATGACC | 236 |
| TTTTTGCGCAGAAAACGAGAATGAATGTTTAG | 237 |
| AAACAGTTGATGGCTTAGAGCTTATTTAAATA | 238 |
| ACTGGATAACGGAACAACATTATTACCTTATG | 239 |
| ACGAACTAGCGTCCAATACTGCGGAATGCTTT | 240 |
| CGATTTTAGAGGACAGATGAACGGCGCGACCT | 241 |
| CTTTGAAAAGAACTGGCTCATTATTTAATAAA | 242 |
| GCTCCATGAGAGGCTTTGAGGACTAGGGAGTT | 243 |
| ACGGCTACTTACTTAGCCGGAACGCTGACCAA | 244 |
| AAAGGCCGAAAGGAACAACTAAAGCTTTCCAG | 245 |
| GAGAATAGCTTTTGCGGGATCGTCGGGTAGCA | 246 |
| ACGTTAGTAAATGAATTTTCTGTAAGCGGAGT | 247 |
| TTTTCGATGGCCCACTACGTAAACCGTC | 248 |
| TATCAGGGTTTTCGGTTTGCGTATTGGGAACGCGCG | 249 |
| GGGAGAGGTTTTTGTAAAACGACGGCCATTCCCAGT | 250 |
| CACGACGTTTTTGTAATGGGATAGGTCAAAACGGCG | 251 |
| GATTGACCTTTTGATGAACGGTAATCGTAGCAAACA | 252 |
| AGAGAATCTTTTGGTTGTACCAAAAACAAGCATAAA | 253 |
| GCTAAATCTTTTCTGTAGCTCAACATGTATTGCTGA | 254 |
| ATATAATGTTTTCATTGAATCCCCCTCAAATCGTCA | 255 |
| TAAATATTTTTTGGAAGAAAAATCTACGACCAGTCA | 256 |
| GGACGTTGTTTTTCATAAGGGAACCGAAAGGCGCAG | 257 |
| ACGGTCAATTTTGACAGCATCGGAACGAACCCTCAG | 258 |
| CAGCGAAATTTTAACTTTCAACAGTTTCTGGGATTT | 259 |
| TGCTAAACTTTT | |

TABLE 10
Staple strand sequences modified for the origami with poly-dA.

| | | SEQ ID |
|---|---|---|
| ID | Staple Strand Sequences | NO |
| 100-30A | TATAAGTATAGCCCGGCCGTCGAGAGGGTTGAAAAAAAAAAAAAAAAAAAAAAAAAAAAAAA | 260 |
| 101-30A | ATAAATCCTCATTAAATGATATTCACAAACAAAAAAAAAAAAAAAAAAAAAAAAAAAAAAAA | 261 |
| 102-30A | AATCAGTAGCGACAGATCGATAGCAGCACCGTAAAAAAAAAAAAAAAAAAAAAAAAAAAAAA | 262 |
| 103-30A | TAAAGGTGGCAACATAGTAGAAAATACATACAAAAAAAAAAAAAAAAAAAAAAAAAAAAAAA | 263 |
| 104-30A | GACGGGAGAATTAACTACAGGGAAGCGCATTAAAAAAAAAAAAAAAAAAAAAAAAAAAAAAA | 264 |
| 105-30A | GCTTATCCGGTATTCTAAATCAGATATAGAAGAAAAAAAAAAAAAAAAAAAAAAAAAAAAAA | 265 |
| 106-30A | CGACAAAAGGTAAAGTAGAGAATATAAAGTACAAAAAAAAAAAAAAAAAAAAAAAAAAAAAA | 266 |
| 107-30A | CGCGAGAAAACTTTTTATCGCAAGACAAAGAAAAAAAAAAAAAAAAAAAAAAAAAAAAAAAA | 267 |
| 108-30A | ATTAATTACATTTAACACATCAAGAAAACAAAAAAAAAAAAAAAAAAAAAAAAAAAAAAAAA | 268 |
| 109-30A | TTCATCAATATAATCCTATCAGATGATGGCAAAAAAAAAAAAAAAAAAAAAAAAAAAAAAAA | 269 |
| 110-30A | AATCAATATCTGGTCACAAATATCAAACCCTCAAAAAAAAAAAAAAAAAAAAAAAAAAAAAA | 270 |
| 111-30A | ACCAGTAATAAAAGGGATTCACCAGTCACACGAAAAAAAAAAAAAAAAAAAAAAAAAAAAAA | 271 |
| 205-30A | CGATGGCCCACTACGTAAACCGTCTATCAGGGAAAAAAAAAAAAAAAAAAAAAAAAAAAAAA | 272 |
| 206-30A | CGGTTTGCGTATTGGGAACGCGCGGGGAGAGGAAAAAAAAAAAAAAAAAAAAAAAAAAAAAA | 273 |
| 207-30A | TGTAAAACGACGGCCATTCCCAGTCACGACGTAAAAAAAAAAAAAAAAAAAAAAAAAAAAAA | 274 |
| 208-30A | GTAATGGGATAGGTCAAAACGGCGGATTGACCAAAAAAAAAAAAAAAAAAAAAAAAAAAAAA | 275 |
| 209-30A | GATGAACGGTAATCGTAGCAAACAAGAGAATCAAAAAAAAAAAAAAAAAAAAAAAAAAAAAA | 276 |
| 210-30A | GGTTGTACCAAAAACAAGCATAAAGCTAAATCAAAAAAAAAAAAAAAAAAAAAAAAAAAAAA | 277 |
| 211-30A | CTGTAGCTCAACATGTATTGCTGAATATAATGAAAAAAAAAAAAAAAAAAAAAAAAAAAAAA | 278 |
| 212-30A | CATTGAATCCCCCTCAAATCGTCATAAATATTAAAAAAAAAAAAAAAAAAAAAAAAAAAAAA | 279 |
| 213-30A | GGAAGAAAAATCTACGACCAGTCAGGACGTTGAAAAAAAAAAAAAAAAAAAAAAAAAAAAAA | 280 |
| 214-30A | TCATAAGGGAACCGAAAGGCGCAGACGGTCAAAAAAAAAAAAAAAAAAAAAAAAAAAAAAAA | 281 |
| 215-30A | GACAGCATCGGAACGAACCCTCAGCAGCGAAAAAAAAAAAAAAAAAAAAAAAAAAAAAAAAA | 282 |
| 216-30A | AACTTTCAACAGTTTCTGGGATTTTGCTAAACAAAAAAAAAAAAAAAAAAAAAAAAAAAAAA | 283 |

To determine the concentrations of the DNA origami templates, their absorbance at 260 nm was measured by NanoDrop Lite Spectrophotometer (ThermoFisher Scientific). The extinction coefficient of each DNA origami template design can be approximated by equation (9).^{[44]}

$$ε = 6 \; 700 ds + 10 \; 000 ss \qquad ( 9 )$$

where ds is the number of double stranded bases and ss is the number of single-stranded bases. Using Lambert-Beer's law (A260 nm=εabc, b=1 cm), the concentrations of DNA origami templates can be calculated.

TABLE 11
Sequences used in the silencing experiments.
Bases highlighted are FANA ,bases
underlined are phosphorotioated.^{[35]}

| | | SEQ ID |
|---|---|---|
| Name | Sequence (5′→3′) | NO |
| ASO | AUAUCCTTGTCGTAUCCC | 284 |
| Scramble | AAUAATTGAATTCTUCAA | 285 |
| ASO-Integrated | AAAAAAAAAAAAAAAAAA | 286 |
| | AAAAAAAAAAAAAUAUCC | |
| | TTGTCGTAUCCC | |
| Scramble- | AAAAAAAAAAAAAAAAAA | 287 |
| Integrated | AAAAAAAAAAAAAAUAAT | |
| | TGAATTCTUCAA | |
| ASO-Complement | AAAAAAAAAAAAAAAAAA | 288 |
| | AAAAAAAAAAAATTACGG | |
| | GATACGACAA | |
| Scramble- | AAAAAAAAAAAAAAAAAA | 289 |
| Complement | AAAAAAAAAAAATTACTT | |
| | GAAGAATTCAATT | |
| dA₃₀C₈ | AAAAAAAAAAAAAAAAAA | 290 |
| | AAAAAAAAAAAACCCCCC | |
| | CC | |

### Example 5-Double-Stranded DNA Protection

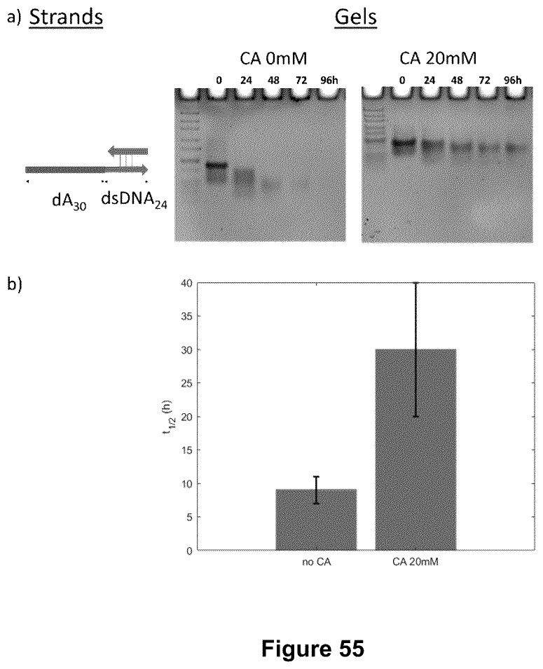

With the investigation of the patterns of protection of single-stranded overhangs from nucleases in the presence of an adjacent polyA/CA tail, we were interested in testing if the same mechanism applies to double-stranded overhangs. To this effect, we have added a complement to the overhang of the strand denoted dA₃₀-ssDNA₂₄ to generate the construct we have denoted dA₃₀ (FIG. 55).

TABLE 12
Half-lives of DNA 1:1 and dA₃₀ with different enzymes.
The p-value statistical validity of having different
nuclease resistance compared to having no CA-R present.

| | CA 0 mM | | CA 20 mM | | |
|---|---|---|---|---|---|
| Sequence | t_{1/2} | (h) | t_{1/2} | (h) | p-value |
| dA30 (Exo I) | <<1 | | 63 | ±15 | — |
| DNA 1:1 (Exo I) | 0.54 | ±0.21 | 1.2 | ±0.4 | 0.97 |
| dA30 (DNAse I) | 0.46 | ±0.11 | >120 | | — |

The results shown in FIG. 8 and Table 12 demonstrate the increased stability in the presence of CA from t_{1/2}=9±2 h to t_{1/2}=30±10 h. While the stability increase is significant, it is lower than that of the same strand with only single-stranded overhang (t_{1/2}=120±20 h), indicating that protecting a double-stranded region is more challenging than a single-stranded one for this system. Looking more closely to the degradation pattern (FIG. 55, a, right panel), it appears the dA₃₀-dsDNA₂₄ degrades into its single-stranded form dA₃₀-ssDNA₂₄, or a strand with close nucleotide length. Once it has reached this length, it remains present until the end of the collection window, as the original dA₃₀-ssDNA₂₄.

Further study with different lengths of complementary strands and geometry of polyA tail attachment (such as polyA tails on both strands of the dsDNA) may lead to improvements in stabilization, in addition of free dA₃₀ supplementation as demonstrated with single-stranded overhangs.

### Example 6-Cyanuric Acid (CA-R) Sidechains

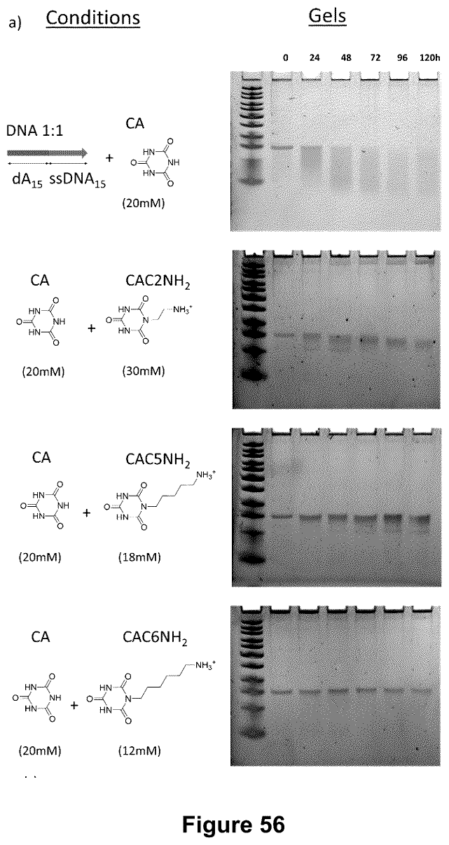

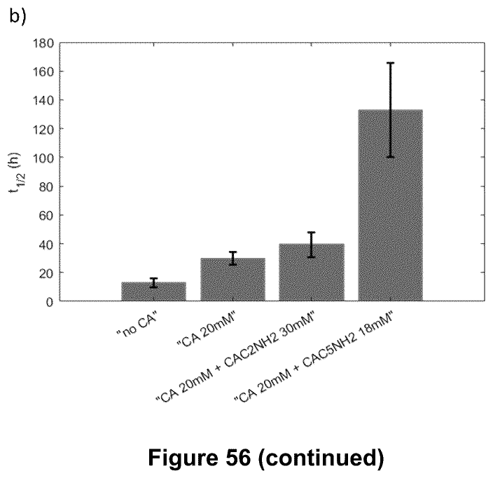

From earlier studies, the polyA/CA assembly could not only tolerate sidechains on the non-binding face of the cyanuric acid, but that these sidechains had significant structural and thermodynamic effects on the resulting assemblies. For example, amino sidechains neutralized the negative phosphate backbone, which increased stability and inter-fiber interactions. The effect of these morphological changes on the resulting nuclease resistance of the sequences was tested by the addition of CAC2NH₂, CAC5NH₂ or CAC6NH₂ (FIG. 56). The sequence used for evaluating the nuclease resistance is the polyA:DNA 1:1 ratiometric sequence, since it only gained limited protection with CA, allowing any changes in that protection to be reliably tracked.

TABLE 13
Half-lives of DNA 1:1 with various CA-R compounds. The p-value statistical validity
of having different nuclease resistance compared to having no CA-R present.

| | | | | Ratio | | |
|---|---|---|---|---|---|---|
| Sequence | Conditions | t_{1/2} (h) | | CA-R/CA0 | | p-value |
| DNA 1:1 | | 13 | ±3 | | | |
| DNA 1:1 | CA 20 mM | 30 | ±4 | 2.3 | ±0.6 | 0.98 |
| DNA 1:1 | CA 20 mM + CAC2NH2 | 39 | ±9 | 3.1 | ±0.4 | 0.98 |
| | 30 mM | | | | | |
| DNA 1:1 | CA 20 mM + CAC5NH2 | 130 | ±30 | 10.2 | ±0.3 | 0.99 |
| | 18 mM | | | | | |
| DNA 1:1 | CA 20 mM + CAC6NH2 | >130 | | >10 | | |
| | 12 mM | | | | | |

The results of these experiments and the half-lives in FIG. 56 and Table 13. All the derivatives in this series increase the half-life of the sequence. This increase in half-life follows the increasing length of the alkyl linker, with the addition of CAC6NH₂ inhibiting observable degradation in the time range observed. In a previous study of CA sidechains, it was observed that positively charged ammonium groups linked through longer alkyl linkers mediate fiber-fiber interactions by binding phosphates of other fibers. The short CAC2NH₂ prefers to bind phosphates intramolecularly, while bundled fibers are observed with CAC5NH₂, and bundling was greatest with the CAC6NH₂ derivative. The trends in nuclease resistance mirror the bundling propensity of the cyanuric acid derivative, which supports the hypothesis that the protection of adjacent sequences is through their sequestration in local, bundled polyA/CA-R networks.

Tuning the nuclease protection through the small-molecule's chemistry demonstrates the advantages of the polyA/CA-R hybrid structure. Modifications of the small molecule can be achieved at low cost, independently from the production of the nucleic acid strands. The nature of the nuclease protection observed allows for tuning with the wide supramolecular toolkit, such as inter-fiber interactions, providing a breadth of methodologies to tackle challenges in therapeutic nucleic acids. From these initial hits, further development in CA-R could target desired nuclease resistance properties, as well as other valuable pharmaceutical aspects, such as the lowering of the necessary concentrations and increase in pH.

Cellular viability. To be applicable in biological context, the toxicity of the CA and the combined CA-R mixes were verified. Epithelial cells were cultivated in media supplemented with various CA-R mixes and acidified at pH 6. Both the cellular viability and morphology was studied over a 5 day period.

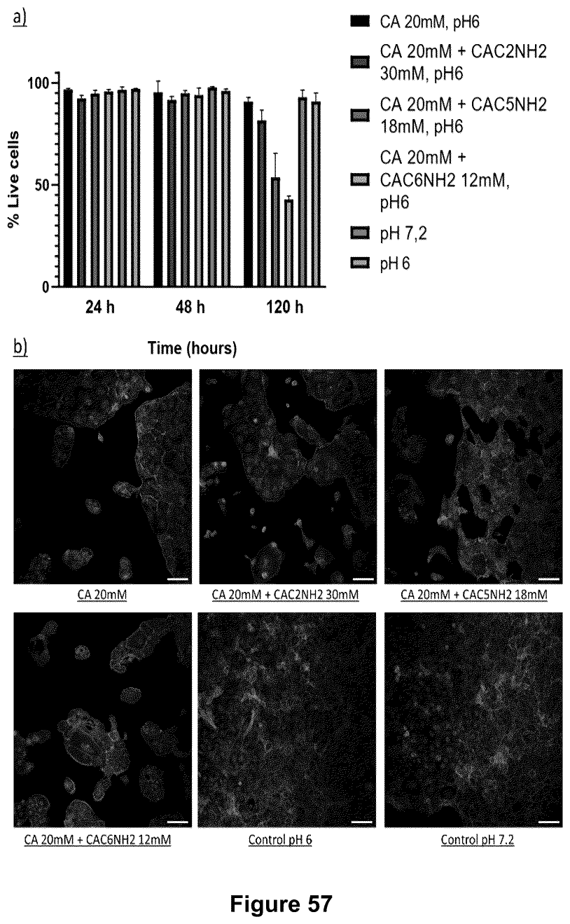

The viability for all CA-R mixes up to 48 h is above 90%, overlapping with the viability from the controls (FIG. 57). For CA 20 mM, this remains the case after 5 days, with a viability of 91±2%. When CA-R are added, the viability decreases with the length of the alkylamino sidechain length, from 82±5% with CAC2NH₂ to 43±2% with CAC6NH₂. This trend could reflect the ability of the longer, more hydrophobic sidechains to enter cells, but the precise mechanism needs elucidation. The morphology shows more pockets of isolated cells with CA-R mixes, but they are still able to grow and form cell-cell contacts.

The use of CA low toxicity in vitro was observed. For the CA-R, the improvements in DNA lifetimes result in higher toxicity. With further investigations, it is possible the bundling and protective ability could be disentangled from the damage to cells, for example by replacing the C₆ chain with an ethylene glycol chain.

Although the embodiments have been described in detail, it should be understood that various changes, substitutions and alterations can be made herein. Moreover, the scope of the present application is not intended to be limited to the particular embodiments or examples described in the specification. As can be understood, the examples described above and illustrated are intended to be exemplary only.

For example, the present invention contemplates that any of the features shown in any of the embodiments described herein, may be incorporated with any of the features shown in any of the other embodiments described herein, and still fall within the scope of the present invention.

### Example 1 References

Kulkarni, J. A.; Witzigmann, D.; Thomson, S. B.; Chen, S.; Leavitt, B. R.; Cullis, P. R.; van der Meel, R., The current landscape of nucleic acid therapeutics. Nature Nanotechnology 2021, 16 (6), 630-643.
Dolgin, E., The tangled history of mRNA vaccines. Nature 2021, 597 (7876), 318-324.
Lacroix, A.; Fakih, H. H.; Sleiman, H. F., Detailed cellular assessment of albumin-bound oligonucleotides: Increased stability and lower non-specific cell uptake. J Control Release 2020, 324, 34-46.
Biolabs, N. E. Properties of Exonucleases and Non-specific Endonucleases. https://www.neb.com/tools-and-resources/selection-charts/properties-of-exonucleases-and-nonspecific-endonucleases.
Bujold, K. E.; Lacroix, A.; Sleiman, H. F., DNA Nanostructures at the Interface with Biology. Chem 2018, 4 (3), 495-521.
Rizzuto, F. J.; Platnich, C. M.; Luo, X.; Shen, Y.; Dore, M. D.; Lachance-Brais, C.; Guarne, A.; Cosa, G.; Sleiman, H. F., A dissipative pathway for the structural evolution of DNA fibres. Nat Chem 2021, 13 (9), 843-849.
Lachance-Brais, C.; Hennecker, C. D.; Alenaizan, A.; Luo, X.; Toader, V.; Taing, M.; Sherrill, C. D.; Mittermaier, A. K.; Sleiman, H. F., Tuning DNA Supramolecular Polymers by the Addition of Small, Functionalized Nucleobase Mimics. J. Am. Chem. Soc. 2021, 143 (47), 19824-19833.
Zagorovsky, K.; Chou, L. Y.; Chan, W. C., Controlling DNA-nanoparticle serum interactions. Proc Natl Acad Sci USA 2016, 113 (48), 13600-13605.

### Example 2 References

Lachance-Brais, C.; Hennecker, C. D.; Alenaizan, A.; Luo, X.; Toader, V.; Taing, M.; Sherrill, C. D.; Mittermaier, A. K.; Sleiman, H. F. Tuning DNA Supramolecular Polymers by the Addition of Small, Functionalized Nucleobase Mimics. J. Am. Chem. Soc. 2021, 143 (47), 19824-19833. https://doi.org/10.1021/jacs.1c08972.
Reprogramming the assembly of unmodified DNA with a small molecule | Nature Chemistry. https://www.nature.com/articles/nchem.2451 (accessed 2022 Dec. 20).

### Example 3 References

O. Jeon, K. Lee, E. Alsberg, Small 2018, 14, n/a; b) Y. Zhang, C. Fu, Y. Li, K. Wang, X. Wang, Y. Wei, L. Tao, Polymer Chemistry 2017, 8, 537.
X. Huang, R. Zheng, F. Ding, J. Yang, M. Xie, X. Liu, J. Li, J. Feng, X. Zhu, C. Zhang, ACS Materials Letters 2020, 2, 1509; b) J. Zhang, Y. Guo, G. Pan, P. Wang, Y. Li, X. Zhu, C. Zhang, ACS Appl. Mater. Interfaces 2020, 12, 21441; c) S. Han, Y. Park, H. Kim, H. Nam, O. Ko, J. B. Lee, ACS Appl. Mater. Interfaces 2020, 12, 55554.
Y. Shao, Z.-Y. Sun, Y. Wang, B.-D. Zhang, D. Liu, Y.-M. Li, ACS Appl. Mater. Interfaces 2018, 10, 9310.
S. Kim, J. Fan, C.-S. Lee, C. Chen, M. Lee, ACS Appl. Bio Mater. 2021, 4, 5189.
F. Huang, M. Chen, Z. Zhou, R. Duan, F. Xia, I. Willner, Nat. Commun. 2021, 12, 2364; b) T. Yuan, Y. Shao, X. Zhou, Q. Liu, Z. Zhu, B. Zhou, Y. Dong, N. Stephanopoulos, S. Gui, H. Yan, D. Liu, Adv. Mater. (Weinheim, Ger.)₂₀₂₁, Ahead of Print; c) Y. Wang, Y. Shao, X. Ma, B. Zhou, A. Faulkner-Jones, W. Shu, D. Liu, ACS Appl. Mater. Interfaces 2017, 9, 12311.
Y. Gu, M. E. Distler, H. F. Cheng, C. Huang, C. A. Mirkin, J Am Chem Soc 2021.
M. Oishi, K. Nakatani, Small 2019, Ahead of Print; b) L. Yue, S. Wang, V. Wulf, I. Willner, Nat. Commun. 2019, 10, 1.
L. Yue, S. Wang, Z. Zhou, I. Willner, J. Am. Chem. Soc. 2020, 142, 21577.
R. Merindol, G. Delechiave, L. Heinen, L. H. Catalani, A. Walther, Nature Communications 2019, 10, 528.
J. Shi, C. Zhu, Q. Li, Y. Li, L. Chen, B. Yang, J.-F. Xu, Y. Dong, C. Mao, D. Liu, Macromolecular Rapid Communications 2021, 42, 2100182.
C. F. Guimarães, L. Gasperini, A. P. Marques, R. L. Reis, Nature Reviews Materials 2020, 5, 351.
M. Madsen, K. V. Gothelf, Chemical Reviews 2019, 119, 6384.
N. Avakyan, A. A. Greschner, F. Aldaye, C. J. Serpell, V. Toader, A. Petitjean, H. F. Sleiman, Nat Chem 2016, 8, 368.
C. Lachance-Brais, C. D. Hennecker, A. Alenaizan, X. Luo, V. Toader, M. Taing, C. D. Sherrill, A. K. Mittermaier, H. F. Sleiman, J Am Chem Soc 2021, 143, 19824; b) A. Alenaizan, K. Fauche, R. Krishnamurthy, C. D. Sherrill, Chemistry 2020.
F. J. Rizzuto, C. M. Platnich, X. Luo, Y. Shen, M. D. Dore, C. Lachance-Brais, A. Guarne, G. Cosa, H. F. Sleiman, Nat Chem 2021, 13, 843.
J. Shi, C. Zhu, Q. Li, Y. Li, L. Chen, B. Yang, J.-F. Xu, Y. Dong, C. Mao, D. Liu, Macromol. Rapid Commun. 2021, 42, 2100182.
D. Calvet, J. Y. Wong, S. Giasson, Macromolecules 2004, 37, 7762.
W. Helen, P. de Leonardis, R. V. Ulijn, J. Gough, N. Tirelli, Soft Matter 2011, 7, 1732; b) E. T. Pashuck, B. J. R. Duchet, C. S. Hansel, S. A. Maynard, L. W. Chow, M. M. Stevens, ACS Nano 2016, 10, 11096; c) E. Takai, G. Ohashi, R. Ueki, Y. Yamada, J. I. Fujita, K. Shiraki, American Journal of Biochemistry and Biotechnology 2014, 10, 31.
Z. Yang, B. Xu, Chemical Communications 2004, 2424; b) Z. Yang, H. Gu, D. Fu, P. Gao, J. K. Lam, B. Xu, Advanced Materials 2004, 16, 1440.
Q. Hu, K. Dong, J. Ming, W. Yang, H. Wang, X. Xiao, T. Huang, Mater. Today Chem. 2022, 23, 100680; b) Y. Jin, Y. Li, S. Song, Y. Ding, Y. Dong, Y. Lu, D. Liu, C. Zhang, Adv. Mater. Interfaces 2022, 9, 2101321; c) M. He, N. Nandu, T. B. Uyar, M. Royzen, M. V. Yigit, Chem. Commun. (Cambridge, U. K.) 2020, 56, 7313; d) G. Creusen, C. O. Akintayo, K. Schumann, A. Walther, J Am Chem Soc 2020; e) X. Zhou, C. Li, Y. Shao, C. Chen, Z. Yang, D. Liu, Chemical Communications 2016, 52, 10668; f) E. Cheng, Y. Xing, P. Chen, Y. Yang, Y. Sun, D. Zhou, L. Xu, Q. Fan, D. Liu, Angew. Chem., Int. Ed. 2009, 48, 7660.
S. Basu, A. Chakraborty, A.-R. I. Alkiswani, Y. Shamiya, A. Paul, Mater. Adv. 2022, 3, 946; b) Q. Chen, S. Wang, T. Huang, F. Xiao, Z. Wu, R. Yu, Anal. Chem. (Washington, DC, U. S.) 2022, 94, 5530; c) C. Li, X. Zhou, Y. Shao, P. Chen, Y. Xing, Z. Yang, Z. Li, D. Liu, Materials Chemistry Frontiers 2017, 1, 654; d) C. Li, M. J. Rowland, Y. Shao, T. Cao, C. Chen, H. Jia, X. Zhou, Z. Yang, O. A. Scherman, D. Liu, Advanced Materials 2015, 27, 3298; e) C. Li, P. Chen, Y. Shao, X. Zhou, Y. Wu, Z. Yang, Z. Li, T. Weil, D. Liu, Small 2015, 11, 1138; f) S. Y. Ahn, J. Kim, S. Vellampatti, S. Oh, Y. T. Lim, S. H. Park, D. Luo, J. Chung, S. H. Um, Advanced Materials 2022, 2110424.
Y. Shao, H. Jia, T. Cao, D. Liu, Acc. Chem. Res. 2017, 50, 659.
L. L. Wang, J. J. Chung, E. C. Li, S. Uman, P. Atluri, J. A. Burdick, Journal of Controlled Release 2018, 285, 152.
C. B. Highley, C. B. Rodell, J. A. Burdick, Adv. Mater. (Weinheim, Ger.) 2015, 27, 5075.
X.-a. Zhang, S.-I. Jiang, J. Li, Y.-f. Lv, Gaofenzi Tongbao 2016, 1.
R. E. Newnham, G. R. Ruschau, J. Am. Ceram. Soc. 1991, 74, 463.
F. C. Simmel, B. Yurke, H. R. Singh, Chemical Reviews 2019, 119, 6326.
E. E. Simanek, X. Li, I. S. Choi, G. M. Whitesides, 1996.
Y. Cai, E. Lopez-Ruiz, J. Wengel, L. B. Creemers, K. A. Howard, J. Controlled Release 2017, 253, 153.
D. Wang, L. Yu, C.-M. Huang, G. Arya, S. Chang, Y. Ke, J Am Chem Soc 2021, 143, 2256; b) S. Gentile, E. Del Grosso, L. J. Prins, F. Ricci, Angewandte Chemie International Edition, n/a.
L. Qian, E. Winfree, J. Bruck, Nature (London, U. K.) 2011, 475, 368.
S. Wang, Z. Zhou, N. Ma, S. Yang, K. Li, C. Teng, Y. Ke, Y. Tian, Sensors 2020, 20, 6899.
H. Pei, L. Liang, G. Yao, J. Li, Q. Huang, C. Fan, Angew. Chem., Int. Ed. 2012, 51, 9020; b) D. Wang, Y. Fu, J. Yan, B. Zhao, B. Dai, J. Chao, H. Liu, D. He, Y. Zhang, C. Fan, S. Song, Anal. Chem. (Washington, DC, U. S.) 2014, 86, 1932; c) A. R. Chandrasekaran, O. Levchenko, D. S. Patel, M. Maclsaac, K. Halvorsen, Nucleic Acids Res. 2017, 45, 11459.
S. Fan, B. Ji, Y. Liu, K. Zou, Z. Tian, B. Dai, D. Cui, P. Zhang, Y. Ke, J. Song, Angewandte Chemie International Edition 2022, 61, e202116324; b) V. Linko, M. Eerikäinen, M. A. Kostiainen, Chemical Communications 2015, 51, 5351.
S. Jiang, Z. Ge, S. Mou, H. Yan, C. Fan, Chem 2021, 7, 1156.
H. H. Fakih, A. Katolik, E. Malek-Adamian, J. J. Fakhoury, S. Kaviani, M. J. Damha, H. F. Sleiman, Chemical Science 2021, 12, 2993.
Y. Zhu, L. Zhu, X. Wang, H. Jin, Cell Death & Disease 2022, 13, 644.
U.S. National Library of Medecine, clinicaltrials.gov 2022.
A. Kalota, L. Karabon, C. R. Swider, E. Viazovkina, M. Elzagheid, M. J. Damha, A. M. Gewirtz, Nucleic Acids Research 2006, 34, 451; b) M. Takahashi, H. Li, J. Zhou, P. Chomchan, V. Aishwarya, M. J. Damha, J. J. Rossi, Molecular Therapy-Nucleic Acids 2019, 17, 615.
N. Carballo-Pedrares, I. Fuentes-Boquete, S. Díaz-Prado, A. Rey-Rico, Pharmaceutics 2020, 12, 752.
P. W. K. Rothemund, Nature 2006, 440, 297.
J. F. Rahbani, A. A. Hariri, G. Cosa, H. F. Sleiman, ACS Nano 2015, 9, 11898.
C.-N. Lok, E. Viazovkina, K.-L. Min, E. Nagy, C. J. Wilds, M. J. Damha, M. A. Parniak, Biochemistry 2002, 41, 3457

1. A nucleic acid hydrogel for delivery of a therapeutic oligonucleotide, the nucleic acid hydrogel comprising a biocompatible polymer grafted to a nucleic acid strand having a polyadenine segment or grafted to a thymine-mimicking compound, the polyadenine segment forming a supramolecular polymer with the thymine-mimicking compound; wherein the nucleic acid strand is bonded to the therapeutic oligonucleotide by covalent bonding or complementary base pairing; wherein the thymine-mimicking compound is cyanuric acid (CA), xanthine, barbituric acid, uric acid, or a derivative thereof having a positively charged side chain; and wherein the supramolecular polymer is configured to dissociate to release the therapeutic oligonucleotide.

2. The nucleic acid hydrogel of claim 1, wherein the polyadenine segment comprises DNA or RNA adenine nucleotides or modified nucleotides thereof.

3. The nucleic acid hydrogel of claim 2, wherein the polyadenine segment comprises between 10 to 50_at least 10-adenine nucleotides, more preferably 15 to 30 adenine nucleotides.

4. The nucleic acid hydrogel of claim 3, wherein the polyadenine segment further comprises 4-8 cytosine nucleotides.

5. The nucleic acid hydrogel of claim 1, wherein the nucleic acid strand is covalently bonded to the 5′ or 3′ end of the therapeutic oligonucleotide.

6. The nucleic acid hydrogel of claim 1, wherein the nucleic acid strand comprises a first overhang segment having a nucleic acid sequence complementary to the therapeutic oligonucleotide for base pairing with the therapeutic oligonucleotide.

7. The nucleic acid hydrogel of claim 1, wherein the nucleic acid strand comprises one or more flanking segments adjacent to or in the middle of the polyadenine segment, the one or more flanking segments comprising an internal hairpin or a double-stranded DNA region.

8. (canceled)

9. The nucleic acid hydrogel of claim 5, wherein the ratio of adenine nucleotides to non-adenine nucleotides in the nucleic acid strand is between 1:1 and 2:3.

10. The nucleic acid hydrogel of claim 6, wherein the nucleic acid strand comprises a second overhang segment for base pairing with a complementary crosslinking overhang segment of the biocompatible polymer to form the supramolecular polymer.

11. The nucleic acid hydrogel of claim 10, wherein the second overhang segment base pairs with the complementary crosslinking overhang segment to form a double-stranded DNA segment, an i-motif, or a G-quadruplex.

12. (canceled)

13. (canceled)

14. (canceled)

15. (canceled)

16. The nucleic acid hydrogel of claim 1, wherein the thymine-mimicking compound is a cyanuric acid derivative, the cyanuric acid derivative having the side chain conjugated to one of the nitrogens, the side chain being-(CH₂)ₙ—NH₂, where n is 2 to 6.

17. The nucleic acid hydrogel of claim 1, comprising 12-90 mM of CA or a derivative thereof.

18. The nucleic acid hydrogel of claim 1, wherein the supramolecular polymer is configured to dissociate when stimulated by pH changes or by introduction of a competing compound to the thymine-mimicking compound.

19. The nucleic acid hydrogel of claim 1, wherein the therapeutic oligonucleotide is:
i) an antisense oligonucleotide;
ii) a therapeutic aptamer; or
iii) a mRNA, small interfering RNA (siRNA), microRNA (miRNA), or short hairpin RNA (shRNA).

20. (canceled)

21. (canceled)

22. A method of gene silencing, the method comprising administering a patient with the nucleic acid hydrogel of claim 19.

23. A method of forming the nucleic acid hydrogel of claim 1 to protect the protecting a therapeutic oligonucleotide from nuclease degradation for drug delivery, the method comprising:
i) bonding the therapeutic oligonucleotide to the nucleic acid strand; and
ii) contacting the bonded therapeutic oligonucleotide nucleic acid strand with the thymine-mimicking compound, under suitable conditions for the polyadenine segment to form the nucleic acid hydrogel, wherein the nucleic acid hydrogel protects the therapeutic oligonucleotide from nucleases.

24. (canceled)

25. (canceled)

26. (canceled)

27. (canceled)

28. (canceled)

29. (canceled)

30. The method of claim 23, wherein forming the nucleic acid hydrogel comprises contacting the bonded therapeutic oligonucleotide nucleic acid strand with another biocompatible polymer having a polyadenine segment.

31. The method of claim 10, wherein the biocompatible polymer comprises a third overhang segment for base pairing with a complementary crosslinking overhang segment of a further nucleic acid hydrogel.

32. The method of claim 31, comprising further forming a double network polymer mixture with another biocompatible polymer.

33. The method of claim 23, further comprising stimulating the nucleic acid hydrogel to dissociate thereby releasing the bonded therapeutic oligonucleotide by
increasing the pH preferably to physiological pH; or
contacting the nucleic acid hydrogel with a further polythymine strand, melamine or derivatives thereof.

34. (canceled)

35. (canceled)

36. (canceled)

37. (canceled)

38. (canceled)

39. A kit comprising the biocompatible polymer, the nucleic acid strand, and the thymine-mimicking compound as defined in claim 1.

40. A kit of claim 39, wherein the biocompatible polymer is grafted to the thymine-mimicking.

41. The method of claim 23 forming a nucleic acid hydrogel, the method comprising:
further providing a free polyadenine nucleic acid strand;
wherein contacting the bonded therapeutic oligonucleotide nucleic acid strand with the thymine-mimicking compound comprises contacting the bonded therapeutic oligonucleotide nucleic acid strand and the free polyadenine nucleic acid strand with the thymine-mimicking compound, under suitable conditions for the polyadenine segment and the free polyadenine nucleic acid strand to form the nucleic acid hydrogel.

42. The method of claim 41, wherein the free polyadenine nucleic acid strand comprises DNA or RNA adenine nucleotides or modified nucleotides thereof.

43. The method of claim 42, wherein the free polyadenine nucleic acid strand comprises at least 10 adenine nucleotides, preferably at least 15 adenine nucleotides.

44. The method of claim 43, wherein the free polyadenine nucleic acid strand comprises between to 300 adenine nucleotides, preferably 15 to 50 adenine nucleotides, more preferably 15 to 30 adenine nucleotides.

45. The method of claim 41, wherein the ratio of adenine nucleotides to non-adenine nucleotides in the conjugated nucleic acid molecule is between 1:1 to 1:3.

46. The nucleic acid hydrogel of claim 17, comprising 20-60 mM of CA or 12-90 mM of a CA derivative, or a combination thereof.

47. The nucleic acid hydrogel of claim 1, comprising 420 μM-2 mM of the polyadenine segment.

48. The nucleic acid hydrogel of claim 1, further comprising free polyadenine strands.
